(19) United States
(12) Patent Application Publication
(10) Pub. No.: US 2023/0118577 A1
(43) Pub. Date: Apr. 20, 2023

(54) NEUROACTIVE STEROIDS AND PHARMACEUTICAL COMPOSITION CONTAINING THE SAME

(71) Applicant: Brii Biosciences, Inc., Durham, NC (US)

(72) Inventors: Lianhong XU, Durham, NC (US); Guiling ZHAO, Durham, NC (US)

(21) Appl. No.: 17/792,319

(22) PCT Filed: Jan. 12, 2021
(86) PCT No.: PCT/US21/13112

§ 371 Date: Jul. 12, 2022

Related U.S. Application Data
(60) Provisional application No. 62/959,977, filed on Jan. 12, 2020.

(51) Int. Cl. C07J 17/00; C07J 43/00

(52) U.S. Cl. CPC C07J 17/00; C07J 43/003

(57) ABSTRACT

The invention of this disclosure is directed to a neuroactive steroid (NAS) of a novel structure. This invention is also directed to a pharmaceutical composition comprising the neuroactive steroid (NAS) and salts thereof. The pharmaceutical composition can be used for preventing and/or treating CNS conditions or diseases related to GABA-modulation, such as depression, bipolar disorder, dementia, Huntington's disease, Parkinson's disease, etc. This invention is further directed to a method for treating a CNS disorder in a subject in need thereof.

### CROSS REFERENCE TO RELATED APPLICATIONS

This application claims the benefit of and priority to U.S. Provisional Application Ser. No. 62/959,977, filed Jan. 12, 2020, which is herein incorporated by reference in its entirety.

### FIELD

This disclosure is directed to neuroactive steroids (NASs) and pharmaceutical compositions comprising the same. This disclosure is further directed to a method for treating a central nervous system (CNS) disorder using a neuroactive steroid.

### BACKGROUND

Neuroactive steroids (NASs) (including neurosteroids (NS)) are modulators of γ-aminobutyric acid (GABA) receptor complex (GRC) in the central nervous system (CNS). The prime target of NASs is the inhibitory GABA Type A receptors (GABA_{A}Rs) that contribute to the modulation of neuronal excitability and rapid mood changes. NASs can be produced de novo in the brain from cholesterol or derived from the local metabolism of peripherally derived steroid precursors. The endogenous neurosteroids can include 5α-pregnane-3α-ol-20-one (allopregnanolone, also known as brexanolone) and 5α-pregnane-3α,21-diol-20-one (THDOC) (Majewska M D, et al., Science. 232:1004-7, 1986 [PubMed: 2422758]). Synthetic neuroactive steroids, such as alphaxalone, can also selectively potentiate responses to GABA (Harrison N L, et al., J Physiol (Lond). 346:42, 1984). Many CNS disorders including a variety of behavioral states such as anxiety levels, panic, stress response, seizures, sleep, vigilance and memory, etc., can be related to GABA_{A}Rs function and can be influenced by NASs and their synthetic derivatives (Zorumski, C F., et al., Neurosci. Biobehavioral Rev. 37:109-122, 2013. DOI: 10.1016/j.neubiorev.2012.10.005).

Given its critical role in the function of neuronal circuits, GABA_{A}Rs are the target for numerous clinically relevant drugs. Brexanolone (also known as allopregnanolone) and ganaxolone are known positive allosteric modulators of the GABA_{A}Rs causing a global inhibition of central nervous system (CNS). A brexanolone solution formulation product for intravenous injection, ZULRESSO®, developed by and sold under registered trademark of Sage Therapeutics (Cambridge, Mass., USA), was recently approved by US Food and Drug Administration (FDA, March 2019) for the treatment of postpartum depression (PPD), a serious and potentially life-threatening condition, for which no current pharmacotherapies are specifically indicated. However, ZULRESSO is inconvenient to use and requires administration to a patient by continuous intravenous (IV) infusion that lasts for a total of about 60 hours (2.5 days). A new oral drug, positive allosteric modulator of GABA_{A}Rs, known as SAGE-217, has shown mixed results in reduction of depressive symptoms in clinical trials after administration of the drug for 14 days. In addition, SAGE-217 exhibited more adverse events than the placebo (Gunduz-Bruce, H., et al., N. Engl. J. Med. 381(10):903-911, September 2019; U.S. Pat. No. 9,512,165 and PCT Publication No.: WO2014/169833). Many new molecules have also been proposed, such as those disclosed in U.S. Pat. No. 9,777,037 and US Patent Publication No.: 20180340005A1. However, their effectiveness in treating human CNS disorders is not clear.

Therefore, better compounds for modulating the functions of GABA_{A}Rs and for the treatment of CNS disorders are needed.

### SUMMARY

The present disclosure is directed to a neuroactive steroid (NAS) of formula (1):

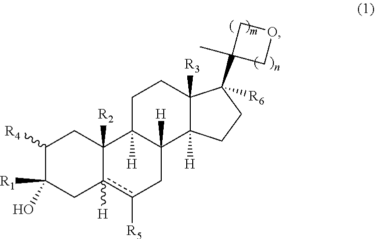

one or more isomers thereof, a deuterium-labeled variant thereof, or a combination thereof, wherein: R₁ is independently H, D, substituted or unsubstituted C1-C10 alkyl, C1-C5 deuterated alkyl, substituted or unsubstituted C2-C10 alkenyl, substituted or unsubstituted C2-C10 alkynyl, substituted or unsubstituted C3-C10 cycloalkyl, substituted or unsubstituted C3-C10 cycloalkenyl, substituted or unsubstituted C3-C10 heterocycloalkyl, substituted or unsubstituted C3-C10 heterocycloalkenyl, substituted or unsubstituted aryl, or substituted or unsubstituted heteroaryl; R₂, R₄ and R₅ each is independently H, halogen, —CN, substituted or unsubstituted C1-C10 alkyl, substituted or unsubstituted C2-C10 alkenyl, substituted or unsubstituted C2-C10 alkynyl, substituted or unsubstituted C3-C10 cycloalkyl, substituted or unsubstituted C3-C10 cycloalkenyl, substituted or unsubstituted C3-C10 heterocycloalkyl, substituted or unsubstituted C3-C10 heterocycloalkenyl, substituted or unsubstituted aryl, or substituted or unsubstituted heteroaryl; R₃ is H, D, halogen, —CN, substituted or unsubstituted C1-C10 alkyl, —CD₃, substituted or unsubstituted C2-C10 alkenyl, substituted or unsubstituted C2-C10 alkynyl, substituted or unsubstituted C3-C10 cycloalkyl, substituted or unsubstituted C3-C10 cycloalkenyl, substituted or unsubstituted C3-C10 heterocycloalkyl, substituted or unsubstituted C3-C10 heterocycloalkenyl, substituted or unsubstituted aryl, or substituted or unsubstituted heteroaryl; R₆ is H or D; and m and n each is independently 0, 1, 2 or 3, with the proviso that at least one of m and n is not zero.

The present invention is also directed to a pharmaceutical composition comprising a neuroactive steroid (NAS) of formula (1), one or more isomers thereof, a deuterium labeled variant thereof, a pharmaceutically acceptable salt thereof, or a combination thereof; and a pharmaceutically acceptable excipient; wherein: R₁ is independently H, D, substituted or unsubstituted C1-C10 alkyl, C1-C5 deuterated alkyl, substituted or unsubstituted C2-C10 alkenyl, substituted or unsubstituted C2-C10 alkynyl, substituted or unsubstituted C3-C10 cycloalkyl, substituted or unsubstituted C3-C10 cycloalkenyl, substituted or unsubstituted C3-C10 heterocycloalkyl, substituted or unsubstituted C3-C10 heterocycloalkenyl, substituted or unsubstituted aryl, or substituted or unsubstituted heteroaryl; R₂, R₄ and R₅ each is independently H, halogen, —CN, substituted or unsubstituted C1-C10 alkyl, substituted or unsubstituted C2-C10 alkenyl, substituted or unsubstituted C2-C10 alkynyl, substituted or unsubstituted C3-C10 cycloalkyl, substituted or unsubstituted C3-C10 cycloalkenyl, substituted or unsubstituted C3-C10 heterocycloalkyl, substituted or unsubstituted C3-C10 heterocycloalkenyl, substituted or unsubstituted aryl, or substituted or unsubstituted heteroaryl; R₃ is H, D, halogen, —CN, substituted or unsubstituted C1-C10 alkyl, —CD₃, substituted or unsubstituted C2-C10 alkenyl, substituted or unsubstituted C2-C10 alkynyl, substituted or unsubstituted C3-C10 cycloalkyl, substituted or unsubstituted C3-C10 cycloalkenyl, substituted or unsubstituted C3-C10 heterocycloalkyl, substituted or unsubstituted C3-C10 heterocycloalkenyl, substituted or unsubstituted aryl, or substituted or unsubstituted heteroaryl; R₆ is H or D; and m and n each is independently 0, 1, 2 or 3, with the proviso that at least one of m and n is not zero.

The present disclosure is further directed to a method for treating a disease in a subject in need thereof, the method comprising administering to the subject, a therapeutically effective amount of a compound disclosed herein, e.g., a compound of formula (1) or pharmaceutical composition thereof disclosed herein.

### BRIEF DESCRIPTION OF THE FIGURES

FIG. 1. A schematic representation of Formula 1. Waved line represents a group connected to the ring structure at any stereochemical configuration. The bond between position 5 and 6 can be either a single bond (C—C) or a double bond (C═C).

FIG. 2A-FIG. 2F. Representative examples of the compounds having an R₃ group.

FIG. 3A-FIG. 3F. Representative examples of additional compounds wherein the R₃ is —CH₃.

FIG. 4A-FIG. 4F. Representative examples of additional compounds wherein the R₃ is a cycloalkyl.

FIG. 5A-FIG. 5L. Representative examples of the compounds having RX group or a halogen X.

FIG. 6A-FIG. 6F. Representative examples of the compounds wherein the RX group is —CFH₂.

FIG. 7A-FIG. 7L. Representative examples of the compounds having a heterocyclic ring as R₃.

FIG. 8A-FIG. 8F. Representative examples of the compounds having a specific heterocyclic ring as R₃.

### DETAILED DESCRIPTION

Following are more detailed descriptions of various concepts related to, and embodiments of, methods and apparatus according to the present disclosure. It should be appreciated that various aspects of the subject matter introduced above and discussed in greater detail below may be implemented in any of numerous ways, as the subject matter is not limited to any particular manner of implementation. Examples of specific implementations and applications are provided primarily for illustrative purposes.

As used herein, the term “γ aminobutyric acid type A receptors”, “GABA_{A} receptors”, “GABA_{A}Rs”, “GABA_{A}R”, “GABA_{A}Rs”, “GABA_{A}R” or a grammatic variation thereof, either in singular or in plural form, refers to gamma-aminobutyric acid type A receptors (GABA_{A}Rs) that are a class of receptors that respond to the neurotransmitter gamma-aminobutyric acid (GABA). GABA is the principal inhibitory neurotransmitter in the cerebral cortex that is important for maintaining the inhibitory state that counterbalances neuronal excitation. Disorder in GABA_{A} receptors or imbalance of GABA and neuroexcitation can lead to a wide range of brain circuits and disorders related to GABA function that are central to a variety of behavioral states such as anxiety levels, panic, stress response, seizures, sleep, vigilance and memory.

A number of natural and synthetic neuroactive steroids can bind to GABA_{A}Rs and modulate their activities.

As used herein, the term “neuroactive steroid”, “NAS”, “neuroactive steroids”, “NASs” or a variation thereof refers to one or more neurosteroids (NS) that exert inhibitory actions on neurotransmission, specifically, on the GABA_{A} receptors. In some embodiments, neuroactive steroids act as modulators of γ-aminobutyric acid (GABA) receptor complex (GRC) in the central nervous system (CNS). Examples include, but are not limited to, tetrahydrodeoxycorticosterone (THDOC), androstane, androstane 3α-androstanediol, cholestane cholesterol, pregnane, pregnane pregnanolone (eltanolone), allopregnanolone, brexanolone, ganaxolone and SAGE-217.

As used herein, the term “alkyl” refers to a monovalent linear or branched saturated aliphatic carbon chain or radical. For example, “C1-C10 alkyl” (or “C₁-C₁₀ alkyl”) refers to any of alkyls having 1 to 10 carbon atoms, such as —CH₃, —C₂H₅, —C₃H₇, —C₄H₉, —C₅H₁₁, —C₆H₁₃, —C₇H₁₅, —C₈H₁₇, —C₉H₁₉ or —C₁₀H₂₁, that is linear or branched, or of any one of isomers. As another example, “C1-C4 alkyl” refers to n-butyl, i-butyl, s-butyl and t-butyl, n-propyl and i-propyl, ethyl or methyl.

As used herein, the term “alkylene” or “alkylene chain” refers to a fully saturated, straight or branched divalent hydrocarbon chain radical, and having from one to twelve carbon atoms. Non-limiting examples of C1-C10 alkylene include methylene, ethylene, propylene, n-butylene, and the like. The alkylene chain can be attached to the rest of the molecule through a single bond and to a radical group (e.g., those described herein) through a single bond. The points of attachment of the alkylene chain to the rest of the molecule and to the radical group can be through one carbon or any two carbons within the chain. Unless stated otherwise specifically in the specification, an alkylene chain can be optionally substituted.

As used herein, the term “alkenyl” refers to a linear or branched chain aliphatic hydrocarbon radical containing at least one carbon-carbon double bond and having a number of carbon atoms in the specified range. For example, “C2-C10 alkenyl” (or “C₂-C₁₀ alkenyl”) refers to any of alkenyls having 2 to 6 carbon atoms that is linear or branched, or isomers. In another example C2-C10 alkenyl can refer to 1-butenyl, 2-butenyl, 3-butenyl, isobutenyl, 1-propenyl, 2-propenyl, or ethenyl (or vinyl).

As used herein, the term “alkynyl” refers to a radical of a straight-chain or branched hydrocarbon group having at least one or more carbon-carbon triple bonds, and optionally, one or more carbon-carbon double bonds. In some examples, a C2-C10 alkynyl can have in a range of from 2 to 10 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds). In certain examples, alkynyl can contain no double bonds. In some examples, an alkynyl group has 2 to 10 carbon atoms (“C2-C10 alkynyl”). In yet some examples, an alkynyl group has 2 to 9 carbon atoms (“C2-C9 alkynyl”). In yet some examples, an alkynyl group has 2 to 8 carbon atoms (“C2-C8 alkynyl”). In yet some examples, an alkynyl group has 2 to 7 carbon atoms (“C2-C7 alkynyl”). In yet some examples, an alkynyl group has 2 to 6 carbon atoms (“C2-C6 alkynyl”). In yet some examples, an alkynyl group has 2 to 5 carbon atoms (“C2-C5 alkynyl”). In yet some examples, an alkynyl group has 2 to 4 carbon atoms (“C2-C4 alkynyl”). In yet some examples, an alkynyl group has 2 to 3 carbon atoms (“C2-C3 alkynyl”). In yet some examples, an alkynyl group has 2 carbon atoms (“C2 alkynyl”). The one or more carbon-carbon triple bonds can be internal (such as in 2-butynyl) or terminal (such as in 1-butynyl). Examples of C2-C4 alkynyl groups include, without limitation, ethynyl (C2), 1-propynyl (C3), 2-propynyl (C3), 1-butynyl (C4), 2-butynyl (C4), and the like. Examples of C2-C6 alkynyl groups can include the aforementioned C2-C4 alkynyl groups as well as pentynyl (C5), hexynyl (C6), and the like. Additional examples of alkynyl can include heptynyl (C7), octynyl (C8), and the like. Unless otherwise specified, each instance of an alkynyl group can be independently and, optionally, substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is an unsubstituted C2-C10 alkynyl. In certain embodiments, the alkynyl group is a substituted C2-C10 alkynyl.

As used herein, the term “cycloalkyl” refers to any monocyclic ring of an alkane having a number of carbon atoms in the specified range. For example, “C3-C10 cycloalkyl” (or “C₃-C₁₀ cycloalkyl”) refers to monocyclic ring of an alkane having 3 to 6 carbon atoms, such as cyclopropyl, cyclobutyl, cyclopentyl, cyclohexyl, and cycloheptyl.

As used herein, the term “heterocycloalkyl”, “heterocycloalkenyl” or “heterocyclic ring” refers to a ring structure having carbon atoms and one or more heteroatoms selected from N, O, S, boron, silicon, phosphorus or a combination thereof, as members of the ring structure. In some embodiments, the “heterocyclalkyl is a “C3-C10 heterocycloalkyl” (or C₃-C₁₀ heterocycloalkyl,) that comprises one or more heteroatoms, such as N, O, S or a combination thereof. A heterocycloalkyl can have one or more substitutions. The substitutions can be on one or more carbon atoms or any of the heteroatoms.

As used herein, the term “haloalkyl” refers to an alkyl group, as defined above, that is substituted with one or more halogen atoms (e.g., F, Cl, Br, and I). When a haloalkyl comprises two or more halogen atoms, the halogen atoms can be the same or different. Non-limiting examples of haloalkyl groups include trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

As used herein, the term “heteroalkyl” refers to an alkyl group that further comprises 1 or more heteroatoms (e.g., N, O, S, boron, silicon, phosphorus or a combination thereof) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain examples, a heteroalkyl group refers to a saturated group having from 1 to 10 carbon atoms and one or more heteroatoms (“hetero C1-C10 alkyl”). In some other examples, a heteroalkyl group is a saturated group having 1 to 9 carbon atoms and one or more heteroatoms (“hetero C1-C9 alkyl”). In further examples, a heteroalkyl group is a saturated group having 1 to 8 carbon atoms and one or more heteroatoms (“hetero C1-C8 alkyl”). In yet further examples, a heteroalkyl group is a saturated group having 1 to 7 carbon atoms and one or more heteroatoms (“hetero C1-C7 alkyl”). In yet further examples, a heteroalkyl group is a group having 1 to 6 carbon atoms and one or more heteroatoms (“hetero C1-C6 alkyl”). In yet further examples, a heteroalkyl group is a saturated group having 1 to 5 carbon atoms and 1 or more heteroatoms (“hetero C1-C5 alkyl”). In yet further examples, a heteroalkyl group is a saturated group having 1 to 4 carbon atoms and 1 or 2 heteroatoms (“hetero C1-C4 alkyl”). In yet further examples, a heteroalkyl group is a saturated group having 1 to 3 carbon atoms and 1 heteroatom (“hetero C1-C3 alkyl”). In yet further examples, a heteroalkyl group is a saturated group having 1 to 2 carbon atoms and 1 heteroatom (“hetero C1-C2 alkyl”). In yet further examples, a heteroalkyl group is a saturated group having 1 carbon atom and 1 heteroatom (“hetero C1 alkyl”). In yet further examples, a heteroalkyl group is a saturated group having 2 to 6 carbon atoms and 1 or 2 heteroatoms (“hetero C2-C6 alkyl”). Unless otherwise specified, each instance of a heteroalkyl group is independently unsubstituted (an “unsubstituted heteroalkyl”) or substituted (a “substituted heteroalkyl”) with one or more substituents. In yet further examples, the heteroalkyl group is an unsubstituted hetero C1-C10 alkyl. In even further examples, the heteroalkyl group is a substituted hetero C1-C10 alkyl. The substitutions can be on one or more carbon atoms or any of the heteroatoms.

As used herein, the term “heteroalkenyl” refers to an alkenyl group further comprises one or more (e.g., 1, 2, 3, or 4) heteroatoms (e.g., N, O, S, boron, silicon, phosphorus or a combination thereof) wherein the one or more heteroatoms is inserted between adjacent carbon atoms within the parent carbon chain and/or one or more heteroatoms is inserted between a carbon atom and the parent molecule, i.e., between the point of attachment. In some examples, a heteroalkenyl group refers to a group having from 2 to 10 carbon atoms, at least one double bond, and 1, 2, 3, or 4 heteroatoms (“hetero C2-C10 alkenyl”). In further examples, a heteroalkenyl group has 2 to 9 carbon atoms at least one double bond, and 1, 2, 3, or 4 heteroatoms (“hetero C2-C9 alkenyl”). In yet further examples, a heteroalkenyl group has 2 to 8 carbon atoms, at least one double bond, and 1, 2, 3, or 4 heteroatoms (“hetero C2-C8 alkenyl”). In yet further examples, a heteroalkenyl group has 2 to 7 carbon atoms, at least one double bond, and 1, 2, 3, or 4 heteroatoms (“hetero C2-C7 alkenyl”). In yet further examples, a heteroalkenyl group has 2 to 6 carbon atoms, at least one double bond, and 1, 2, or 3 heteroatoms (“hetero C2-C6 alkenyl”). In yet further examples, a heteroalkenyl group has 2 to 5 carbon atoms, at least one double bond, and 1 or 2 heteroatoms (“hetero C2-C5 alkenyl”). In yet further examples, a heteroalkenyl group has 2 to 4 carbon atoms, at least one double bond, and 1 or 2 heteroatoms (“hetero C2-C4 alkenyl”). In yet further examples, a heteroalkenyl group has 2 to 3 carbon atoms, at least one double bond, and 1 heteroatom (“hetero C2-C3 alkenyl”). In yet further examples, a heteroalkenyl group has 2 to 6 carbon atoms, at least one double bond, and 1 or 2 heteroatoms (“hetero C2-C6 alkenyl”). Unless otherwise specified, each instance of a heteroalkenyl group is independently unsubstituted (an “unsubstituted heteroalkenyl”) or substituted (a “substituted heteroalkenyl”) with one or more substituents. In yet further examples, the heteroalkenyl group is an unsubstituted hetero C2-C10 alkenyl. In even further examples, the heteroalkenyl group is a substituted hetero C2-C10 alkenyl. The substitutions can be on one or more carbon atoms or any of the heteroatoms.

As used herein, the term “heteroalkynyl” refers to an alkynyl group further comprises one or more heteroatoms (e.g., N, O, S, boron, silicon, phosphorus or a combination thereof), wherein the one or more heteroatoms is inserted between adjacent carbon atoms within the parent carbon chain and/or one or more heteroatoms is inserted between a carbon atom and the parent molecule, i.e., between the point of attachment. In certain examples, a heteroalkynyl group refers to a group having from 2 to 10 carbon atoms, at least one triple bond, and 1, 2, 3, or 4 heteroatoms (“hetero C2-C10 alkynyl”). In further examples, a heteroalkynyl group has 2 to 9 carbon atoms, at least one triple bond, and 1, 2, 3, or 4 heteroatoms (“hetero C2-C9 alkynyl”). In yet further examples, a heteroalkynyl group has 2 to 8 carbon atoms, at least one triple bond, and 1, 2, 3, or 4 heteroatoms (“hetero C2-C8 alkynyl”). In yet further examples, a heteroalkynyl group has 2 to 7 carbon atoms, at least one triple bond, and 1, 2, 3, or 4 heteroatoms (“hetero C2-C7 alkynyl”). In yet further examples, a heteroalkynyl group has 2 to 6 carbon atoms, at least one triple bond, and 1, 2, or 3 heteroatoms (“hetero C2-C6 alkynyl”). In yet further examples, a heteroalkynyl group has 2 to 5 carbon atoms, at least one triple bond, and 1 or 2 heteroatoms (“hetero C2-C5 alkynyl”). In yet further examples, a heteroalkynyl group has 2 to 4 carbon atoms, at least one triple bond, and 1 or 2 heteroatoms (“hetero C2-C4 alkynyl”). In yet further examples, a heteroalkynyl group has 2 to 3 carbon atoms, at least one triple bond, and 1 heteroatom (“hetero C2-C3 alkynyl”). In yet further examples, a heteroalkynyl group has 2 to 6 carbon atoms, at least one triple bond, and 1 or 2 heteroatoms (“hetero C2-C6 alkynyl”). Unless otherwise specified, each instance of a heteroalkynyl group is independently unsubstituted (an “unsubstituted heteroalkynyl”) or substituted (a “substituted heteroalkynyl”) with one or more substituents. In yet further examples, the heteroalkynyl group is an unsubstituted hetero C2-C10 alkynyl. In even further examples, the heteroalkynyl group is a substituted hetero C2-C10 alkynyl. The substitutions can be on one or more carbon atoms or any of the heteroatoms.

As used herein, the term “cycloalkylalkyl” refers to an alkyl radical in which the alkyl group is substituted with a cycloalkyl group. Typical cycloalkylalkyl groups can include, but are not limited to, cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, and cyclooctylethyl, and the like. The cycloalkylalkyl can be unsubstituted or substituted. The substitutions can be on one or more carbon atoms or any of the heteroatoms.

As used herein, the term “heterocyclylalkyl” refers to an alkyl radical in which the alkyl group is substituted with a heterocyclyl group. Typical heterocyclylalkyl groups include, but are not limited to, pyrrolidinylmethyl, piperidinylmethyl, piperazinylmethyl, morpholinylmethyl, pyrrolidinylethyl, piperidinylethyl, piperazinylethyl, morpholinylethyl, and the like. The heterocyclylalkyl can be unsubstituted or substituted. The substitutions can be on one or more carbon atoms or any of the heteroatoms.

As used herein, the term “cycloalkenyl” refers to substituted or unsubstituted carbocyclyl group having from 3 to 10 carbon atoms and having a single cyclic ring or multiple condensed rings, including fused and bridged ring systems and having at least one and particularly from 1 to 2 sites of olefinic unsaturation. Such cycloalkenyl groups include, by way of example, single ring structures such as cyclohexenyl, cyclopentenyl, cyclopropenyl, and the like. The cycloalkenyl can be unsubstituted or substituted. The substitutions can be on one or more carbon atoms or any of the heteroatoms.

As used herein, the term “aryl” refers to a cyclic or one or more fused cyclic hydrocarbon ring systems in which at least one ring is aromatic. The term “heteroaryl” refers to heteroaromatic ring, that contains one or more, such as in a range of from 1 to 4 heteroatoms independently selected from N, O and S, wherein each N is optionally in the form of an oxide to the extent chemically possible. The aryl or heteroaryl can be substituted or unsubstituted. The substitutions can be on one or more carbon atoms or any of the heteroatoms.

As used herein, the term “halogen” (or “halo”) refers to fluorine, chlorine, bromine, and iodine (alternatively, referred to as fluoro (—F), chloro (—Cl), bromo (—Br), and iodo (—I)).

As used herein, the term “isomer” refers to a structural isomer, such as a group or an atom positioned at different locations of a molecule; stereoisomer, such as a chiral isomer, enantiomer, diastereomer and cis/trans isomer; a tautomer; or a combination thereof. A mixture of isomers can also be suitable. A mixture of isomers can comprise the respective isomers in all ratios. A salt of an isomer can also be suitable. A neuroactive steroid of this invention can comprise isomers thereof, one or more salts thereof, one or more solvates including hydrates thereof, solvated salts thereof or a mixture thereof. Absolute stereochemistry or isomer configuration may be determined by X-ray crystallography, by Vibrational Circular Dichroism (VCD) spectroscopy analysis or a combination thereof. An isomer that can have desired biological activity in vivo can be particularly preferred.

The neuroactive steroids disclosed herein can be identified by names based on the nomenclature recommended by International Union of Pure and Applied Chemistry (IUPAC) or other nomenclature systems. These compounds can also be identified by chemical structure drawings. Unless expressly stated to the contrary in a particular context, the names and the structures may be used interchangeably throughout this disclosure.

As used herein, “an “effective amount” refers to a therapeutically effective amount or a prophylactically effective amount. A “therapeutically effective amount” refers to an amount effective, at dosages and for periods of time necessary, to achieve the desired therapeutic result, such as reduced tumor size, increased life span or increased life expectancy. A therapeutically effective amount of a compound can vary according to factors such as the disease state, age, sex, and weight of the subject, and the ability of the compound to elicit a desired response in the subject. Dosage regimens can be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as smaller tumors, increased life span, increased life expectancy or prevention of the progression of prostate cancer to a castration-resistant form. Typically, a prophylactic dose is used in subjects prior to or at an earlier stage of disease, so that a prophylactically effective amount can be less than a therapeutically effective amount.

As used herein, “treating” or “treatment” covers the treatment of the disease or condition of interest in a mammal, for example in a human, having the disease or condition of interest, and includes (but is not limited to):

1. preventing the disease or condition from occurring in a mammal, in particular, when such mammal is predisposed to the condition but has not yet been diagnosed as having it;
2. inhibiting the disease or condition, i.e., arresting its development;
3. relieving the disease or condition, i.e., causing regression of the disease or condition (ranging from reducing the severity of the disease or condition to curing the disease of condition); or
4. relieving the symptoms resulting from the disease or condition, i.e., relieving pain without addressing the underlying disease or condition.

As used herein, the terms “disease” and “condition” can be used interchangeably or can be different in that the particular malady or condition cannot have a known causative agent (so that etiology has not yet been worked out) and it is therefore not yet recognized as a disease, but only as an undesirable condition or syndrome, wherein a more or less specific set of symptoms have been identified by clinicians.

As used herein, a “subject” can be a human, non-human primate, mammal, rat, mouse, cow, horse, pig, sheep, goat, dog, cat, insect and the like. The subject can be suspected of having or at risk for having a cancer, such as a blood cancer, or another disease or condition. Diagnostic methods for various cancers, and the clinical delineation of cancer, are known to those of ordinary skill in the art. The subject can also be suspected of having an infection or abnormal cardiovascular function.

Unless expressly stated to the contrary, all ranges cited herein are inclusive. For example, a heterocyclic ring described as comprising in a range of from “1 to 4 heteroatoms” means the ring can comprise 1, 2, 3 or 4 heteroatoms. It is also to be understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, 3 heteroatoms, or 4 heteroatoms. In other examples, C1-C10 alkyl means an alkyl comprises 1, 2, 3, 4, 5, 6, 7, 8, 9 and 10 carbon atoms including all sub-ranges. Thus, a C1-C10 alkyl can be a methyl, ethyl, propyl, C4 alkyl, C5 alkyl, C6 alkyl, C7 alkyl, C8 alkyl, C9 alkyl and C10 alkyl. Additionally, each C1-C10 alkyl can be independently linear or branched. Similarly, C2-C10 alkenyl means an alkenyl comprises 2, 3, 4, 5, 6, 7, 8, 9 and 10 carbon atoms, linear or branched. A linear or a branched alkenyl can be suitable. A C3-C10 cycloalkyl means a cycloalkyl comprises 3, 4, 5, 6, 7, 8, 9 and 10 carbon atoms, linear or branched.

The present disclosure is directed to a neuroactive steroid (NAS) of formula (1):

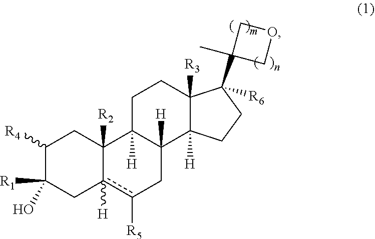

one or more isomers thereof, a deuterium labeled variant thereof, or a pharmaceutically acceptable salt thereof,
wherein:
R₁ is H, D, substituted or unsubstituted C1-C10 alkyl, C1-C5 deuterated alkyl, substituted or unsubstituted C2-C10 alkenyl, substituted or unsubstituted C2-C10 alkynyl, substituted or unsubstituted C3-C10 cycloalkyl, substituted or unsubstituted C3-C10 cycloalkenyl, substituted or unsubstituted C3-C10 heterocycloalkyl, substituted or unsubstituted C3-C10 heterocycloalkenyl, substituted or unsubstituted aryl, or substituted or unsubstituted heteroaryl;
R₂, R₄ and R₅ each is independently H, halogen, —CN, substituted or unsubstituted C1-C10 alkyl, substituted or unsubstituted C2-C10 alkenyl, substituted or unsubstituted C2-C10 alkynyl, substituted or unsubstituted C3-C10 cycloalkyl, substituted or unsubstituted C3-C10 cycloalkenyl, substituted or unsubstituted C3-C10 heterocycloalkyl, substituted or unsubstituted C3-C10 heterocycloalkenyl, substituted or unsubstituted aryl, substituted or unsubstituted heteroaryl;
R₃ is H, D, halogen, —CN, substituted or unsubstituted C1-C10 alkyl, —CD₃, substituted or unsubstituted C2-C10 alkenyl, substituted or unsubstituted C2-C10 alkynyl, substituted or unsubstituted C3-C10 cycloalkyl, substituted or unsubstituted C3-C10 cycloalkenyl, substituted or unsubstituted C3-C10 heterocycloalkyl, substituted or unsubstituted C3-C10 heterocycloalkenyl, substituted or unsubstituted aryl, or substituted or unsubstituted heteroaryl;

### R₆ is H or D; and

m and n each is independently an integer 0, 1, 2 or 3, with the proviso that at least one of m and n is not zero.

Any of the atoms in a compound disclosed herein may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds disclosed herein.

In some embodiments, the compounds of the present disclosure include all isotopes of atoms occurring in the intermediates or final compounds. Isotopes include those atoms having the same atomic number but different mass numbers. For example, isotopes of hydrogen include tritium and deuterium. In some embodiments, a compound disclosed herein, e.g., compounds of formula (1), includes one or more deuterium atoms. In some embodiments, one or more of R₁, R₂, R₃, R₄, and R₅ is a deuterium atom or a deuterated alkyl group (e.g. a C1-C10 deuterated alkyl group or a C1-C5 deuterated alkyl group). In some embodiments, one or more of R₁, R₂, R₃, R₄, and R₅ is a deuterium atom or —CD₃. In some embodiments of formula (1), R₃ is a deuterium atom or a deuterated alkyl group, and one or more C—H bonds of formula (1) are replaced with a C-D bond.

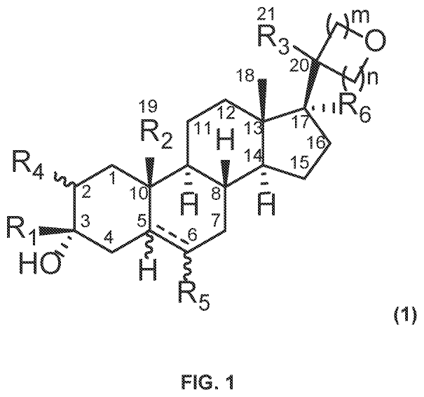

A schematic representation of compounds of Formula (1) is shown in FIG. 1, wherein positions of carbon atoms are indicated by numbers. A waved line in FIG. 1 represents a group connected to the ring structure at a chiral center, if a chiral center is present at the position. The bond between positions 5 and 6, as represented by a pair of solid and dotted lines, can be either a single bond (C—C) or a double bond (C═C). In some embodiments, the bond between positions 5 and 6 is a C—C bond. In some embodiments, the bond between positions 5 and 6 is a C═C double bond.

In some embodiments of formula (1), R₁ is H, D, substituted or unsubstituted C1-C10 alkyl, —CD₃, substituted or unsubstituted C2-C10 alkenyl, substituted or unsubstituted C2-C10 alkynyl, substituted or unsubstituted C3-C10 cycloalkyl, substituted or unsubstituted C3-C10 cycloalkenyl, substituted or unsubstituted C3-C10 heterocycloalkyl, substituted or unsubstituted C3-C10 heterocycloalkenyl, substituted or unsubstituted aryl, or substituted or unsubstituted heteroaryl. In some embodiments, R₁ is H, substituted or unsubstituted C1-C10 alkyl, substituted or unsubstituted C2-C10 alkenyl, substituted or unsubstituted C2-C10 alkynyl, substituted or unsubstituted C3-C10 cycloalkyl, substituted or unsubstituted C3-C10 cycloalkenyl, substituted or unsubstituted C3-C10 heterocycloalkyl, substituted or unsubstituted C3-C10 heterocycloalkenyl, substituted or unsubstituted aryl, or substituted or unsubstituted heteroaryl. In some embodiments, R₁ is H, substituted or unsubstituted alkyl, substituted or unsubstituted alkenyl, substituted or unsubstituted alkynyl, or substituted or unsubstituted cycloalkyl. In some embodiments, R₁ is independently H, D, substituted or unsubstituted alkyl, —CD₃, or substituted or unsubstituted cycloalkyl. In some embodiments, R₁ is independently H, substituted or unsubstituted alkyl, or substituted or unsubstituted cycloalkyl. In some embodiments, R₁ is independently H, D, substituted or unsubstituted alkyl, or —CD₃. In some embodiments, R₁ is independently H or substituted or unsubstituted alkyl. In some embodiments, R₁ is independently H or unsubstituted alkyl. In some embodiments, R₁ is H. In some embodiments, R₁ is substituted or unsubstituted alkyl. In some embodiments, R₁ is unsubstituted alkyl. In some embodiments, the alkyl is a C1-C10 alkyl. In some embodiments, the alkyl is a C1-C5 alkyl. In some embodiments, the alkyl is methyl, ethyl, or isopropyl. In some embodiments, the alkyl is methyl or ethyl. In some embodiments, the alkyl is methyl. In some embodiments, the alkenyl is a C2-C10 alkenyl. In some embodiments, the alkenyl is a C2-C5 alkenyl. In some embodiments, the alkynyl is a C2-C10 alkynyl. In some embodiments, the alkynyl is a C2-C5 alkynyl. In some embodiments, the cycloalkyl is a C3-C10 cycloalkyl. In some embodiments, the cycloalkyl is a C3-C6 cycloalkyl. In some embodiments, the cycloalkyl is a cyclopropyl or cyclobutyl. In some embodiments, the cycloalkyl is a cyclopropyl.

In some embodiments of formula (1), R₂, R₄ and R₅ each is independently H, halogen, —CN, substituted or unsubstituted C1-C5 alkyl, substituted or unsubstituted C2-C6 alkenyl, substituted or unsubstituted C2-C6 alkynyl, substituted or unsubstituted C3-C6 cycloalkyl, substituted or unsubstituted C3-C6 cycloalkenyl, substituted or unsubstituted C3-C6 heterocycloalkyl, substituted or unsubstituted C3-C6 heterocycloalkenyl, substituted or unsubstituted aryl, or substituted or unsubstituted heteroaryl. In some embodiments, R₂, R₄ and R₅ each is independently H, halogen, —CN, substituted or unsubstituted C1-C5 alkyl or C3-C6 cycloalkyl. In some embodiments, R₂, R₄ and R₅ each is independently H, halogen, —CN, or substituted or unsubstituted C1-C5 alkyl. In some embodiments, R₂, R₄ and R₅ each is independently H, halogen, or substituted or unsubstituted C1-C5 alkyl. In some embodiments, the C1-C5 alkyl is methyl or ethyl. In some embodiments, the C1-C5 alkyl is methyl. In some embodiments, the C2-C6 alkenyl is ethenyl, propenyl, or isopropenyl. In some embodiments, the substituted or unsubstituted C2-C6 alkynyl is substituted or unsubstituted ethynyl, propynyl, or butynyl. In some embodiments, the substituted or unsubstituted C3-C6 cycloalkyl is substituted or unsubstituted cyclopropyl or cyclobutyl. In some embodiments, the substituted or unsubstituted C3-C6 cycloalkyl is substituted or unsubstituted cyclopropyl. In some embodiments, the substituted or unsubstituted C3-C6 heterocycloalkyl is substituted or unsubstituted azetidinyl, pyrrolidinyl, piperidinyl, morpholinyl, or thiomorpholinyl. In some embodiments, the substituted or unsubstituted aryl is substituted or unsubstituted phenyl. In some embodiments, the substituted or unsubstituted heteroaryl is substituted or unsubstituted oxazolyl, thiazolyl, imidazolyl, triazolyl, pyrazolyl, isoxazolyl, oxadiazolyl, thiadiazolyl, pyridinyl, or pyrimidinyl. In some embodiments, R₂, R₄ and R₅ each is independently H, halogen, —CN, —CH₃, —CH₂CH₃, —CH₂CH₂CH₃, —CH(CH₃)₂, —CH═CH₂, —C(Me)=CH₂, —CH═CH(Me), —C≡CH, —C≡C(Me), cyclopropyl, cyclobutyl, phenyl, or pyridinyl.

In some embodiments of formula (1), R₃ is H, D, halogen, —CN, substituted or unsubstituted C1-C5 alkyl, deuterated C1-C5 alkyl, substituted or unsubstituted C2-C6 alkenyl, substituted or unsubstituted C2-C6 alkynyl, substituted or unsubstituted C3-C6 cycloalkyl, substituted or unsubstituted C3-C6 cycloalkenyl, substituted or unsubstituted C3-C6 heterocycloalkyl, substituted or unsubstituted C3-C6 heterocycloalkenyl, substituted or unsubstituted aryl, or substituted or unsubstituted heteroaryl. In some embodiments, R₃ is H, D, halogen, —CN, substituted or unsubstituted C1-C5 alkyl, —CD₃, substituted or unsubstituted C2-C6 alkenyl, substituted or unsubstituted C2-C6 alkynyl, substituted or unsubstituted C3-C6 cycloalkyl, substituted or unsubstituted C3-C6 cycloalkenyl, substituted or unsubstituted C3-C6 heterocycloalkyl, substituted or unsubstituted C3-C6 heterocycloalkenyl, substituted or unsubstituted aryl, or substituted or unsubstituted heteroaryl. In some embodiments, R₃ is H, halogen, —CN, substituted or unsubstituted C1-C5 alkyl, substituted or unsubstituted C2-C6 alkenyl, substituted or unsubstituted C2-C6 alkynyl, substituted or unsubstituted C3-C6 cycloalkyl, substituted or unsubstituted C3-C6 cycloalkenyl, substituted or unsubstituted C3-C6 heterocycloalkyl, substituted or unsubstituted C3-C6 heterocycloalkenyl, substituted or unsubstituted aryl, or substituted or unsubstituted heteroaryl. In some embodiments, R₃ is H, D, halogen, —CN, substituted or unsubstituted C1-C5 alkyl, deuterated C1-C5 alkyl, or C3-C6 cycloalkyl. In some embodiments, R₃ is H, D, halogen, —CN, substituted or unsubstituted C1-C5 alkyl, —CD₃, or C3-C6 cycloalkyl. In some embodiments, R₃ is H, halogen, —CN, substituted or unsubstituted C1-C5 alkyl or C3-C6 cycloalkyl. In some embodiments, R₃ is H, D, halogen, —CN, substituted or unsubstituted C1-C5 alkyl, —CD₃. In some embodiments, R₃ is H, halogen, —CN, or substituted or unsubstituted C1-C5 alkyl. In some embodiments, R₃ is independently H, halogen, or substituted or unsubstituted C1-C5 alkyl. In some embodiments, the C1-C5 alkyl is methyl or ethyl. In some embodiments, the C1-C5 alkyl is methyl. In some embodiments, the halogen is F. In some embodiments, the C2-C6 alkenyl is ethenyl, propenyl, or isopropenyl. In some embodiments, the substituted or unsubstituted C2-C6 alkynyl is substituted or unsubstituted ethynyl, propynyl, or butynyl. In some embodiments, the substituted or unsubstituted C3-C6 cycloalkyl is substituted or unsubstituted cyclopropyl or cyclobutyl. In some embodiments, the substituted or unsubstituted C3-C6 cycloalkyl is substituted or unsubstituted cyclopropyl. In some embodiments, the substituted or unsubstituted C3-C6 heterocycloalkyl is substituted or unsubstituted azetidinyl, pyrrolidinyl, piperidinyl, morpholinyl, or thiomorpholinyl. In some embodiments, the substituted or unsubstituted aryl is substituted or unsubstituted phenyl. In some embodiments, the substituted or unsubstituted heteroaryl is substituted or unsubstituted oxazolyl, thiazolyl, imidazolyl, triazolyl, pyrazolyl, isoxazolyl, oxadiazolyl, thiadiazolyl, pyridinyl, or pyrimidinyl. In some embodiments, R₃ is H, halogen, —CN, —CH₃, —CH₂CH₃, —CH₂CH₂CH₃, —CH(CH₃)₂, —CH═CH₂, —C(Me)=CH₂, —CH═CH(Me), —C≡CH, —C≡C(Me), cyclopropyl, cyclobutyl, phenyl, or pyridinyl.

In some embodiments of formula (1), R₃ is H, halogen, substituted or unsubstituted C1-C10 alkyl, substituted or unsubstituted C2-C10 alkenyl, substituted or unsubstituted C2-C10 alkynyl, substituted or unsubstituted C3-C10 cycloalkyl, substituted or unsubstituted C3-C10 cycloalkenyl, substituted or unsubstituted C3-C10 heterocycloalkyl, substituted or unsubstituted C3-C10 heterocycloalkenyl, substituted or unsubstituted aryl, or substituted or unsubstituted heteroaryl. Any of the aforementioned substituted or unsubstituted C1-C10 alkyl, substituted or unsubstituted C2-C10 alkenyl, substituted or unsubstituted C2-C10 alkynyl, substituted or unsubstituted C3-C10 cycloalkyl and substituted or unsubstituted C3-C10 cycloalkenyl, substituted or unsubstituted aryl, substituted or unsubstituted heteroaryl can be suitable.

In some embodiments, R₃ is H, -D, —CH₃, —CD₃, —CN, substituted or unsubstituted cyclopropyl, substituted or unsubstituted C1-C10 haloalkyl, substituted or unsubstituted C3-C10 heterocycloalkyl, substituted or unsubstituted C3-C10 heterocycloalkenyl, substituted or unsubstituted aryl, substituted or unsubstituted heteroaryl, —X, or

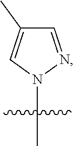

wherein X is selected from the group consisting of Cl, F, Br and I.

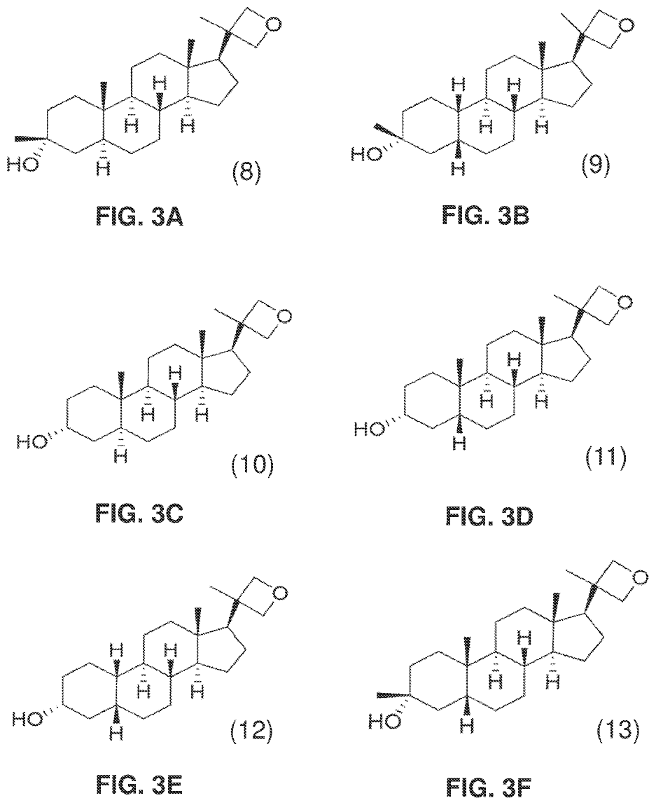

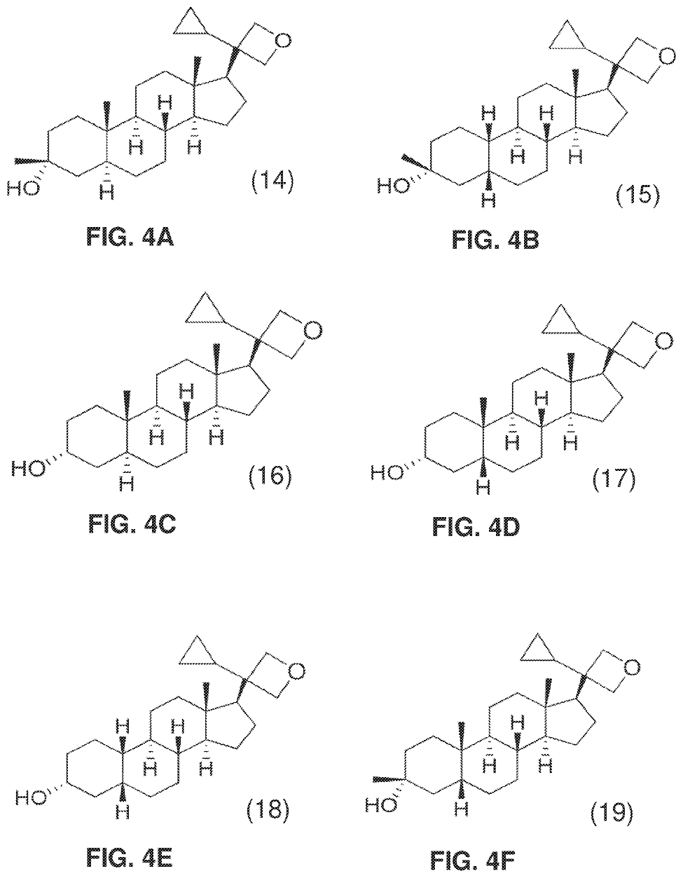

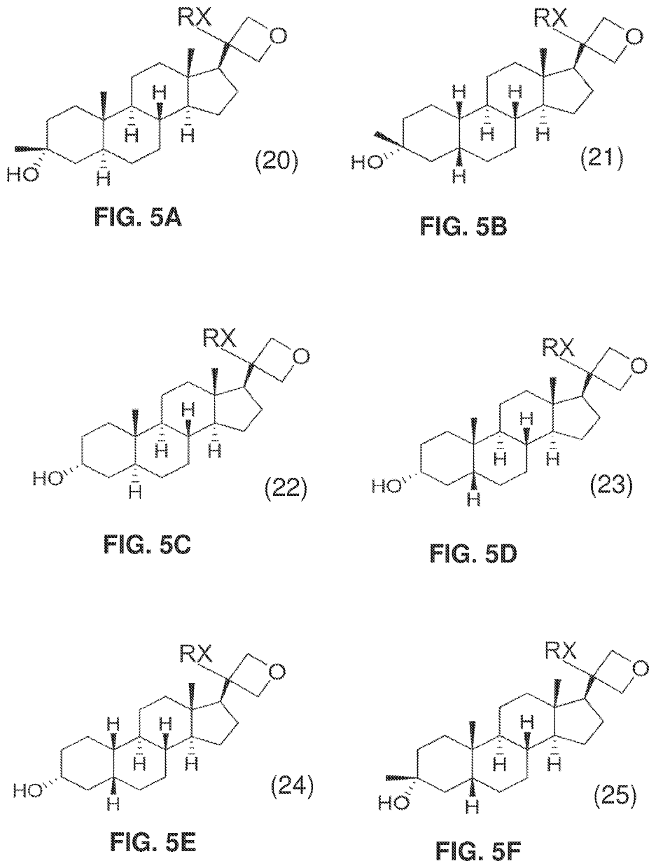

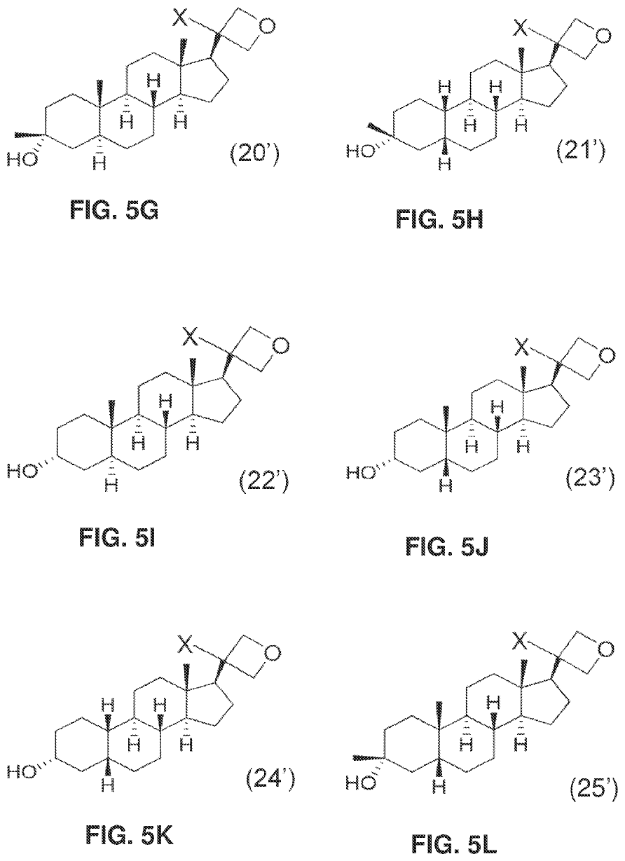

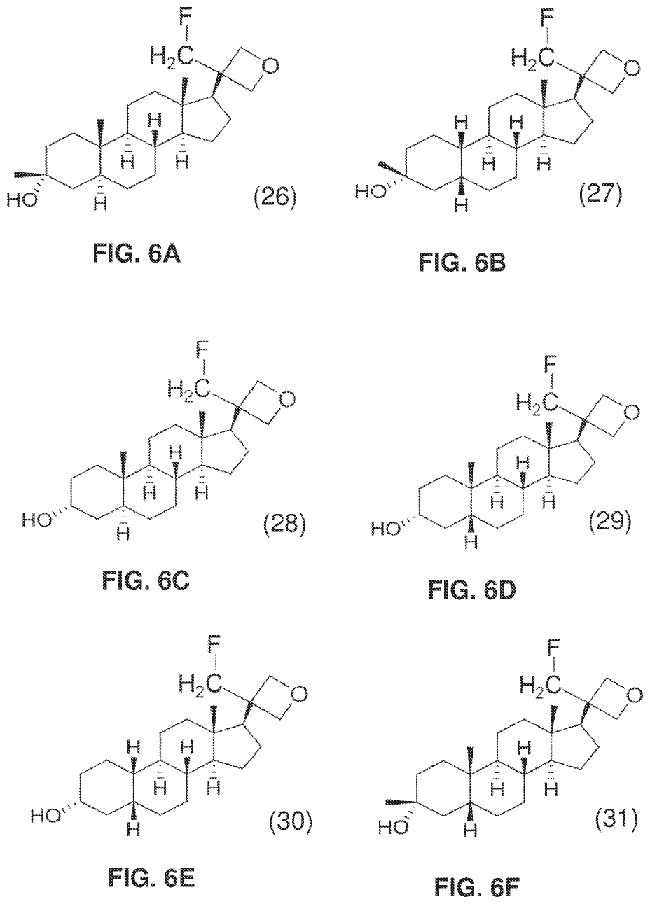

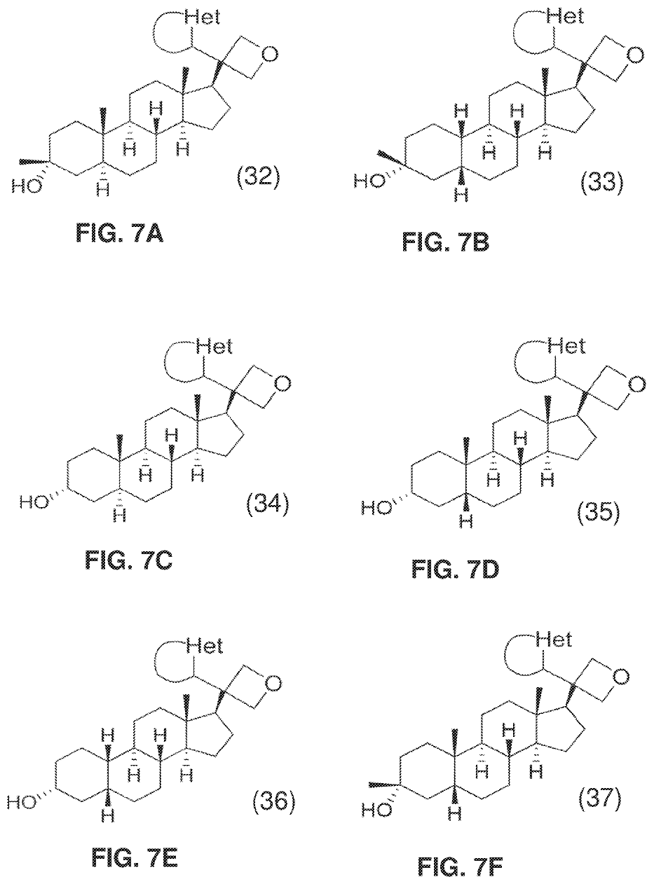

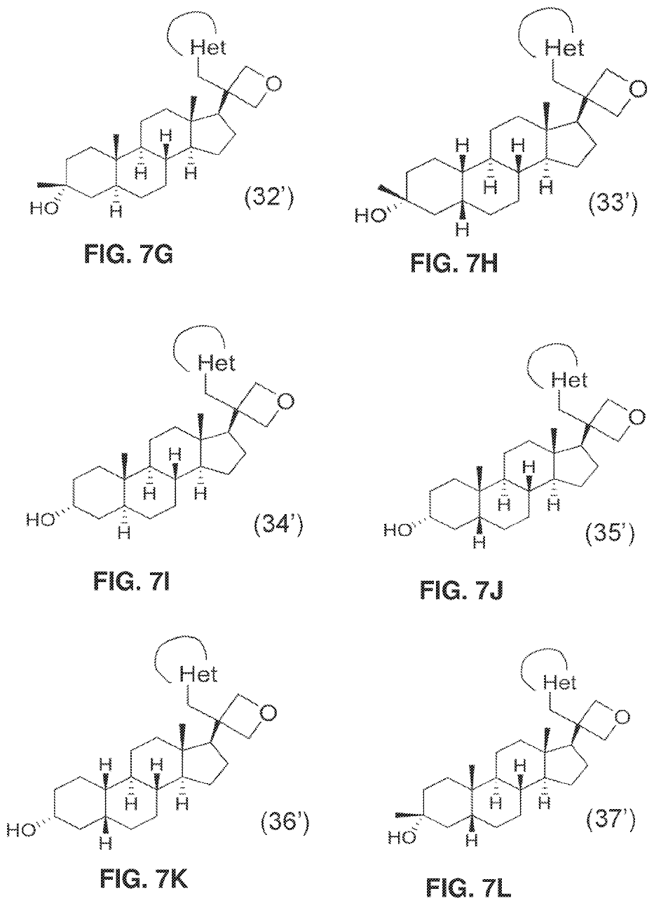

In some embodiments, R₃ is —CH₃ (e.g., as in formulas (8)-(13) of FIG. 3A-FIG. 3F), a substituted or unsubstituted cyclopropyl (e.g., as in formulas (14)-(19) of FIG. 4A-FIG. 4F), a substituted or unsubstituted C3-C10 heterocycloalkyl, a substituted or unsubstituted C3-C10 heterocycloalkenyl (e.g., as in formulas (32)-(37) and (32′)-(37′) of FIG. 7A-FIG. 7L), a halogen (e.g., as in formulas (20′)-(25′) of FIG. 5G-5L, wherein the halogen is represented by X) or a substituted or unsubstituted C1-C10 haloalkyl (e.g., as in formulas (20)-(25) of FIG. 5A-FIG. 5F, where the C1-C10 halogen is represented by RX). In some embodiments, the halogen is Cl, F, Br or I. In some embodiments, the halogen is Cl or F. In some embodiments, the halogen is Br or I. In some embodiments, the halogen is C1. In some embodiments, the halogen is F. In some embodiments, the halogen is Br. In some embodiments, the halogen is I. In some embodiments, R₃ is a C1-C10 haloalkyl. In some embodiment, the C1-C10 haloalkyl is —CXH₂, —CX₂H, —CX₃, —CH₂CXH₂, —CH₂CX₂H, or —CH₂CX₃, wherein X is Cl, F, Br, or I. In some embodiments, the C1-C10 haloalkyl is —CClH₂, —CCl₂H, —CCl₃, —CFH₂, —CF₂H, —CF₃, —CBrH₂, —CBr₂H, —CBr₃, —CIH₂, —Cl₂H, —CI₃, —CClFH, —CClBrH, —CCl(I)H, —CFBrH, —CF(I)H, —CBr(I)H, —CCl₂F, —CClF₂, —CCl₂Br, —CClBr₂, —CCl₂(I), —CCl(I)₂, —CF₂Br, —CFBr₂, —CF₂(I), —CF(I)₂, and the like (some of the iodine is shown as (I) for illustration purposes). In some embodiments, the C1-C10 haloalkyl is —CFH₂ (e.g., formulas (26)-(31) of FIG. 6A-FIG. 6F), —CF₂H, —CF₃, CH₂CFH₂, —CH₂CF₂H or —CH₂CF₃.

In some embodiments, R₃ is:

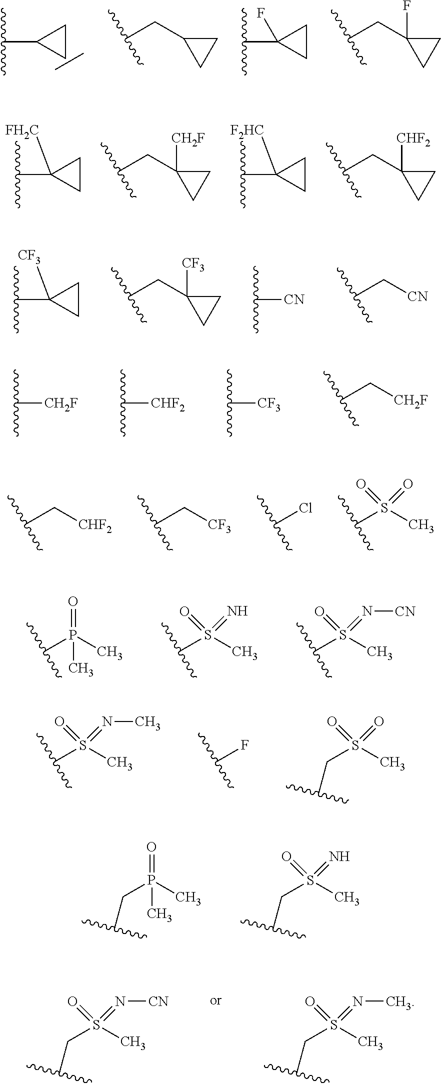

In some embodiments, R₃ is selected from the group consisting of H, D, F, —CH₃, —CD₃, —CH₂-cyclopropyl, —CH₂OH, —COOH, —CH₂CN, —CH₂F, —CHF₂, —CH₂CF₃, —C≡CH, -cyclopropyl, —CN,

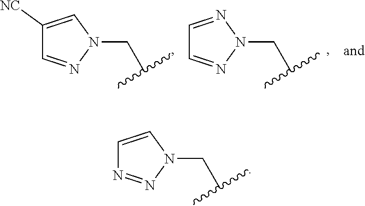 [SMILES: *Cn1cc(C#N)cn1 ; *Cn1nccn1 ; *Cn1ccnn1]

In some embodiments, R₃ is selected from the group consisting of H, F, —CH₃, —CH₂-cyclopropyl, —CH₂OH, —COOH, —CH₂CN, —CH₂F, —CHF₂, —CH₂CF₃, —C≡CH, -cyclopropyl, —CN,

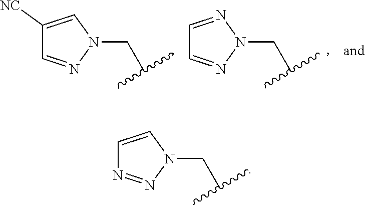 [SMILES: *Cn1cc(C#N)cn1 ; *Cn1nccn1 ; *Cn1ccnn1]

In some embodiments, R₃ is selected from the group consisting of H, D, F, —CH₃, —CD₃, —CH₂-cyclopropyl, —CH₂OH, —COOH, —CH₂CN, —CH₂F, —CHF₂, —CH₂CF₃, —C≡CH, -cyclopropyl, and —CN. In some embodiments, R₃ is H, D, F, —CD₃, or —CN.

In some embodiments, R₃ is a substituted or unsubstituted heterocyclic ring of Formulas (44)-(49):

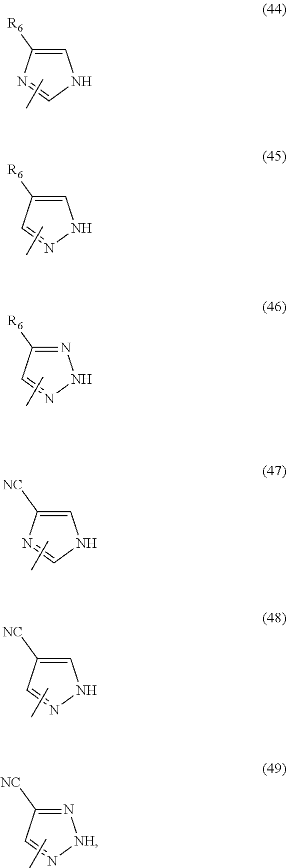

wherein R₆ is H, substituted or unsubstituted alkyl or heteroalkyl, substituted or unsubstituted alkenyl or heteroalkenyl, substituted or unsubstituted alkynyl or heteroalkynyl, substituted or unsubstituted cycloalkyl, substituted or unsubstituted cycloalkenyl, substituted or unsubstituted heterocycloalkyl, substituted or unsubstituted heterocycloalkenyl, or a combination thereof.

In some embodiments, R₃ is

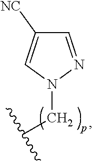

wherein p is an integer from 1 to 5. In some embodiments, p is 1. In some embodiments, p is 2. In some embodiments, p is 3. In some embodiments, p is 4. In some embodiments, p is 5.

In some embodiments, R₃ is

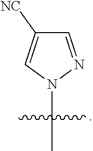 [SMILES: *n1cc(C#N)cn1]

In some embodiments, R₆ is H. In some embodiments, R₆ is D.

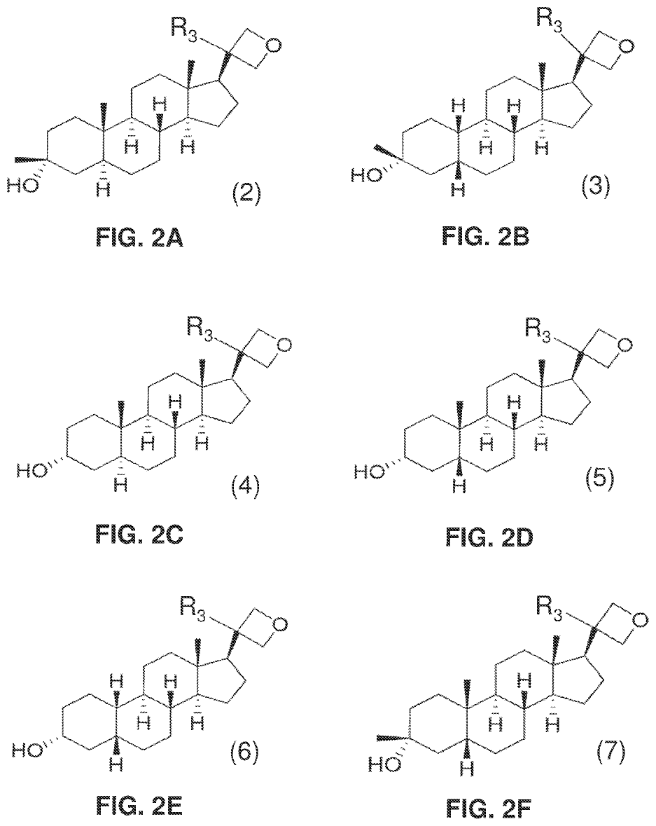

In some embodiments of formula (1), m is 0 and n is 1, 2, or 3. In some embodiments, m is 1 and n is 0, 1, 2 or 3. In some embodiments, m is 2 and n is 0, 1, 2 or 3. In some embodiments, m is 3 and n is 0, 1, 2 or 3. In some embodiments, n is 0 and m is 1, 2, or 3. In some embodiments, n is 1 and m is 0, 1, 2, or 3. In some embodiments, n is 2 and m is 0, 1, 2, or 3. In some embodiments, n is 3 and m is 0, 1, 2, or 3. In further embodiments of formula (I), m is 1 and n is 1 (e.g., as in formulas (2)-(43), (20′)-(25′) and (32′)-(37′), FIG. 2A-FIG. 8F). In some embodiments, m is 2 and n is 1. In some embodiments, m is 3 and n is 0.

In some embodiments of formula (1), R₁, R₂, R₄ and R₅ are each selected from the group consisting of H, D, —CH₃, —CD₃, —C₂H₅, —C₃H₇, —C₄H₉, —C₅H₁₁, and —C₆H₁₃; and R₃ is selected from the group consisting of H, D, F, —CH₃, —CD₃, —CH₂-cyclopropyl, —CH₂OH, —COOH, —CH₂CN, —CH₂F, —CHF₂, —CH₂CF₃, —C≡CH, -cyclopropyl, —CN,

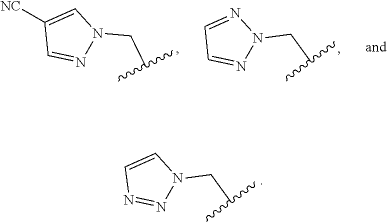 [SMILES: *Cn1cc(C#N)cn1 ; *Cn1nccn1 ; *Cn1ccnn1]

In some embodiments, R₃ is selected from the group consisting of H, D, F, —CH₃, —CD₃, and —CN.

In some embodiments of formula (1), R₁, R₂, R₄ and R₅ are each selected from the group consisting of H, D, —CH₃, —CD₃, —C₂H₅, —C₃H₇, —C₄H₉, —C₅H₁₁, and —C₆H₁₃; and R₃ is selected from the group consisting of H, F, —CH₃, —CH₂-cyclopropyl, —CH₂OH, —COOH, —CH₂CN, —CH₂F, —CHF₂, —CH₂CF₃, —C≡CH, -cyclopropyl, —CN,

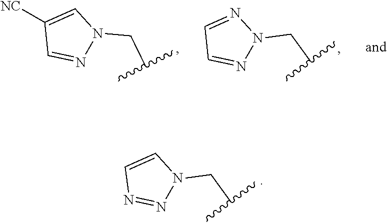 [SMILES: *Cn1cc(C#N)cn1 ; *Cn1nccn1 ; *Cn1ccnn1]

In some embodiments of formula (1), R₁, R₂, R₄ and R₅ are each selected from the group consisting of H, —CH₃, —C₂H₅, —C₃H₇, —C₄H₉, —C₅H₁₁, and —C₆H₁₃; and R₃ is selected from the group consisting of H, F, —CH₃, —CH₂-cyclopropyl, —CH₂OH, —COOH, —CH₂CN, —CH₂F, —CHF₂, —CH₂CF₃, —C≡CH, -cyclopropyl, —CN,

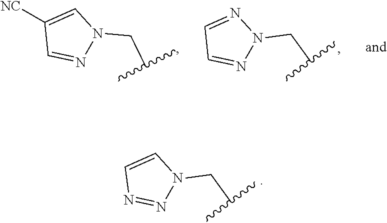 [SMILES: *Cn1cc(C#N)cn1 ; *Cn1nccn1 ; *Cn1ccnn1]

In some embodiments of formula (1), R₁, R₂, R₄ and R₅ are each selected from the group consisting of H, —CH₃, —C₂H₅, —C₃H₇, —C₄H₉, —C₅H₁₁, and —C₆H₁₃; and R₃ is selected from the group consisting of H, D, F, —CH₃, —CD₃, —CH₂-cyclopropyl, —CH₂OH, —COOH, —CH₂CN, —CH₂F, —CHF₂, —CH₂CF₃, —C≡CH, -cyclopropyl, and —CN.

In some embodiments of formula (1), R₁ and R₂ are each independently selected from the group consisting of H, —CH₃, —C₂H₅, —C₃H₇, —C₄H₉, —C₅H₁₁, and —C₆H₁₃; R₃ is selected from the group consisting of H, F, —CH₃, —CH₂-cyclopropyl, —CH₂OH, —COOH, —CH₂CN, —CH₂F, —CHF₂, —CH₂CF₃, —C≡CH, -cyclopropyl, —CN,

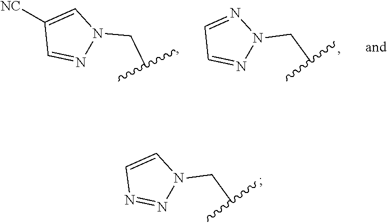 [SMILES: *Cn1cc(C#N)cn1 ; *Cn1nccn1 ; *Cn1ccnn1]

and R₄ and R₅ each is H.

In some embodiments of formula (1), R₁, R₂, R₄ and R₅ are each independently H or —CH₃; R₃ is selected from the group consisting of H, F, —CH₃, —CH₂-cyclopropyl, —CH₂OH, —COOH, —CH₂CN, —CH₂F, —CHF₂, —CH₂CF₃, —C≡CH, -cyclopropyl, —CN,

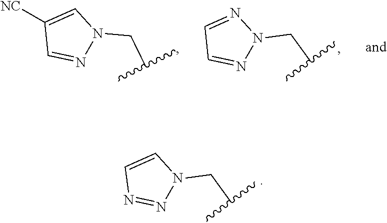 [SMILES: *Cn1cc(C#N)cn1 ; *Cn1nccn1 ; *Cn1ccnn1]

In some embodiments of formula (1), R₁ and R₂ is each independently H or —CH₃; R₃ is selected from the group consisting of H, F, —CH₃, —CH₂-cyclopropyl, —CH₂OH, —COOH, —CH₂CN, —CH₂F, —CHF₂, —CH₂CF₃, —C≡CH, -cyclopropyl, —CN,

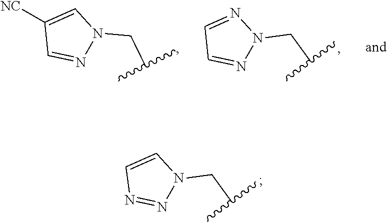 [SMILES: *Cn1cc(C#N)cn1 ; *Cn1nccn1 ; *Cn1ccnn1]

and R₄ and R₅ is H.

In some embodiments of formula (1), R₁, R₂, R₄ and R₅ each is H; and R₃ is selected from the group consisting of H, F, —CH₃, —CH₂-cyclopropyl, —CH₂OH, —COOH, —CH₂CN, —CH₂F, —CHF₂, —CH₂CF₃, —C≡CH, -cyclopropyl, —CN,

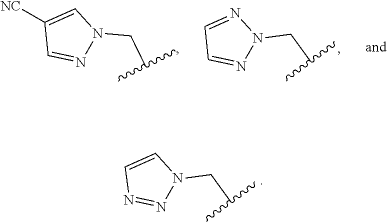 [SMILES: *Cn1cc(C#N)cn1 ; *Cn1nccn1 ; *Cn1ccnn1]

In some embodiments of formula (1), R₁, R₂, R₄ and R₅ are each selected from the group consisting of H, —CH₃, —C₂H₅, —C₃H₇, —C₄H₉, —C₅H₁₁, and —C₆H₁₃; and R₃ is selected from the group consisting of H, F, and —CN.

In some embodiments of formula (1), R₁ and R₂ are each independently selected from the group consisting of H, —CH₃, —C₂H₅, —C₃H₇, —C₄H₉, —C₅H₁₁, and —C₆H₁₃; R₃ is selected from the group consisting of R₃ is selected from the group consisting of H, F, and —CN; and R₄ and R₅ each is H.

In some embodiments of formula (1), R₁, R₂, R₄ and R₅ are each independently H or —CH₃; R₃ is selected from the group consisting of R₃ is selected from the group consisting of H, F, and —CN.

In some embodiments of formula (1), R₁ and R₂ is each independently H or —CH₃; R₃ is selected from the group consisting of R₃ is selected from the group consisting of H, F, and —CN; and R₄ and R₅ is H.

In some embodiments of formula (1), R₁, R₂, R₄ and R₅ each is H; and R₃ is selected from the group consisting of H, F, and —CN.

In some embodiments of formula (1), m is 1, n is 1; R₁, R₂, R₄ and R₅ are each selected from the group consisting of H, D, —CH₃, —CD₃, —C₂H₅, —C₃H₇, —C₄H₉, —C₅H₁₁, and —C₆H₁₃; and R₃ is selected from the group consisting of H, D, F, —CH₃, —CD₃, —CH₂-cyclopropyl, —CH₂OH, —COOH, —CH₂CN, —CH₂F, —CHF₂, —CH₂CF₃, —C≡CH, -cyclopropyl, —CN,

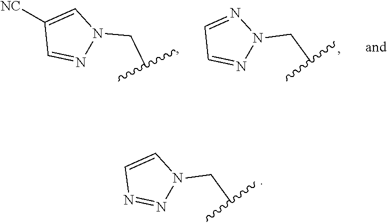 [SMILES: *Cn1cc(C#N)cn1 ; *Cn1nccn1 ; *Cn1ccnn1]

In some embodiments of formula (1), m is 1, n is 1; R₁, R₂, R₄ and R₅ are each selected from the group consisting of H, —CH₃, —C₂H₅, —C₃H₇, —C₄H₉, —C₅H₁₁, and —C₆H₁₃; and R₃ is selected from the group consisting of H, D, F, —CH₃, —CD₃, —CH₂-cyclopropyl, —CH₂OH, —COOH, —CH₂CN, —CH₂F, —CHF₂, —CH₂CF₃, —C≡CH, -cyclopropyl, —CN,

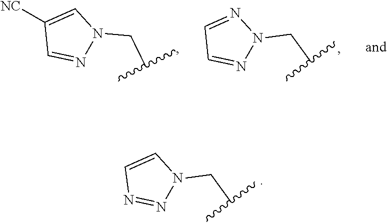 [SMILES: *Cn1cc(C#N)cn1 ; *Cn1nccn1 ; *Cn1ccnn1]

In some embodiments of formula (1), m is 1, n is 1; R₁, R₂, R₄ and R₅ are each selected from the group consisting of H, —CH₃, —C₂H₅, —C₃H₇, —C₄H₉, —C₅H₁₁, and —C₆H₁₃; and R₃ is selected from the group consisting of H, F, —CH₃, —CH₂-cyclopropyl, —CH₂OH, —COOH, —CH₂CN, —CH₂F, —CHF₂, —CH₂CF₃, —C≡CH, -cyclopropyl, —CN,

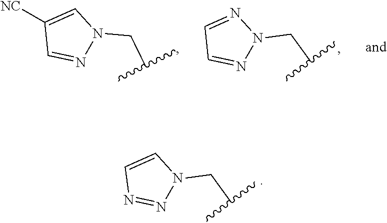 [SMILES: *Cn1cc(C#N)cn1 ; *Cn1nccn1 ; *Cn1ccnn1]

In some embodiments of formula (1), m is 1, n is 1; R₁, R₂, R₄ and R₅ are each selected from the group consisting of H, D, —CH₃, —CD₃, —C₂H₅, —C₃H₇, —C₄H₉, —C₅H₁₁, and —C₆H₁₃; and R₃ is selected from the group consisting of H, D, F, —CH₃, —CD₃, —CH₂-cyclopropyl, —CH₂OH, —COOH, —CH₂CN, —CH₂F, —CHF₂, —CH₂CF₃, —C≡CH, -cyclopropyl, and —CN.

In some embodiments of formula (1), m is 1, n is 1; R₁, R₂, R₄ and R₅ are each selected from the group consisting of H, D, —CH₃, —CD₃, —C₂H₅, —C₃H₇, —C₄H₉, —C₅H₁₁, and —C₆H₁₃; and R₃ is selected from the group consisting of H, F, —CH₃, —CH₂-cyclopropyl, —CH₂OH, —COOH, —CH₂CN, —CH₂F, —CHF₂, —CH₂CF₃, —C≡CH, -cyclopropyl, and —CN.

In some embodiments of formula (1), m is 1, n is 1; R₁, R₂, R₄ and R₅ are each selected from the group consisting of H and —CH₃; and R₃ is selected from the group consisting of H, F, —CH₃, —CH₂-cyclopropyl, —CH₂OH, —COOH, —CH₂CN, —CH₂F, —CHF₂, —CH₂CF₃, —C═CH, —C≡CH, -cyclopropyl, —CN,

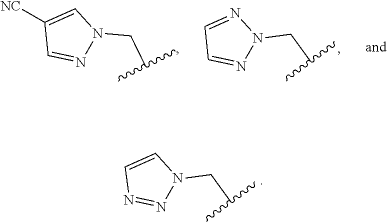 [SMILES: *Cn1cc(C#N)cn1 ; *Cn1nccn1 ; *Cn1ccnn1]

In some embodiments of formula (1), m is 1, n is 1; R₁, R₂, R₄ and R₅ each is H; and R₃ is selected from the group consisting of H, F, —CH₃, —CH₂-cyclopropyl, —CH₂OH, —COOH, —CH₂CN, —CH₂F, —CHF₂, —CH₂CF₃, —C≡CH, -cyclopropyl, —CN,

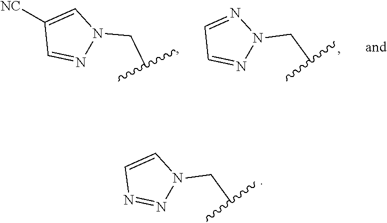 [SMILES: *Cn1cc(C#N)cn1 ; *Cn1nccn1 ; *Cn1ccnn1]

In some embodiments of formula (1), m is 1, n is 1; R₁, R₂, R₄ and R₅ are each selected from the group consisting of H and —CH₃; and R₃ is selected from the group consisting of H, F, and —CN.

In some embodiments of formula (1), m is 2, n is 1; R₁, R₂, R₄ and R₅ are each selected from the group consisting of H, —CH₃, —C₂H₅, —C₃H₇, —C₄H₉, —C₅H₁₁, and —C₆H₁₃; and R₃ is selected from the group consisting of H, F, —CH₃, —CH₂-cyclopropyl, —CH₂OH, —COOH, —CH₂CN, —CH₂F, —CHF₂, —CH₂CF₃, —C≡CH, -cyclopropyl, —CN,

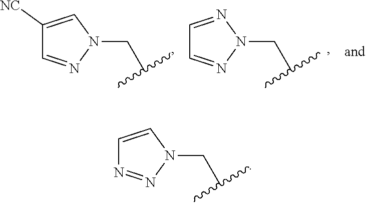 [SMILES: *Cn1cc(C#N)cn1 ; *Cn1nccn1 ; *Cn1ccnn1]

In some embodiments of formula (1), m is 2, n is 1; R₁, R₂, R₄ and R₅ are each selected from the group consisting of H and —CH₃; and R₃ is selected from the group consisting of H, F, —CH₃, —CH₂-cyclopropyl, —CH₂OH, —COOH, —CH₂CN, —CH₂F, —CHF₂, —CH₂CF₃, —C═CH, —C≡CH, -cyclopropyl, —CN,

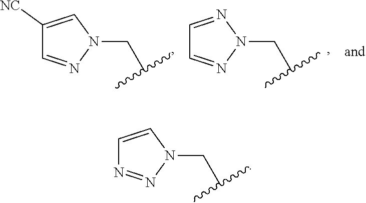 [SMILES: *Cn1cc(C#N)cn1 ; *Cn1nccn1 ; *Cn1ccnn1]

In some embodiments of formula (1), m is 2, n is 1; R₁, R₂, R₄ and R₅ each is H; and R₃ is selected from the group consisting of H, F, —CH₃, —CH₂-cyclopropyl, —CH₂OH, —COOH, —CH₂CN, —CH₂F, —CHF₂, —CH₂CF₃, —C≡CH, -cyclopropyl, —CN,

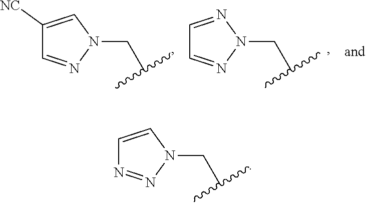 [SMILES: *Cn1cc(C#N)cn1 ; *Cn1nccn1 ; *Cn1ccnn1]

In some embodiments of formula (1), m is 3, n is 0; R₁, R₂, R₄ and R₅ are each selected from the group consisting of H, —CH₃, —C₂H₅, —C₃H₇, —C₄H₉, —C₅H₁₁, and —C₆H₁₃; and R₃ is selected from the group consisting of H, F, —CH₃, —CH₂-cyclopropyl, —CH₂OH, —COOH, —CH₂CN, —CH₂F, —CHF₂, —CH₂CF₃, —C≡CH, -cyclopropyl, —CN,

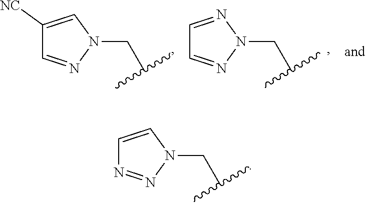 [SMILES: *Cn1cc(C#N)cn1 ; *Cn1nccn1 ; *Cn1ccnn1]

In some embodiments of formula (1), m is 3, n is 0; R₁, R₂, R₄ and R₅ are each selected from the group consisting of H, and —CH₃; and R₃ is selected from the group consisting of H, F, —CH₃, —CH₂-cyclopropyl, —CH₂OH, —COOH, —CH₂CN, —CH₂F, —CHF₂, —CH₂CF₃, —C═CH, —C≡CH, -cyclopropyl, —CN,

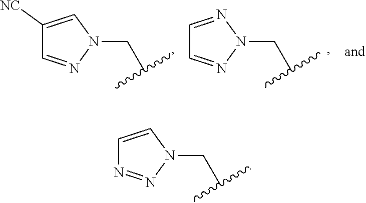 [SMILES: *Cn1cc(C#N)cn1 ; *Cn1nccn1 ; *Cn1ccnn1]

In some embodiments of formula (1), m is 3, n is 0; R₁, R₂, R₄ and R₅ each is H; and R₃ is selected from the group consisting of H, F, —CH₃, —CH₂-cyclopropyl, —CH₂OH, —COOH, —CH₂CN, —CH₂F, —CHF₂, —CH₂CF₃, —C≡CH, -cyclopropyl, —CN,

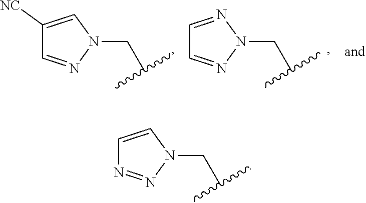 [SMILES: *Cn1cc(C#N)cn1 ; *Cn1nccn1 ; *Cn1ccnn1]

In some embodiments, the neuroactive steroid of formula (1) has a structure according to:

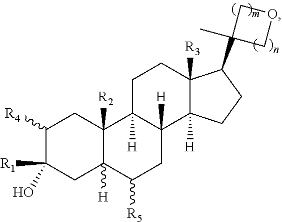

one or more isomers thereof, or a pharmaceutically acceptable salt thereof.

In some embodiments, the neuroactive steroid of formula (1) has a structure according to:

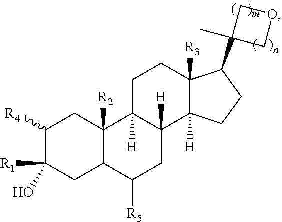

one or more isomers thereof, or a pharmaceutically acceptable salt thereof.

The neuroactive steroids suitable for this invention can comprise at least one of the R₁, R₂, R₃, R₄ and R₅ being a substituted or unsubstituted C3-C10 cycloalkyl, substituted or unsubstituted C3-C10 cycloalkenyl, substituted or unsubstituted C3-C10 heterocycloalkyl, substituted or unsubstituted C3-C10 heterocycloalkenyl, substituted or unsubstituted aryl, substituted or unsubstituted heteroaryl, or a combination thereof. At least one of the R₁, R₂, R₃, R₄ and R₅ can be a C1-C10 haloalkyl, wherein the halogen is selected from the group consisting of Cl, F, Br and I. The halogen substitutions can be on one or more carbon atoms. One carbon atom can have one or more same or different halogen substitutions.

In some embodiments, a neuroactive steroid of the present disclosure has a structure according to:

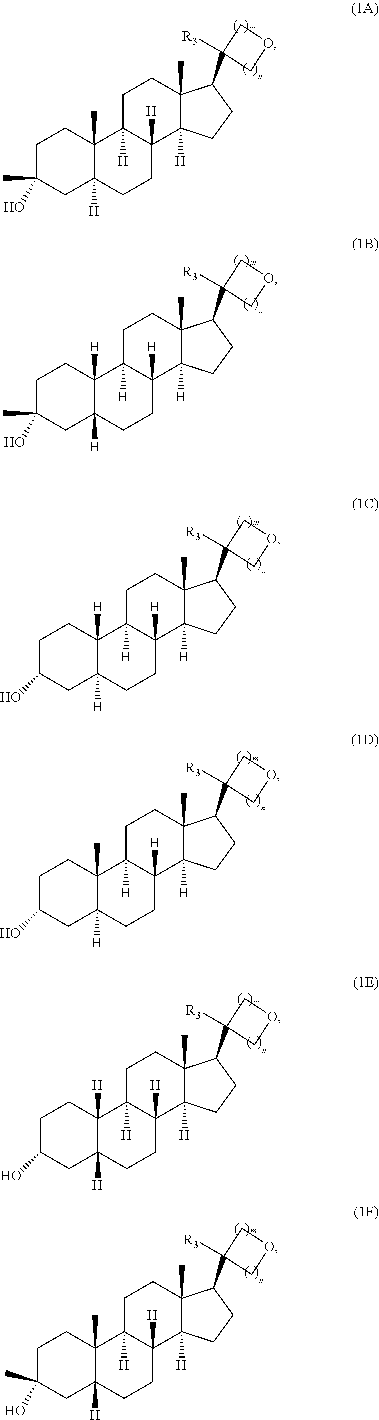

or a pharmaceutically acceptable salt thereof, wherein R₃, m, and n are as defined above in formula (1).

In some embodiments of formula (1A)-formula (1F), m is 0 and n is 1, 2, or 3. In some embodiments, m is 1 and n is 0, 1, 2 or 3. In some embodiments, m is 2 and n is 0, 1, 2 or 3. In some embodiments, m is 3 and n is 0, 1, 2 or 3. In some embodiments, n is 0 and m is 1, 2, or 3. In some embodiments, n is 1 and m is 0, 1, 2, or 3. In some embodiments, n is 2 and m is 0, 1, 2, or 3. In some embodiments, n is 3 and m is 0, 1, 2, or 3. In some embodiments, m is 1 and n is 1. In some embodiments, m is 2 and n is 1. In some embodiments, m is 3 and n is 0.

In some embodiments, a neuroactive steroid of the present disclosure has a structure according to:

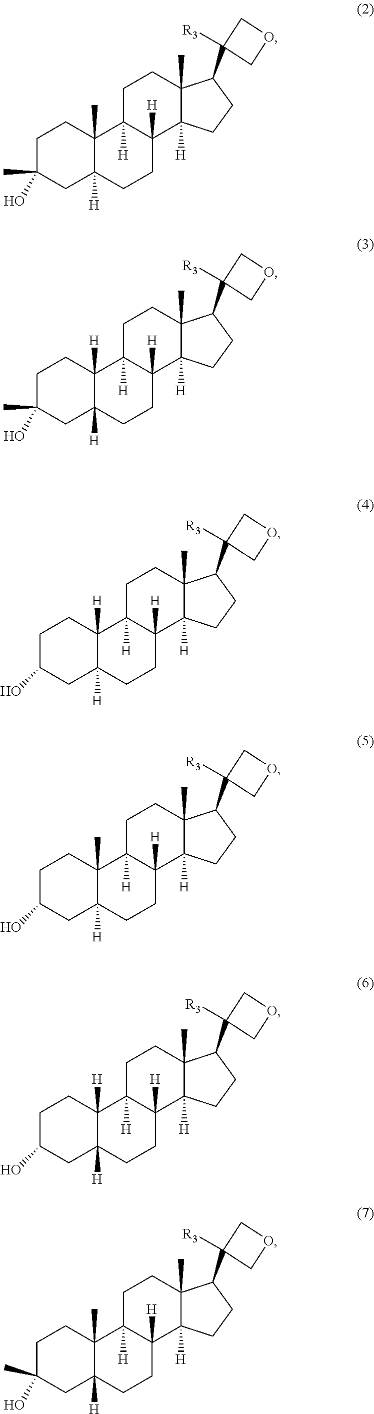

or a pharmaceutically acceptable salt thereof, wherein R₃ is as defined above in formula (1).

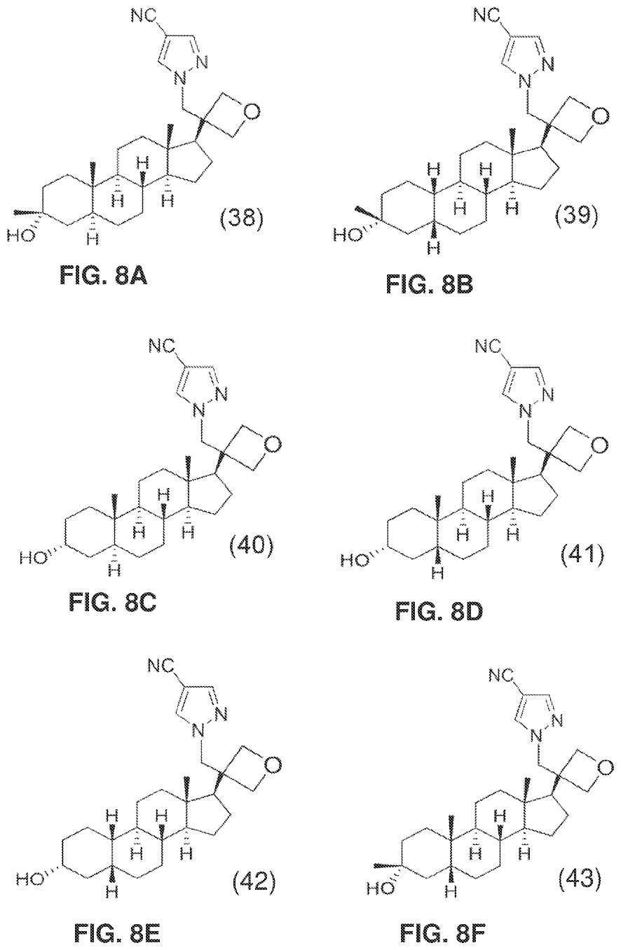

In some embodiments, the neuroactive steroid of the present disclosure is a compound of formulas (38)-(43), as shown in FIG. 8A-FIG. 8F.

In some embodiments, the neuroactive steroid of the present disclosure is a compound of formulas (2)-(43), (20′)-(25′) and (32′)-(37′), as shown in FIG. 2A-FIG. 8F.

In some embodiments, the neuroactive steroid of formula (1), is a compound of Table 1 shown below, or a pharmaceutically acceptable salt thereof.

TABLE 1
Compounds of the Present Disclosure.

| Ex# | Structure | Name |
|---|---|---|
| 1 | 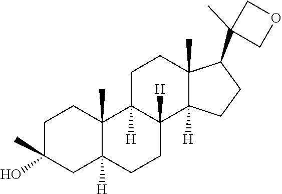 | (3R,5S,8R,9S,10S,13S,14S,17S)- 3,10,13-trimethyl-17-(3-methyloxetan-3- yl)-l,2,4,5,6,7,8,9,11,12,14,15,16,17- tetradecahydrocyclopenta[a]phenanthren- 3-ol |
| 2 | 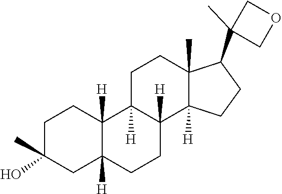 | (3R,5R,8R,9R,10S,13S,14S,17S)-3,13- dimethyl-17-(3-methyloxetan-3-yl)- 2,4,5,6,7,8,9,10,11,12,14,15,16,17- tetradecahydro-1H- cyclopenta[a]phenanthren-3-ol |
| 3 | 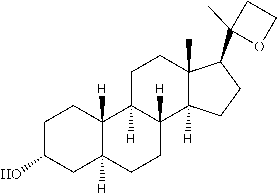 | (3R,5S,8R,9S,10S,13S,14S,17S)-10,13- dimethyl-17-(2-methyloxetan-2-yl)- 2,3,4,5,6,7,8,9,11,12,14,15,16,17- tetradecahydro-1H- cyclopenta[a]phenanthren-3-ol |
| 4 | 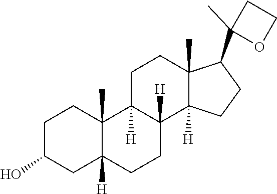 | (3R,5R,8R,9S,10S,13S,14S,17S)-10,13- dimethyl-17-(2-methyloxetan-2-yl)- 2,3,4,5,6,7,8,9,11,12,14,15,16,17- tetradecahydro-1H- cyclopenta[a]phenanthren-3-ol |
| 5 | 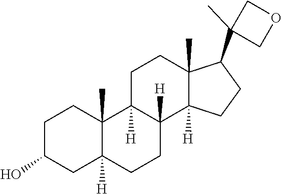 | (3R,5S,8R,9S,10S,13S,14S,17S)-10,13- dimethyl-17-(3-methyloxetan-3-yl)- 2,3,4,5,6,7,8,9,11,12,14,15,16,17- tetradecahydro-1H- cyclopenta[a]phenanthren-3-ol |
| 6 | 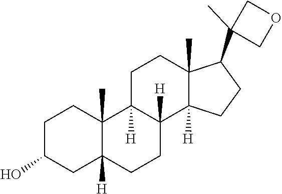 | (3R,5R,8R,9S,10S,13S,14S,17S)-10,13- dimethyl-17-(3-methyloxetan-3-yl)- 2,3,4,5,6,7,8,9,11,12,14,15,16,17- tetradecahyro-1H- cyclopenta[a]phenanthren-3-ol |
| 7 | 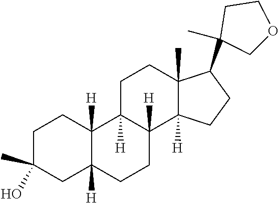 | (3R,5R,8R,9R,10S,13S,14S,17S)-3,13- dimethyl-17-(3-methyltetrahydrofuran-3- yl)-2,4,5,6,7,8,9,10,11,12,14,15,16,17- tetradecahydro-1H- cyclopenta[a]phenanthren-3-ol |
| 8 | 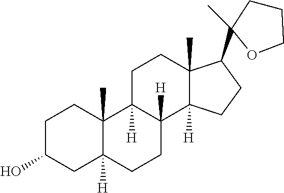 | (3R,5S,8R,9S,10S,13S,14S,17S)-10,13- dimethyl-17-(2-methyltetrahydrofuran-2- yl)-2,3,4,5,6,7,8,9,11,12,14,15,16,17- tetradecahydro-1H- cyclopenta[a]phenanthren-3-ol |
| 9 | 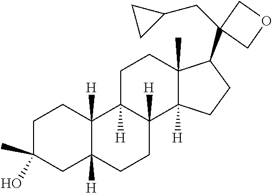 | (3R,5R,8R,9R,10S,13S,14S,17S)-17-[3- (cyclopropylmethyl)oxetan-3-yl]-3,13- dimethyl- 2,4,5,6,7,8,9,10,11,12,14,15,16,17- tetradecahydro-1H- cyclopenta[a]phenanthren-3-ol |
| 10 | 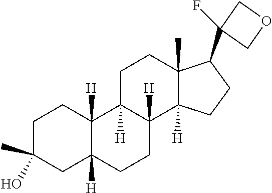 | (3R,5R,8R,9R,10S,13S,14S,17S)-17-(3- fluorooxetan-3-yl)-3,13-dimethyl- 2,4,5,6,7,8,9,10,11,12,14,15,16,17- tetradecahydro-1H- cyclopenta[a]phenanthren-3-ol |
| 11 | 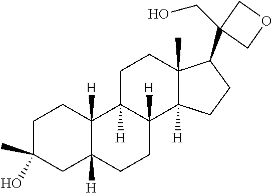 | (3R,5R,8R,9R,10S,13S,14S,17S)-17-[3- (hydroxymethyl)oxetan-3-yl]-3,13- dimethyl- 2,4,5,6,7,8,9,10,11,12,14,15,16,17- tetradecahydro-1H- cyclopenta[a]phenanthren-3-ol |
| 12 | 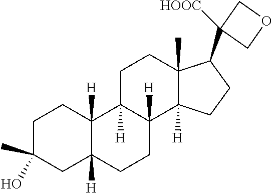 | 3-[(3R,5R,8R,9R,10S,13S,14S,17S)-3- hydroxy-3,13-dimethyl- 2,4,5,6,7,8,9,10,11,12,14,15,16,17- tetradecahydro-1H- cyclopenta[a]phenanthren-17-yl]oxetane- 3-carboxylic acid |
| 13 | 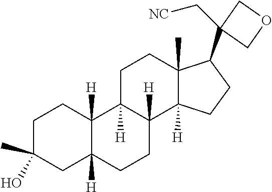 | 2-[3-[(3R,5R,8R,9R,10S,13S,14S,17S)- 3-hydroxy-3,13-dimethyl- 2,4,5,6,7,8,9,10,11,12,14,15,16,17- tetradecahydro-1H- cyclopenta[a]phenanthren-17-yl]oxetan- 3-yl]acetonitrile |
| 14 | 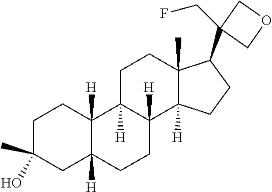 | (3R,5R,8R,9R,10S,13S,14S,17S)-17-[3- (fluoromethyl)oxetan-3-yl]-3,13- dimethyl- 2,4,5,6,7,8,9,10,11,12,14,15,16,17- tetradecahydro-1H- cyclopenta[a]phenanthren-3-ol |
| 15 | 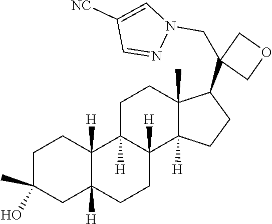 | 1-[[3-[(3R,5R,8R,9R,10S,13S,14S,17S)- 3-hydroxy-3,13-dimethyl- 2,4,5,6,7,8,9,10,11,12,14,15,16,17- tetradecahydro-1H- cyclopenta[a]phenanthren-17-yl]oxetan- 3-yl]methyl]pyrazole-4-carbonitrile |
| 16 | 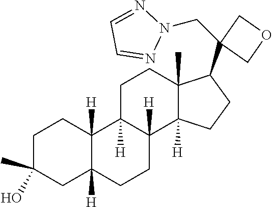 | (3R,5R,8R,9R,10S,13S,14S,17S)-3,13- dimethyl-17-[3-(triazol-2- ylmethyl)oxetan-3-yl]- 2,4,5,6,7,8,9,10,11,12,14,15,16,17- tetradecahydro-1H- cyclopenta[a]phenanthren-3-ol |
| 17 | 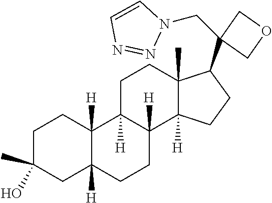 | (3R,5R,8R,9R,10S,13S,14S,17S)-3,13- dimethyl-17-[3-(triazol-1- ylmethyl)oxetan-3-yl]- 2,4,5,6,7,8,9,10,11,12,14,15,16,17- tetradecahydro-1H- cyclopenta[a]phenanthren-3-ol |
| 18 | 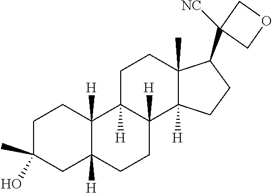 | 3-((3R,5R,8R,9R,10S,13S,14S,17S)-3- hydroxy-3,13-dimethyl- 2,4,5,6,7,8,9,10,11,12,14,15,16,17- tetradecahydro-1H- cyclopenta[a]phenanthren-17-yl)oxetane- 3-carbonitrile |
| 19 | 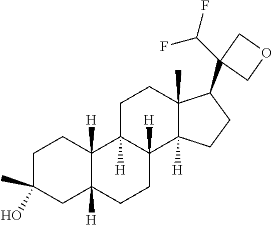 | (3R,5R,8R,9R,10S,13S,14S,17S)-17-[3- (difluoromethyl)oxetan-3-yl]-3,13- dimethyl- 2,4,5,6,7,8,9,10,11,12,14,15,16,17- tetradecahydro-1H- cyclopenta[a]phenanthren-3-ol |
| 20 | 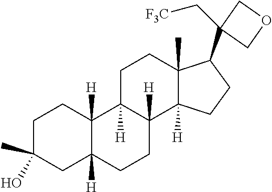 | (3R,5R,8R,9R,10S,13S,14S,17S)-3,13- dimethyl-17-(3-(2,2,2- trifluoroethyl)oxetan-3-yl)- 2,4,5,6,7,8,9,10,11,12,14,15,16,17- tetradecahydro-1H- cyclopenta[a]phenanthren-3-ol |
| 21 | 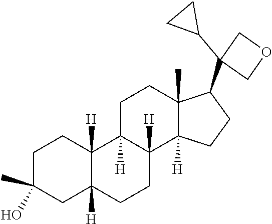 | (3R,5R,8R,9R,10S,13S,14S,17S)-17-(3- cyclopropyloxetan-3-yl)-3,13-dimethyl- 2,4,5,6,7,8,9,10,11,12,14,15,16,17- tetradecahydro-1H- cyclopenta[a]phenanthren-3-ol |
| 22 | 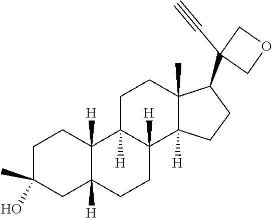 | (3R,5R,8R,9R,10S,13S,14S,17S)-17-(3- ethynyloxetan-3-yl)-3,13-dimethyl- 2,4,5,6,7,8,9,10,11,12,14,15,16,17- tetradecahydro-1H- cyclopenta[a]phenanthren-3-ol |
| 23 | 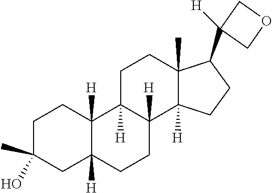 | (3R,5R,8R,9R,10S,13S,14S,17R)-17- (oxetan-3-yl)-3,13-dimethyl- 2,4,5,6,7,8,9,10,11,12,14,15,16,17- tetradecahydro-1H- cyclopenta[a]phenanthren-3-ol |
| 24 | 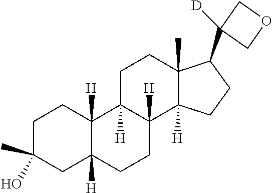 | (3R,5R,8R,9R,10S,13S,14S,17R)-3,13- dimethyl-17-(oxetan-3-yl-3-d)- 2,4,5,6,7,8,9,10,11,12,14,15,16,17- tetradecahydro-1H- cyclopenta[a]phenanthren-3-ol |
| 25 | 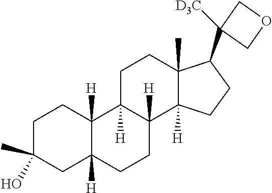 | (3R,5R,8R,9R,10S,13S,14S,17S)-3,13- dimethyl-17-(3-(methyl-d3)oxetan-3-yl)- 2,4,5,6,7,8,9,10,11,12,14,15,16,17- tetradecahydro-1H- cyclopenta[a]phenanthren-3-ol |
| 26 | 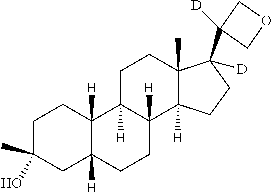 | (3R,5R,8R,9R,10S,13S,14S,17R)-3,13- dimethyl-17-(oxetan-3-yl-3-d)- 2,4,5,6,7,8,9,10,11,12,14,15,16,17- tetradecahydro-1H- cyclopenta[a]phenanthren-17-d-3-ol |
| 27 | 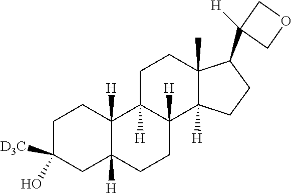 | (3R,5R,8R,9R,10S,13S,14S,17R)-13- methyl-3-(methyl-d3)-17-(oxetan-3-yl)- 2,4,5,6,7,8,9,10,11,12,14,15,16,17- tetradecahydro-1H- cyclopenta[a]phenanthren-3-ol |

The present disclosure is also directed to a pharmaceutical composition for treating a disease.

In some embodiments, the pharmaceutical composition comprises a neuroactive steroid (NAS) disclosed herein, one or more isomers thereof, a pharmaceutically acceptable salt thereof, or a combination thereof; and a pharmaceutically acceptable excipient.

In some embodiments, the pharmaceutical composition comprises a compound of formula (1), one or more isomers thereof, a pharmaceutically acceptable salt thereof, or a combination thereof; and a pharmaceutically acceptable excipient.

In some embodiments, the pharmaceutical composition of the present disclosure comprises a compound of formula (1), wherein at least one of R₁, R₂, R₃, R₄ and R₅ is a substituted or unsubstituted C3-C10 cycloalkyl, substituted or unsubstituted C3-C10 cycloalkenyl, substituted or unsubstituted C3-C10 heterocycloalkyl, substituted or unsubstituted C3-C10 heterocycloalkenyl, substituted or unsubstituted aryl, or substituted or unsubstituted heteroaryl.

In some embodiments, the pharmaceutical composition of the present disclosure comprises a compound of formula (1), wherein at least one of the R₁, R₂, R₃, R₄ and R₅ is a C1-C10 haloalkyl, wherein the halogen is one or more Cl, F, Br, I, or a combination thereof. The halogen substitutions can be on one or more carbon atoms. In some embodiments, one carbon atom has one or more same or different halogen substitutions.

In some embodiments, the pharmaceutical composition of the present disclosure comprises a compound of formula (1A), formula (1B), formula (1C), formula (1D), formula (1E), or formula (1F), or a pharmaceutically acceptable salt thereof, or a combination thereof; and a pharmaceutically acceptable excipient.

In some embodiments, the pharmaceutical composition of the present disclosure comprises a compound of formula (2)-formula (7), or a pharmaceutically acceptable salt thereof; and a pharmaceutically acceptable excipient.

In some embodiments, the pharmaceutical composition of the present disclosure comprises a compound of formulas (38)-(43), as shown in FIG. 8A-FIG. 8F; and a pharmaceutically acceptable excipient.

In some embodiments, the pharmaceutical composition of the present disclosure comprises a compound of formulas (2)-(43), (20′)-(25′) and (32′)-(37′), as shown in FIG. 2A-FIG. 8F; and a pharmaceutically acceptable excipient.

In some embodiments, the pharmaceutical composition of the present disclosure comprises a compound of Table 1; and a pharmaceutically acceptable excipient.

The pharmaceutical composition of the present disclosure can also comprise any combination of the aforementioned neuroactive steroids, such as any of those shown in formulas (1), (2)-(43), (20′)-(25′) and (32′)-(37′) and other formulas disclosed herein.

In some embodiments, the pharmaceutical composition comprises a NAS compound disclosed herein, two or more NAS compounds disclosed herein, three or more NAS compounds disclosed herein, or four or more NAS compounds disclosed herein.

In some embodiments, the pharmaceutically acceptable excipient comprises a surfactant, emulsifier, filler, carrier, isotonicifier, dispersing agent, viscosity modifier, resuspending agent, buffer or a combination thereof.

Pharmaceutical excipients typically do not have properties of a medicinal or drug active ingredient, also known as active pharmaceutical ingredient (API) and are typically used to streamline the manufacture process or packaging of the active ingredients, or to deliver an API to a patient or other subject. Pharmaceutical acceptable carrier, excipients or inactive ingredients from the Inactive Ingredients Database available from US FDA (https://www.fda.gov/drugs/drug-approvals-and-databases/inactive-ingredients-database-download) can be suitable. Some of Generally Recognized As Safe (GRAS) food substances available form US FDA's GRAS Substances (S COGS) Database (https://www.fda.gov/food/generally-recognized-safe-gras/gras-substances-scogs-database) can also be suitable.

In some embodiments, the pharmaceutically acceptable excipient is a pharmaceutically acceptable carrier. In embodiments of the present disclosure, the pharmaceutical acceptable carrier can comprise acacia, animal oils, benzyl alcohol, benzyl benzoate, calcium stearate, carbomers, cetostearyl alcohol, cetyl alcohol, cholesterol, cyclodextrins, dextrose, diethanolamine, emulsifying wax, ethylene glycol palmitostearate, glycerin, glycerin monostearate, glycerol stearate, glyceryl monooleate, glyceryl monostearate, hydrous, histidine, hydrochloric acid, hydroxypropyl cellulose, hydroxypropyl-β-cyclodextrin (HPBCD), hypromellose (hydroxypropyl methylcellulose (HPMC)), lanolin, lanolin alcohols, lecithin, medium-chain triglycerides, metallic soaps, methylcellulose, mineral oil, monobasic sodium phosphate, monoethanolamine, oleic acid, polyethylene glycols (PEG 3350, PEG 4000, PEG 6000), polyoxyethylene-polyoxypropylene copolymer (poloxamer), polyoxyethylene alkyl ethers, polyoxyethylene castor oil, polyoxyethylene castor oil derivatives, polyoxyethylene sorbitan fatty acid esters, polyoxyethylene stearates, polysorbate, polyoxyethylene (20) sorbitan monolaurate (Tween 20, Polysorbate 20), polyoxyethylene (20) sorbitan monooleate (Tween 80, Polysorbate 80), povidone, propylene glycol alginate, saline, sodium chloride, sodium citrate, sodium citrate dihydrate, sodium hydroxide, sodium lauryl sulfate, sodium phosphate monobasic, sodium phosphate dibasic, sorbitan esters, stearic acid, stearyl alcohol, sunflower oil, tragacanth, triethanolamine, vegetable oils, water, xanthan gum, or a combinations thereof.

In some embodiments, the pharmaceutical acceptable carrier comprises dextrose, glycerin, histidine, hydrochloric acid, hydroxypropyl cellulose, hydroxypropyl-β-cyclodextrin (HPBCD), hypromellose (hydroxypropyl methylcellulose (HPMC)), polyoxyethylene (20) sorbitan monolaurate (Tween 20, Polysorbate 20), polyethylene glycols (PEG 3350, PEG 4000, PEG 6000), polyoxyethylene-polyoxypropylene copolymer (Poloxamer 188, Poloxamer 407), polyoxyethylene (20) sorbitan monooleate (Tween 80, Polysorbate 80), saline, sodium chloride, sodium citrate, sodium citrate dihydrate, sodium lauryl sulfate, sodium phosphate monobasic, sodium phosphate dibasic, or a combination thereof.

The present disclosure is further directed to a method for treating a disease or condition in a subject in need thereof, the method comprising administering to the subject a therapeutically effective dosage of a compound or pharmaceutical composition disclosed herein. Any of the compounds and pharmaceutical compositions disclosed herein or a combination thereof can be suitable for treatment of the disease or condition.

Exemplary CNS diseases and conditions related to GABA-modulation include, but are not limited to, sleep disorders (e.g., insomnia), mood disorders (e.g., depression, dysthymic disorder (e.g., mild depression), bipolar disorder (e.g., I and/or II), anxiety disorders (e.g., generalized anxiety disorder (GAD), social anxiety disorder), stress, post-traumatic stress disorder (PTSD), compulsive disorders (e.g., obsessive compulsive disorder (OCD)), schizophrenia spectrum disorders (e.g., schizophrenia, schizoaffective disorder), convulsive disorders (e.g., epilepsy (e.g., status epilepticus (SE)), seizures), disorders of memory and/or cognition (e.g., attention disorders (e.g., attention deficit hyperactivity disorder (ADHD)), dementia (e.g., Alzheimer's type dementia, Lewis body type dementia, vascular type dementia), movement disorders (e.g., Huntington's disease, Parkinson's disease), personality disorders (e.g., anti-social personality disorder, obsessive compulsive personality disorder), autism spectrum disorders (ASD) (e.g., autism, monogenetic causes of autism such as synaptophathy's, e.g., Rett syndrome, Fragile X syndrome, Angelman syndrome), pain (e.g., neuropathic pain, injury related pain syndromes, acute pain, chronic pain), traumatic brain injury (TBI), vascular diseases (e.g., stroke, ischemia, vascular malformations), substance abuse disorders and/or withdrawal syndromes (e.g., addition to opiates, cocaine, and/or alcohol), tinnitus or a combination thereof. In some embodiments, CDD, MDD, PPD, essential tremor, PTSD, SE, ESE, Fragile X syndrome, Parkinson's Disease, treatment resistant depression. In some embodiments, the CNS disease or condition is CDD, MDD, PPD, excessive tremor, PTSD, SE, ESE, or Fragile X syndrome.

In some embodiments of the method disclosed herein, the disease or condition comprises sleep disorders, insomnia, mood disorders, depression, dysthymic disorder, mild depression, bipolar disorder, anxiety disorders, generalized anxiety disorder (GAD), social anxiety disorder, stress, post-traumatic stress disorder (PTSD), compulsive disorders, obsessive compulsive disorder (OCD), schizophrenia spectrum disorders, schizophrenia, schizoaffective disorder, convulsive disorders, epilepsy, status epilepticus (SE), seizures, disorders of memory and/or cognition, attention disorders, attention deficit hyperactivity disorder (ADHD), dementia, Alzheimer's type dementia, Lewis body type dementia, vascular type dementia, movement disorders, Huntington's disease, Parkinson's disease, personality disorders, anti-social personality disorder, obsessive compulsive personality disorder, autism spectrum disorders (ASD), autism, monogenetic causes of autism, synaptophathy's, Rett syndrome, Fragile X syndrome, Angelman syndrome, neuropathic pain, injury related pain syndromes, acute pain, chronic pain, traumatic brain injury (TBI), vascular diseases, stroke, ischemia, vascular malformations, substance abuse disorders and/or withdrawal syndromes, addition to opiates, addition to cocaine, addition to alcohol, tinnitus, or a combination thereof.

In some embodiments of the present method, the disease is anxiety, massive depression disorder, postpartum disorder, Alzheimer disease, Parkinson disease, epilepsy, focal onset seizures, PCDH19 pediatric epilepsy, pediatric genetic epilepsies, CDKL5 Deficiency Disorder (CDD), catamenial epilepsy, infantile spasms, Fragile X syndrome, depression, postpartum depression or premenstrual syndrome.

In some embodiments, the present disclosure is directed to the use of a neuroactive steroid disclosed herein for manufacturing a medicament for treating a disease, wherein the disease comprises sleep disorders, insomnia, mood disorders, depression, dysthymic disorder, mild depression, bipolar disorder, anxiety disorders, generalized anxiety disorder (GAD), social anxiety disorder, stress, post-traumatic stress disorder (PTSD), compulsive disorders, obsessive compulsive disorder (OCD), schizophrenia spectrum disorders, schizophrenia, schizoaffective disorder, convulsive disorders, epilepsy, status epilepticus (SE), seizures, disorders of memory and/or cognition, attention disorders, attention deficit hyperactivity disorder (ADHD), dementia, Alzheimer's type dementia, Lewis body type dementia, vascular type dementia, movement disorders, Huntington's disease, Parkinson's disease, personality disorders, anti-social personality disorder, obsessive compulsive personality disorder, autism spectrum disorders (ASD), autism, monogenetic causes of autism, synaptophathy's, Rett syndrome, Fragile X syndrome, Angelman syndrome, neuropathic pain, injury related pain syndromes, acute pain, chronic pain, traumatic brain injury (TBI), vascular diseases, stroke, ischemia, vascular malformations, substance abuse disorders and/or withdrawal syndromes, addition to opiates, addition to cocaine, addition to alcohol, tinnitus, or a combination thereof.

Any of the neuroactive steroids disclosed herein or a combination thereof can be suitable for treating the aforementioned diseases and conditions.

The pharmaceutical composition can be administered to the subject via intramuscular (IM) injection, subcutaneous (SC) injection, intravenous (IV) injection, oral administration, topical application, implant application or a combination thereof.

In some embodiments, the NAS compounds disclosed herein have superior medicinal properties.

The present disclosure now will be exemplified in the following non-limiting examples.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Example 1: (3R,5S,8R,9S,10S,13S,14S,17S)-3,10,13-trimethyl-17-(3-methyloxetan-3-yl)-1,2,4,5,6,7,8,9,11,12,14,15,16,17-tetradecahydrocyclopenta[a]phenanthren-3-ol

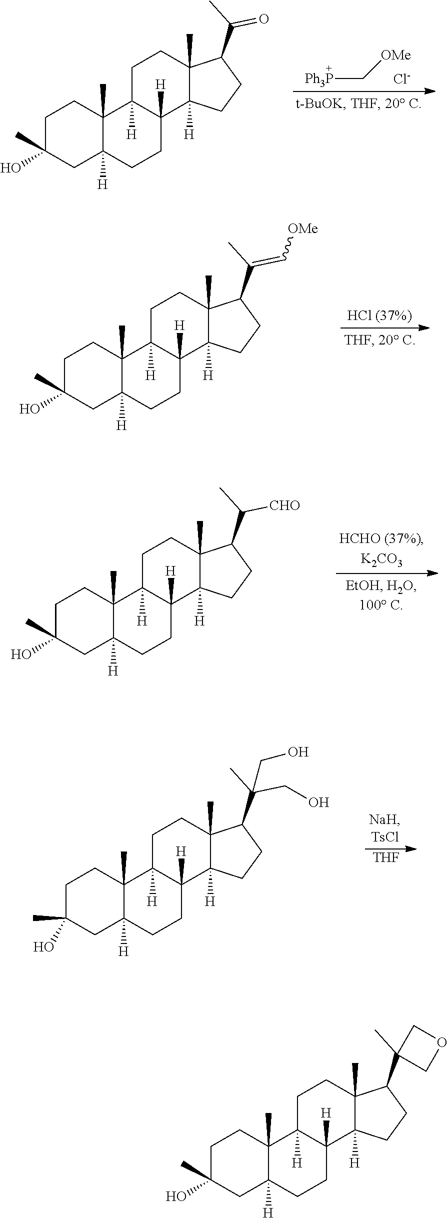

### Preparation of (3R,5S,8R,9S,10S,13S,14S,17S)-17-(2-methoxy-1-methyl-vinyl)-3,10,13-trimethyl-1,2,4,5,6,7,8,9,11,12,14,15,16,17-tetradecahydrocyclopenta[a]phenanthren-3-ol

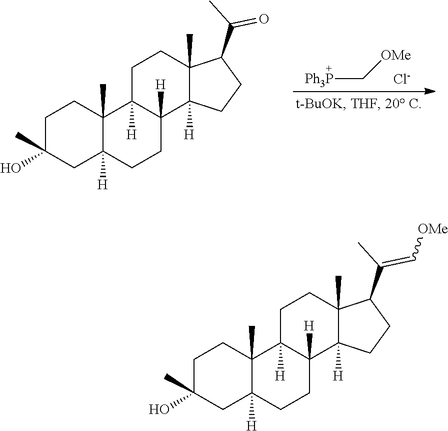

To a solution of 1-[(3R,5S,8R,9S,10S,13S,14S,17S)-3-hydroxy-3,10,13-trimethyl-1,2,4,5,6,7,8,9,11,12,14,15,16,17-tetradecahydrocyclopenta[a]phenanthren-17-yl]ethanone (1.00 g, 3.01 mmol, 1 eq) and methoxymethyl(triphenyl)phosphonium chloride (1.34 g, 3.91 mmol, 1.3 eq) in THF (10 mL) was added potassium 2-methylpropan-2-olate (439 mg, 3.91 mmol, 1.3 eq). The mixture was stirred at 20° C. for 16 h. The reaction mixture was quenched with H₂O (10 mL) and extracted with DCM (10 mL×3). The combined organic layers were washed with H₂O (20 mL), dried over Na₂SO₄, filtered, and concentrated under reduced pressure. The residue was purified by flash silica gel chromatography (ISCO® 12 g SepaFlash® Silica Flash Column, eluted with 0-30% ethyl acetate/petroleum ether gradient @ 25 mL/min) to produce (3R,5S,8R,9S,10S,13S,14S,17S)-17-(2-methoxy-1-methyl-vinyl)-3,10,13-trimethyl-1,2,4,5,6,7,8,9,11,12,14,15,16,17-tetradecahydrocyclopenta[a]phenanthren-3-ol (950 mg, 87.6% yield) as a white solid, which is a mixture of E and Z isomers with a ratio of ˜3:1. Major isomer ¹H NMR (400 MHz, CDCl₃) δ (ppm) 5.79 (s, 1H), 3.57 (s, 3H), 1.95-0.85 (m, 28H), 0.82-0.70 (m, 4H), 0.56 (s, 3H).

### Preparation of 2-[(3R,5S,8R,9S,10S,13S,14S,17R)-3-hydroxy-3,10,13-trimethyl-1,2,4,5,6,7,8,9,11,12,14,15,16,17-tetradecahydrocyclopenta[a]phenanthren-17-yl]propanal

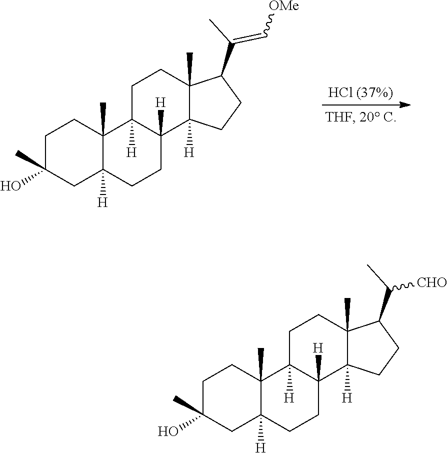

To a solution of (3R,5S,8R,9S,10S,13S,14S,17S)-17-(2-methoxy-1-methyl-vinyl)-3,10,13-trimethyl-1,2,4,5,6,7,8,9,11,12,14,15,16,17-tetradecahydrocyclopenta[a]phenanthren-3-ol (950 mg, 2.63 mmol, 1 eq) in THF (5 mL) was added HCl (510 mg, 5.18 mmol, 0.5 mL, 37% in H₂O, 1.96 eq). The mixture was stirred at 20° C. for 0.5 h. The reaction mixture was neutralized by saturated Na₂CO₃ to pH˜7, and then extracted with DCM (10 mL×3). The combined organic layers were washed with H₂O (10 mL), dried over Na₂SO₄, filtered and concentrated under reduced pressure. The residue was purified by flash silica gel chromatography (ISCO®; 4 g SepaFlash® Silica Flash Column, eluted with 0-20% ethyl acetate/petroleum ether gradient @ 25 mL/min) to give 2-[(3R,5S,8R,9S,10S,13S,14S,17R)-3-hydroxy-3,10,13-trimethyl-1,2,4,5,6,7,8,9,11,12,14,15,16,17-tetradecahydrocyclopenta[a]phenanthren-17-yl]propanal (900 mg, 98.6% yield) as a white solid. ¹H NMR (400 MHz, CDCl₃) δ (ppm) 9.53 (d, J=5.2 Hz, 1H), 2.42-2.24 (m, 1H), 1.96-1.78 (m, 1H), 1.70-0.76 (m, 27H), 0.86-0.70 (m, 4H), 0.67 (s, 3H).

### Preparation of 2-[(3R,5S,8R,9S,10S,13S,14S,17S)-3-hydroxy-3,10,13-trimethyl-1,2,4,5,6,7,8,9,11,12,14,15,16,17-tetradecahydrocyclopenta[a]phenanthren-17-yl]-2-methyl-propane-1,3-diol

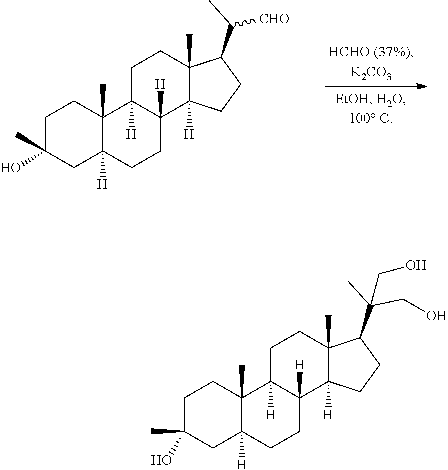

A mixture of 2-[(3R,5S,8R,9S,10S,13S,14S,17R)-3-hydroxy-3,10,13-trimethyl-1,2,4,5,6,7,8,9,11,12,14,15,16,17-tetradecahydrocyclopenta[a]phenanthren-17-yl]propanal (400 mg, 1.15 mmol, 1 eq), HCHO (8.72 g, 107.45 mmol, 8.00 mL, 37%, 93.09 eq), K₂CO₃ (638.10 mg, 4.62 mmol, 4 eq) in H₂O (5 mL) and EtOH (5 mL) was degassed and purged with N₂ for 3 times, and then the mixture was stirred at 100° C. for 16 h under N₂ atmosphere. The suspension was filtered and the resulting residue was washed with H₂O (10 mL) to give crude product of 2-[(3R,5S,8R,9S,10S,13S,14S,17S)-3-hydroxy-3,10,13-trimethyl-1,2,4,5,6,7,8,9,11,12,14,15,16,17-tetradecahydrocyclopenta[a]phenanthren-17-yl]-2-methyl-propane-1,3-diol (390 mg, 89.3% yield) as a white solid. ¹H NMR (400 MHz, CD₃OD) δ (ppm) 3.68-3.51 (m, 2H), 3.37 (m, 2H), 2.02-1.89 (m, 1H), 1.76-0.67 (m, 34H).

### Preparation of (3R,5S,8R,9S,10S,13S,14S,17S)-3,10,13-trimethyl-17-(3-methyloxetan-3-yl)-1,2,4,5,6,7,8,9,11,12,14,15,16,17-tetradecahydrocyclopenta[a]phenanthren-3-ol

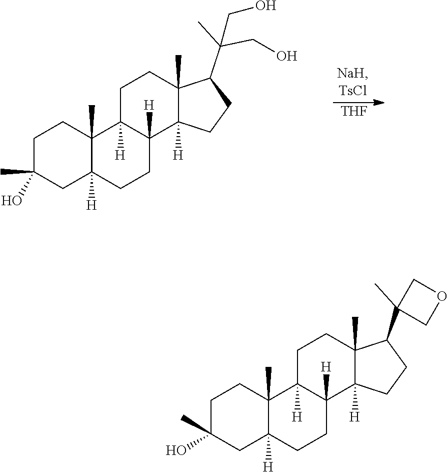

To a solution of 2-[(3R,5S,8R,9S,10S,13S,14S,17S)-3-hydroxy-3,10,13-trimethyl-1,2,4,5,6,7,8,9,11,12,14,15,16,17-tetradecahydrocyclopenta[a]phenanthren-17-yl]-2-methyl-propane-1,3-diol (200 mg, 0.528 mmol, 1 eq) in THF (3 mL) was added NaH (25.4 mg, 0.634 mmol, 60% in mineral oil, 1.2 eq), the mixture was stirred at 20° C. for 0.5 h. Then p-toluenesulfonyl chloride (101 mg, 0.528 mmol, 1 eq) was added, and the mixture was stirred at 20° C. for 1 h before another portion of NaH (25.4 mg, 0.634 mmol, 60%, 1.2 eq) was added. The resulting mixture was stirred at 20° C. for an additional 16 h. The mixture was then quenched by water (3 mL) and extracted with EtOAc (5 mL×3). The combined organic layer was dried over Na₂SO₄, filtered and concentrated. The residue was purified by flash silica gel chromatography (ISCO®; 4 g SepaFlash® Silica Flash Column, eluted with 0-40% ethyl acetate/petroleum ether gradient @ 20 mL/min) to give product (60 mg, 31% yield). The product was then recrystallized from EtOAc (2 mL) to obtain (3R,5S,8R,9S,10S,13S,14S,17S)-3,10,13-trimethyl-17-(3-methyloxetan-3-yl)-1,2,4,5,6,7,8,9,11,12,14,15,16,17-tetradecahydrocyclopenta[a]phenanthren-3-ol (10 mg, 5.17% yield, 98.5% purity) as colorless crystals. LCMS (ESI) m/z, C₂₄H₄₀O₂: calculated 360.3, found [M-OH]⁺: 343.3. ¹H NMR (400 MHz, CDCl₃) δ (ppm) 4.85 (d, J=6.0 Hz, 1H), 4.59 (d, J=5.2 Hz, 1H), 4.20 (d, J=6.0 Hz, 1H), 4.14 (d, J=5.2 Hz, 1H), 2.09-1.79 (m, 3H), 1.76-1.63 (m, 2H), 1.61-0.82 (m, 23H), 0.80-0.66 (m, 4H), 0.53 (s, 3H). ¹³C NMR (100 MHz, CDCl₃) δ (ppm) 83.55, 79.93, 69.78, 56.38, 55.26, 54.04, 43.46, 41.93, 41.77, 41.08, 39.86, 35.51, 35.03, 34.86, 31.91, 28.38, 26.36, 24.30, 24.24, 20.73, 12.38, 11.19.

### Example 2: (3R,5R,8R,9R,10S,13S,14S,17S)-3,13-dimethyl-17-[3-methyloxetan-3-yl)-2,4,5,6,7,8,9,10,11,12,14,15,16,17-tetradecahydro-1H-cyclopenta[a]phenanthren-3-ol

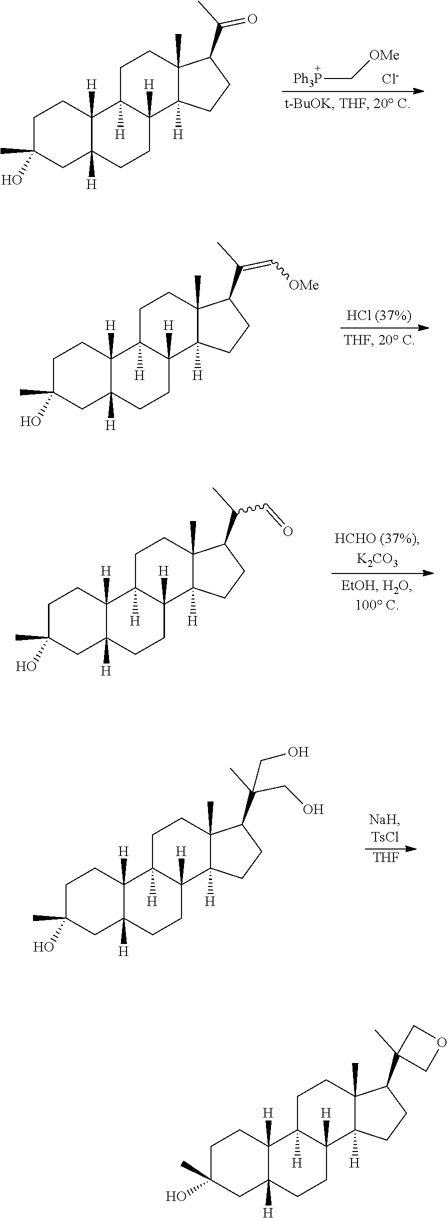

Compound (3R,5R,8R,9R,10S,13S,14S,17S)-3,13-dimethyl-17-(3-methyloxetan-3-yl)-2,4,5,6,7,8,9,10,11,12,14,15,16,17-tetradecahydro-1H-cyclopenta[a]phenanthren-3-ol was prepared using the same reaction sequences as the preparation of (3R,5S,8R,9S,10S,13S,14S,17S)-3,10,13-trimethyl-17-(3-methyloxetan-3-yl)-1,2,4,5,6,7,8,9,11,12,14,15,16,17-tetradecahydrocyclopenta[a]phenanthren-3-ol, except replacing 1-[(3R,5S,8R,9S,10S,13S,14S,17S)-3-hydroxy-3,10,13-trimethyl-1,2,4,5,6,7,8,9,11,12,14,15,16,17-tetradecahydrocyclopenta[a]phenanthren-17-yl]ethanone with 1-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]ethanone (23 mg, 23.7% yield, 98% purity, white solid). LCMS (ESI) m/z, C₂₃H₃₈O₂: calculated 346.3, found [M-OH]⁺: 329.3. ¹H NMR (400 MHz, CDCl₃) 4.85 (d, J=6.0 Hz, 1H), 4.59 (d, J=5.2 Hz, 1H), 4.21 (d, J=6.0 Hz, 1H), 4.14 (d, J=5.2 Hz, 1H), 2.05-0.86 (m, 30H), 0.54 (s, 3H). ¹³C NMR (100 MHz, CDCl₃) δ (ppm) 83.58, 79.85, 72.07, 55.40, 43.61, 41.93, 41.20, 39.90, 37.55, 34.71, 34.50, 31.40, 26.45, 25.99, 25.55, 25.38, 24.39, 24.13, 12.35.

### Example 3: (3R,5S,8R,9S,10S,13S,14S,17S)-10,13-dimethyl-17-(2-methyloxetan-2-yl)-2,3,4,5,6,7,8,9,11,12,14,15,16,17-tetradecahydro-1H-cyclopenta[a]phenanthren-3-ol

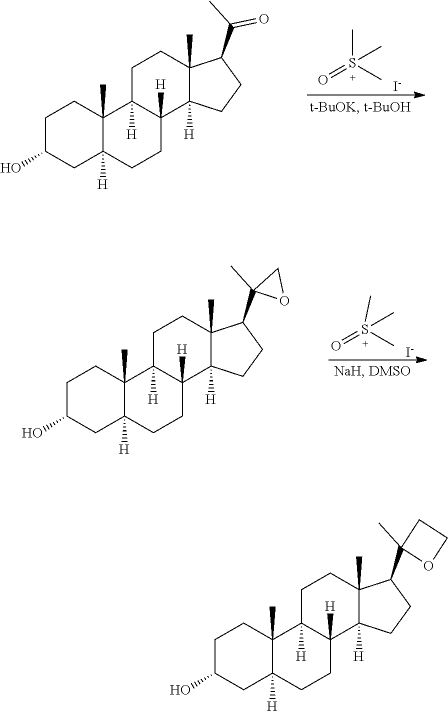

### Preparation of (3R,5S,8R,9S,10S,13S,14S,17S)-10,13-dimethyl-17-(2-methyloxiran-2-yl)-2,3,4,5,6,7,8,9,11,12,14,15,16,17-tetradecahydro-1H-cyclopenta[a]phenanthren-3-ol

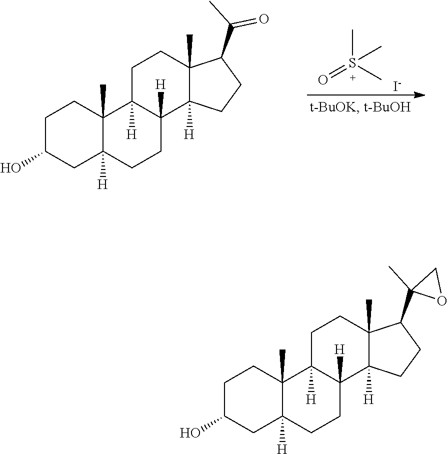

A mixture of potassium tert-butoxide (2.11 g, 18.8 mmol, 3 eq) and trimethylsulfoxonium iodide (4.15 g, 18.8 mmol, 3 eq) in t-BuOH (25 mL) was stirred at 70° C. for 1 h before 1-[(3R,5S,8R,9S,10S,13S,14S,17S)-3-hydroxy-10,13-dimethyl-2,3,4,5,6,7,8,9,11,12,14,15,16,17-tetradecahydro-1H-cyclopenta[a]phenanthren-17-yl]ethanone (2.00 g, 6.28 mmol, 1 eq) was added. The resulting mixture was stirred at 70° C. for another 50 h, then concentrated. The residue was purified by flash silica gel chromatography (ISCO®; 20 g SepaFlash® Silica Flash Column, eluted with 0-10% ethyl acetate/petroleum ether gradient @ 20 mL/min) to give (3R,5S,8R,9S,10S,13S, 14S,17S)-10,13-dimethyl-17-(2-methyloxiran-2-yl)-2,3,4,5,6,7,8,9,11,12,14,15,16,17-tetradecahydro-1H-cyclopenta[a]phenanthren-3-ol (900 mg, 43% yield) as white solid.

### Preparation of (3R,5S,8R,9S,10S,13S,14S,17S)-10,13-dimethyl-17-(2-methyloxetan-2-yl)-2,3,4,5,6,7,8,9,11,12,14,15,16,17-tetradecahydro-1H-cyclopenta[a]phenanthren-3-ol

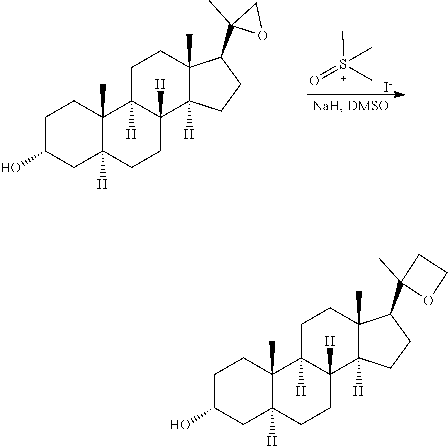

To a solution of trimethylsulfoxonium iodide (596 mg, 2.71 mmol, 3 eq) in DMSO (5 mL) was added NaH (180 mg, 4.51 mmol, 60% in mineral oil, 5 eq). The mixture was stirred at room temperature for 1 h before (3R,5S,8R,9S,10S,13S,14S,17S)-10,13-dimethyl-17-(2-methyloxiran-2-yl)-2,3,4,5,6,7,8,9,11,12,14,15,16,17-tetradecahydro-1H-cyclopenta[a]phenanthren-3-ol (299 mg, 0.90 mmol, 1 eq) was added, and the resulting mixture was stirred for another 16 h. The mixture was diluted with water (15 mL) and extracted with EtOAc (15 mL×2). The organic layers were combined, washed with brine (20 mL), dried (Na₂SO₄), filtered and concentrated under reduced pressure. The resulting residue was purified by flash silica gel chromatography (ISCO®; 4 g SepaFlash® Silica Flash Column, eluted with 0-10% ethyl acetate/petroleum ether gradient @ 15 mL/min). The product (combined with another batch) was purified again by prep-TLC (dichloromethane/ethyl acetate=30/1) to give (3R,5S,8R,9S,10S,13S,14S,17S)-10,13-dimethyl-17-(2-methyloxetan-2-yl)-2,3,4,5,6,7,8,9,11,12,14,15,16,17-tetradecahydro-1H-cyclopenta[a]phenanthren-3-ol (19.8 mg, 3.1% yield) as a white solid. LCMS (ESI) m/z, C₂₃H₃₈O₂: calculated 346.29, found [M-OH]⁺: 329.28. ¹H NMR (400 MHz, CDCl₃) δ (ppm) 4.54-4.50 (m, 1H) 4.37-4.35 (m, 1H), 4.05 (s, 1H), 2.63-2.56 (m, 1H), 2.20-2.11 (m, 2H), 2.04-2.01 (m, 1H), 1.89-1.88 (m, 1H), 1.69-1.67 (m, 4H), 1.57-0.88 (m, 19H), 0.78-0.70 (m, 7H). ¹³C NMR (100 MHz, CDCl₃) δ (ppm) 89.02, 66.58, 64.59, 59.42, 56.81, 54.22, 43.00, 39.96, 39.11, 36.06, 35.85, 34.90, 33.41, 32.15, 31.89, 28.99, 28.52, 28.32, 23.85, 22.85, 20.50, 12.65, 11.17.

### Example 4: (3R,5R,8R,9S,10S,13S,14S,17S)-10,13-dimethyl-17-(2-methyloxetan-2-yl)-2,3,4,5,6,7,8,9,11,12,14,15,16,17-tetradecahydro-1H-cyclopenta[a]phenanthren-3-ol

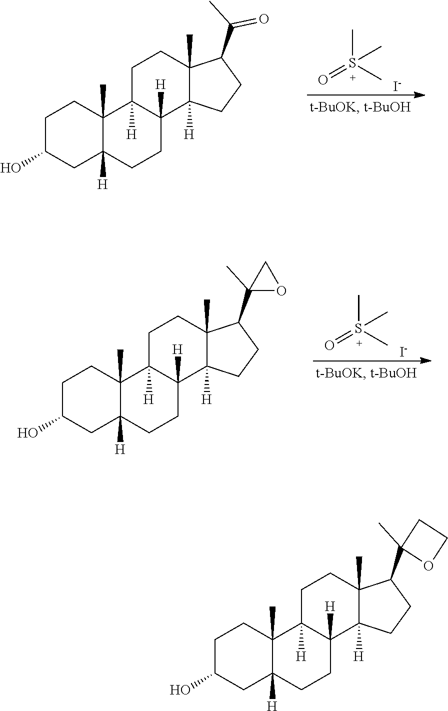

### Preparation of (3R,5R,8R,9S,10S,13S,14S,17S)-10,13-dimethyl-17-(2-methyloxiran-2-yl)-2,3,4,5,6,7,8,9,11,12,14,15,16,17-tetradecahydro-1H-cyclopenta[a]phenanthren-3-ol

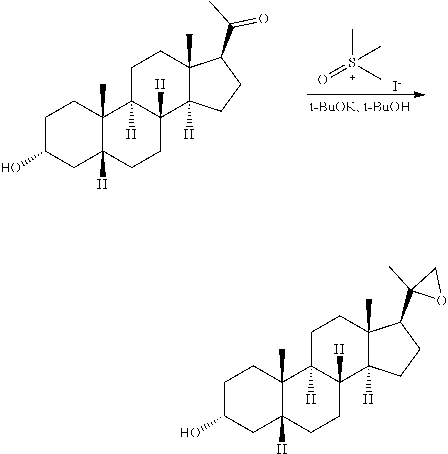

To a solution of potassium tert-butoxide (2.11 g, 18.8 mmol, 3 eq) in t-BuOH (25 mL) was added trimethylsulfoxonium iodide (4.15 g, 18.8 mmol, 3 eq). The mixture was stirred at 40° C. for 1 h before 1-[(3R,5R,8R,9S,10S,13S,14S,17S)-3-hydroxy-10,13-dimethyl-2,3,4,5,6,7,8,9,11,12,14,15,16,17-tetradecahydro-1H-cyclopenta[a]phenanthren-17-yl]ethanone (2.00 g, 6.28 mmol, 1 eq) was added, and the resulting mixture was stirred at 40° C. for 40 h. The mixture was diluted with water (30 mL), extracted with EtOAc (30 mL×3). The combined organic layer was dried over Na₂SO₄, filtered and concentrated. The residue was purified by flash silica gel chromatography (ISCO®; 40 g SepaFlash® Silica Flash Column, eluent of 0-35% ethyl acetate/petroleum ether gradient @ 30 mL/min) to give (3R,5R,8R,9S,10S,13S,14S,17S)-10,13-dimethyl-17-(2-methyloxiran-2-yl)-2,3,4,5,6,7,8,9,11,12,14,15,16,17-tetradecahydro-1H-cyclopenta[a]phenanthren-3-ol (750 mg, 36% yield) as a white solid.

### Preparation of (3R,5R,8R,9S,10S,13S,14S,17S)-10,13-dimethyl-17-(2-methyloxetan-2-yl)-2,3,4,5,6,7,8,9,11,12,14,15,16,17-tetradecahydro-1H-cyclopenta[a]phenanthren-3-ol

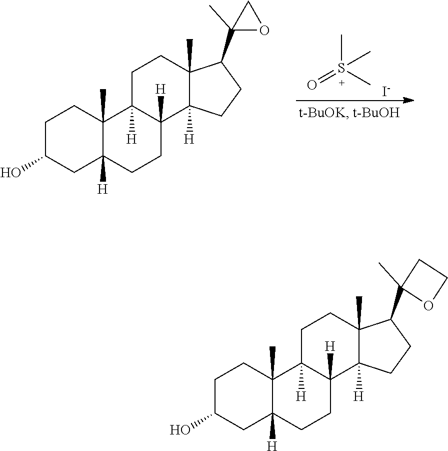

To a solution of potassium tert-butoxide (807 mg, 7.20 mmol, 6 eq) in t-BuOH (10 mL) was added trimethylsulfoxonium iodide (1.58 g, 7.20 mmol, 6 eq), the mixture was stirred at 50° C. for 1 h before (3R,5R,8R,9S,10S,13S,14S,17S)-10,13-dimethyl-17-(2-methyloxiran-2-yl)-2,3,4,5,6,7,8,9,11,12,14,15,16,17-tetradecahydro-1H-cyclopenta[a]phenanthren-3-ol (400 mg, 1.20 mmol, 1 eq) was added, and the resulting mixture was stirred at 70° C. for 64 h. The mixture was diluted with water (30 mL), extracted with EtOAc (30 mL×3). The combined organic layer was dried over Na₂SO₄, filtered and concentrated. The residue was purified by flash silica gel chromatography (ISCO®; 20 g SepaFlash® Silica Flash Column, eluent of 0-35% ethyl acetate/petroleum ether gradient @ 30 mL/min) to give the product (250 mg, 60% yield) as a white solid. The product (150 mg) was further purified by prep-TLC (dichloromethane/ethyl acetate=5/1) to give (3R,5R,8R,9S,10S,13S,14S,17S)-10,13-dimethyl-17-(2-methyloxetan-2-yl)-2,3,4,5,6,7,8,9,11,12,14,15,16,17-tetradecahydro-1H-cyclopenta[a]phenanthren-3-ol (20.7 mg, 5.0% yield) as a white solid. LCMS (ESI) m/z, C23H₃₈O₂: calculated 346.29, found [M-OH]⁺: 329.28. ¹H NMR (400 MHz, CDCl₃) δ (ppm) 4.55-4.50 (m, 1H), 4.39-4.33 (m, 1H), 3.68-3.61 (m, 1H), 2.63-2.58 (m, 1H), 2.21-2.09 (m, 2H), 2.05-2.01 (m, 1H), 1.92-1.76 (m, 4H), 1.71-1.64 (m, 2H), 1.57 (s, 3H), 1.55-1.49 (m, 1H), 1.46-1.38 (m, 7H), 1.32-1.05 (m, 7H), 1.01-0.92 (m, 4H), 0.73 (s, 3H). ¹³C NMR (100 MHz, CDCl₃) δ (ppm) 88.99, 71.84, 64.60, 59.50, 56.80, 43.11, 42.04, 40.36, 40.14, 36.41, 35.31, 35.27, 34.56, 33.42, 30.51, 28.33, 27.16, 26.32, 23.93, 23.37, 22.94, 20.56, 12.61.

### Example 5: (3R,5S,8R,9S,10S,13S,14S,17S)-10,13-dimethyl-17-(3-methyloxetan-3-yl)-2,3,4,5,6,7,8,9,11,12,14,15,16,17-tetradecahydro-1H-cyclopenta[a]phenanthren-3-ol

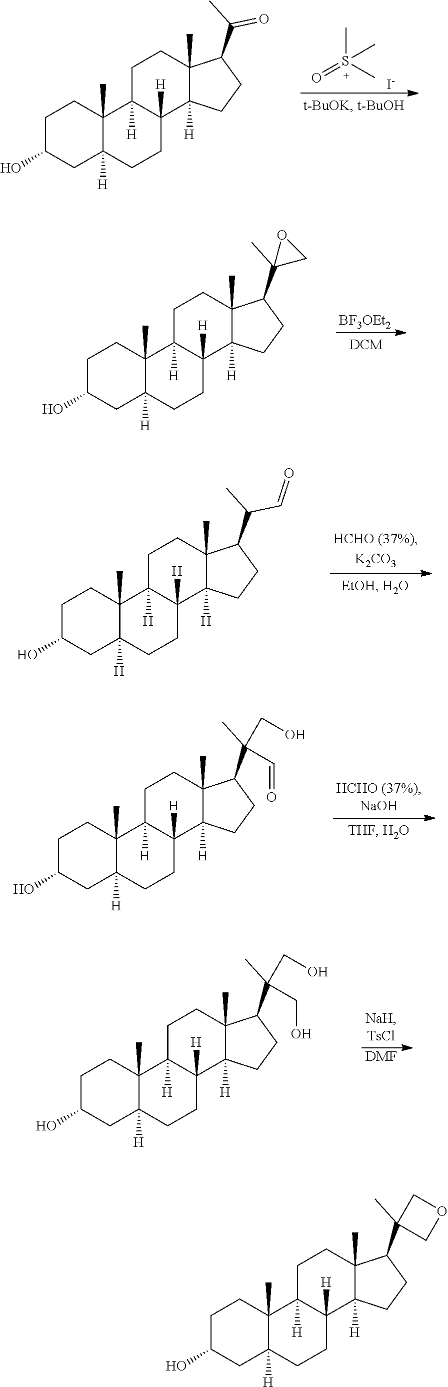

### Preparation of (3R,5S,8R,9S,10S,13S,14S,17S)-10,13-dimethyl-17-(2-methyloxiran-2-yl)-2,3,4,5,6,7,8,9,11,12,14,15,16,17-tetradecahydro-1H-cyclopenta[a]phenanthren-3-ol

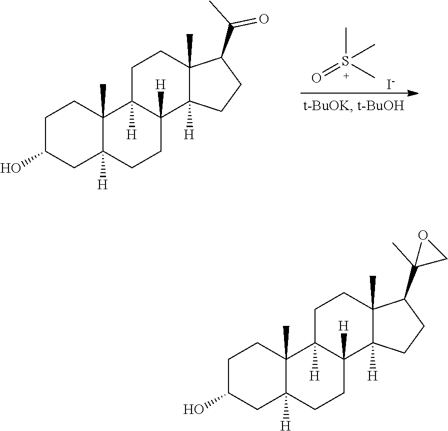

A mixture of t-BuOK (3.17 g, 28.3 mmol, 3 eq) and trimethylsulfoxonium iodide (6.22 g, 28.3 mmol, 3 eq) in t-BuOH (30 mL) was stirred at 50° C. for 1 h before 1-[(3R,5S,8R,9S,10S,13S,14S,17S)-3-hydroxy-10,13-dimethyl-2,3,4,5,6,7,8,9,11,12,14,15,16,17-tetradecahydro-1H-cyclopenta[a]phenanthren-17-yl]ethanone (3.00 g, 9.42 mmol, 1 eq) was added. The resulting mixture was stirred at 50° C. for another 50 h, and then was concentrated and diluted with H₂O (50 mL), extracted with DCM (50 mL×3). The combined organic layers were concentrated. The resulting residue was purified by flash silica gel chromatography (ISCO®; 40 g SepaFlash® Silica Flash Column, eluent with 0-20% ethyl acetate/petroleum ether gradient @ 35 mL/min) to give (3R,5S,8R,9S,10S,13S,14S,17S)-10,13-dimethyl-17-(2-methyloxiran-2-yl)-2,3,4,5,6,7,8,9,11,12,14,15,16,17-tetradecahydro-1H-cyclopenta[a]phenanthren-3-ol (2.10 g, 67% yield) as a white solid.

### Preparation of 2-[(3R,5S,8R,9S,10S,13S,14S,17R)-3-hydroxy-10,13-dimethyl-2,3,4,5,6,7,8,9,11,12,14,15,16,17-tetradecahydro-1H-cyclopenta[a]phenanthren-17-yl]propanal

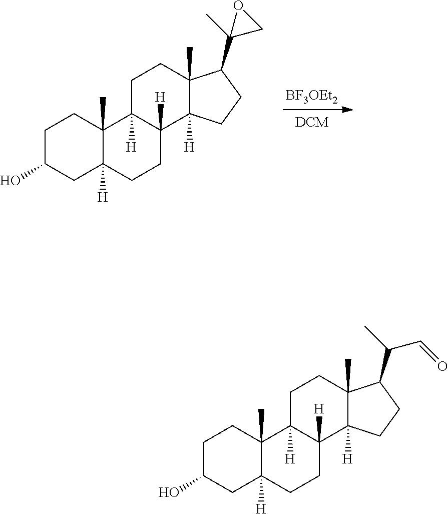

To a solution of (3R,5S,8R,9S,10S,13S,14S,17S)-10,13-dimethyl-17-(2-methyloxiran-2-yl)-2,3,4,5,6,7,8,9,11,12,14,15,16,17-tetradecahydro-1H-cyclopenta[a]phenanthren-3-ol (1.00 g, 3.01 mmol, 1 eq) in DCM (10 mL) was added BF₃.Et₂O (554 mg, 3.91 mmol, 0.48 mL, 1.3 eq) at 0° C. The resulting mixture was stirred at 0° C. for 1 h, and then diluted with H₂O (30 mL) and extracted with DCM (30 mL×3). The combined organic layers were washed with brine (50 mL), dried over Na₂SO₄, filtered and concentrated. The resulting residue was purified by flash silica gel chromatography (ISCO®; 12 g SepaFlash® Silica Flash Column, eluent of 0-20% ethyl acetate/petroleum ether gradient @ 20 mL/min) to give 2-[(3R,5S,8R,9S,10S,13S,14S,17R)-3-hydroxy-10,13-dimethyl-2,3,4,5,6,7,8,9,11,12,14,15,16,17-tetradecahydro-1H-cyclopenta[a]phenanthren-17-yl]propanal (550 mg, 55% yield) as a white solid. ¹H NMR (400 MHz, CDCl₃) δ (ppm) 9.57 (m, 1H), 4.05 (s, 1H), 2.36-2.26 (m, 1H), 1.94-1.81 (m, 1H), 1.69-0.93 (m, 24H), 0.79-0.66 (m, 7H).

### Preparation of 3-hydroxy-2-[(3R,5S,8R,9S,10S,13S,14S,17S)-3-hydroxy-10,13-dimethyl-2,3,4,5,6,7,8,9,11,12,14,15,16,17-tetradecahydro-1H-cyclopenta[a]phenanthren-17-yl]-2-methyl-propanal

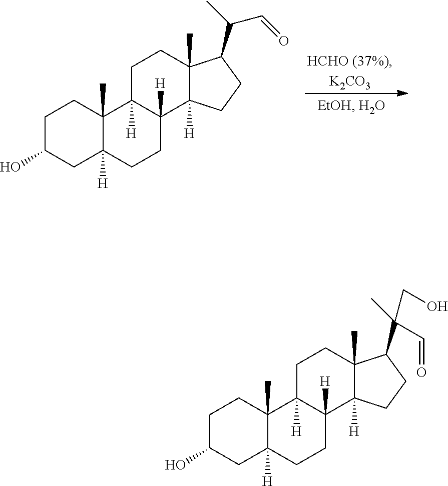

A mixture of 2-[(3R,5S,8R,9S,10S,13S,14S,17R)-3-hydroxy-10,13-dimethyl-2,3,4,5,6,7,8,9,11,12,14,15,16,17-tetradecahydro-1H-cyclopenta[a]phenanthren-17-yl]propanal (480 mg, 1.44 mmol, 1 eq), HCHO (10.9 g, 134 mmol, 10.0 mL, 37% in H₂O, 93 eq) and K₂CO₃ (199 mg, 1.44 mmol, 1 eq) in H₂O (8 mL) and EtOH (8 mL) was degassed and purged with N₂ for 3 times, and then the mixture was stirred at 100° C. for 16 h under N₂ atmosphere. The suspension was filtered, the cake was washed with H₂O (10 mL) and the resulting residue was purified by flash silica gel chromatography (ISCO®; 12 g SepaFlash® Silica Flash Column, eluent with 0-10% methanol/dichloromethane gradient @ 20 mL/min) to give 3-hydroxy-2-[(3R,5S,8R,9S,10S,13S,14S,17S)-3-hydroxy-10,13-dimethyl-2,3,4,5,6,7,8,9,11,12,14,15,16,17-tetradecahydro-1H-cyclopenta[a]phenanthren-17-yl]-2-methyl-propanal (300 mg, 57% yield) as a white solid.

### Preparation of 2-[(3R,5S,8R,9S,10S,13S,14S,17S)-3-hydroxy-10,13-dimethyl-2,3,4,5,6,7,8,9,11,12,14,15,16,17-tetradecahydro-1H-cyclopenta[a]phenanthren-17-yl]-2-methyl-propane-1,3-diol

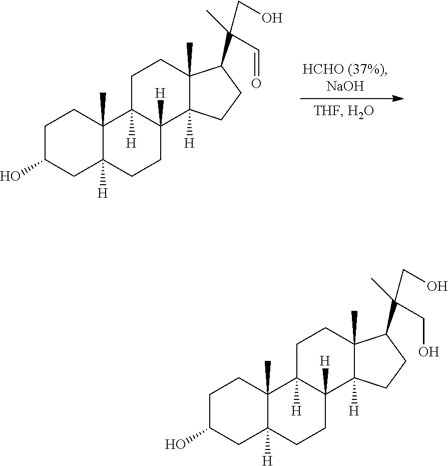

To a mixture of 3-hydroxy-2-[(3R,5S,8R,9S,10S,13S,14S,17S)-3-hydroxy-10,13-dimethyl-2,3,4,5,6,7,8,9,11,12,14,15,16,17-tetradecahydro-1H-cyclopenta[a]phenanthren-17-yl]-2-methyl-propanal (200 mg, 0.55 mmol, 1 eq) in THF (5 mL) and H₂O (5 mL) was added HCHO (4.36 g, 53.3 mmol, 4.0 mL, 37% in H₂O, 97 eq) and NaOH (88 mg, 2.21 mmol, 4 eq). The resulting reaction mixture was stirred at room temperature for 16 h, then concentrated and diluted with H₂O (20 mL). The resulting mixture was filtered, the cake was collected and washed with H₂O (20 mL), then dried under vacuum to give 2-[(3R,5S,8R,9S,10S,13S,14S,17S)-3-hydroxy-10,13-dimethyl-2,3,4,5,6,7,8,9,11,12,14,15,16,17-tetradecahydro-1H-cyclopenta[a]phenanthren-17-yl]-2-methyl-propane-1,3-diol (190 mg, 94% yield) as a white solid.

### Preparation of (3R,5S,8R,9S,10S,13S,14S,17S)-10,13-dimethyl-17-(3-methyloxetan-3-yl)-2,3,4,5,6,7,8,9,11,12,14,15,16,17-tetradecahydro-1H-cyclopenta[a]phenanthren-3-ol

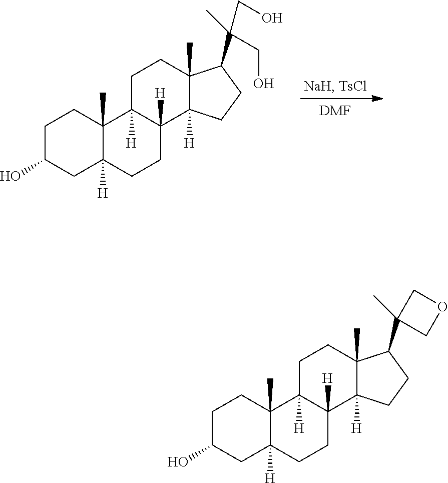

To a solution of 2-[(3R,5S,8R,9S,10S,13S,14S,17S)-3-hydroxy-10,13-dimethyl-2,3,4,5,6,7,8,9,11,12,14,15,16,17-tetradecahydro-1H-cyclopenta[a]phenanthren-17-yl]-2-methyl-propane-1,3-diol (100 mg, 0.274 mmol, 1 eq) in DMF (5 mL) was added NaH (55 mg, 1.37 mmol, 60% in mineral oil, 5 eq). The mixture was stirred at room temperature for 0.5 h before p-toluenesulfonyl chloride (57 mg, 0.301 mmol, 1.1 eq) was added. The resulting mixture was stirred at room temperature for 1 h before another portion of NaH (10.9 mg, 0.274 mmol, 60% in mineral oil, 1 eq) was added, and the mixture was stirred at room temperature for additional 16 h. The reaction mixture was then diluted with H₂O (10 mL) and extracted with EtOAc (10 mL×3). The combined organic layers were washed with brine (20 mL), dried over Na₂SO₄, filtered and concentrated. The crude product was combined with another batch and the combined crude product was purified by flash silica gel chromatography (ISCO®; 12 g SepaFlash® Silica Flash Column, eluted with 0-20% ethyl acetate/petroleum ether gradient @ 20 mL/min). Then the product was recrystallized from EtOAc (10 mL) to give (3R,5S,8R,9S,10S,13S,14S,17S)-10,13-dimethyl-17-(3-methyloxetan-3-yl)-2,3,4,5,6,7,8,9,11,12,14,15,16,17-tetradecahydro-1H-cyclopenta[a]phenanthren-3-ol (67 mg, 32% yield) as a white solid. HRMS (ESI) m/z, C₂₃H₃₈O₂: calculated 346.2875, found (M+H)⁺: 347.2948. ¹H NMR (400 MHz, CDCl₃) δ (ppm) 4.86-4.84 (d, J=8.0 Hz, 1H), 4.59-4.58 (d, J=4.0 Hz, 1H), 4.21-4.20 (d, J=4.0 Hz, 1H), 4.15-4.13 (d, J=8.0 Hz, 1H), 4.05 (s, 1H), 2.05-1.82 (m, 3H), 1.73-1.58 (m, 5H), 1.54-1.43 (m, 8H), 1.39-1.30 (m, 3H), 1.27-1.13 (m, 5H), 1.10-0.89 (m, 2H), 0.77-0.71 (m, 4H), 0.53 (s, 3H). ¹³C NMR (400 MHz, CDCl₃) δ (ppm) 83.58, 79.91, 66.54, 56.39, 55.26, 54.13, 43.45, 41.94, 39.85, 39.07, 36.04, 35.83, 34.98, 32.12, 31.91, 28.97, 28.47, 26.34, 24.30, 24.21, 20.50, 12.39, 11.16.

### Example 6: (3R,5R,8R,9S,10S,13S,14S,17S)-10,13-dimethyl-17-(3-methyloxetan-3-yl)-2,3,4,5,6,7,8,9,11,12,14,15,16,17-tetradecahydro-1H-cyclopenta[a]phenanthren-3-ol

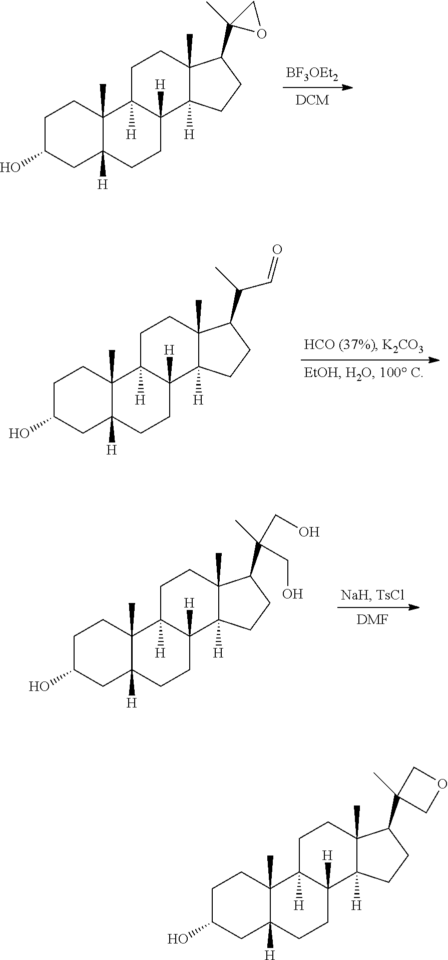

### Preparation of 2-[(3R,5R,8R,9S,10S,13S,14S,17R)-3-hydroxy-10,13-dimethyl-2,3,4,5,6,7,8,9,11,12,14,15,16,17-tetradecahydro-1H-cyclopenta[a]phenanthren-17-yl]propanal

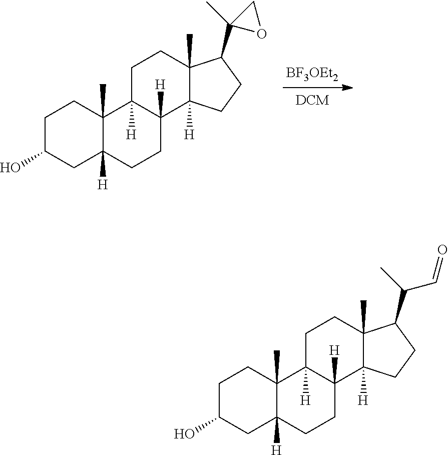

To a solution of (3R,5R,8R,9S,10S,13S,14S,17S)-10,13-dimethyl-17-(2-methyloxiran-2-yl)-2,3,4,5,6,7,8,9,11,12,14,15,16,17-tetradecahydro-1H-cyclopenta[a]phenanthren-3-ol (250 mg, 0.75 mmol, 1 eq) in DCM (1 mL) was added BF₃.Et₂O (149 mg, 1.05 mmol, 0.13 mL, 1.4 eq). The resulting mixture was stirred at 20° C. for 1.5 h. The mixture was quenched by sat. NaHCO₃ (15 mL), extracted with DCM (15 mL×3). The organic layer was washed with brine (20 mL), filtered and concentrated. The residue was purified by flash silica gel chromatography (ISCO®; 4 g SepaFlash® Silica Flash Column, eluent of 0-30% ethyl acetate/petroleum ether gradient @ 25 mL/min) to give 2-[(3R,5R,8R,9S,10S,13S,14S,17R)-3-hydroxy-10,13-dimethyl-2,3,4,5,6,7,8,9,11,12,14,15,16,17-tetradecahydro-1H-cyclopenta[a]phenanthren-17-yl]propanal (120 mg, 48% yield) as a white solid. ¹H NMR (400 MHz, CDCl₃) δ ppm 9.58-9.53 (m, 1H), 3.66-3.51 (m, 1H), 2.38-2.30 (m, 1H), 1.95-1.75 (m, 4H), 1.71-1.59 (m, 3H), 1.54-1.49 (m, 2H), 1.45-1.22 (m, 11H), 1.13-1.10 (m, 4H), 1.04 (d, J=7.0 Hz, 2H), 1.02-0.96 (m, 1H), 0.94-0.92 (m, 3H), 0.70-0.66 (m, 3H).

### Preparation of 2-[(3R,5R,8R,9S,10S,13S,14S,17S)-3-hydroxy-10,13-dimethyl-2,3,4,5,6,7,8,9,11,12,14,15,16,17-tetradecahydro-1H-cyclopenta[a]phenanthren-17-yl]-2-methyl-propane-1,3-diol

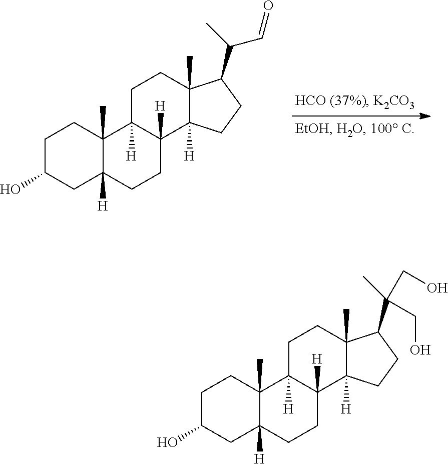

To a mixture of 2-[(3R,5R,8R,9S,10S,13S,14S,17R)-3-hydroxy-10,13-dimethyl-2,3,4,5,6,7,8,9,11,12,14,15,16,17-tetradecahydro-1H-cyclopenta[a]phenanthren-17-yl]propanal (120 mg, 0.36 mmol, 1 eq) in EtOH (2 mL) and H₂O (2 mL) were added HCHO (2.73 g, 33.5 mmol, 2.5 mL, 37% in H₂O, 93 eq) and K₂CO₃ (200 mg, 1.45 mmol, 4 eq). The resulting mixture was stirred at 100° C. for 16 h, then concentrated. The residue was washed with water (4 mL) and dried. The resulting residue was purified by flash silica gel chromatography (ISCO®; 4 g SepaFlash® Silica Flash Column, eluted with 0-10% ethyl acetate/petroleum ether gradient @ 25 mL/min) to give 2-[(3R,5R,8R,9S,10S,13S,14S,17S)-3-hydroxy-10,13-dimethyl-2,3,4,5,6,7,8,9,11,12,14,15,16,17-tetradecahydro-1H-cyclopenta[a]phenanthren-17-yl]-2-methyl-propane-1,3-diol (80 mg, 61% yield) as a white solid.

### Preparation of (3R,5R,8R,9S,10S,13S,14S,17S)-10,13-dimethyl-17-(3-methyloxetan-3-yl)-2,3,4,5,6,7,8,9,11,12,14,15,16,17-tetradecahydro-1H-cyclopenta[a]phenanthren-3-ol

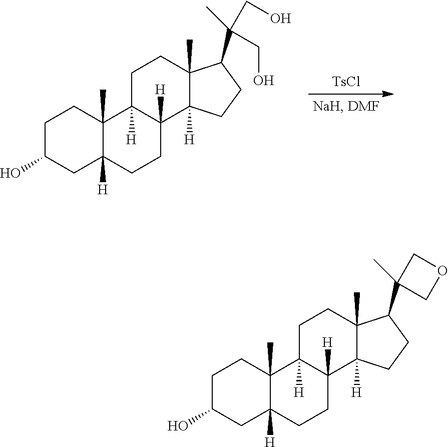

To a solution of 2-[(3R,5R,8R,9S,10S,13S,14S,17S)-3-hydroxy-10,13-dimethyl-2,3,4,5,6,7,8,9,11,12,14,15,16,17-tetradecahydro-1H-cyclopenta[a]phenanthren-17-yl]-2-methyl-propane-1,3-diol (80 mg, 0.22 mmol, 1 eq) in DMF (5 mL) was added NaH (35.1 mg, 0.88 mmol, 60% in mineral oil, 4 eq) at 25° C. The mixture was stirred at 25° C. for 30 min before 4-methylbenzenesulfonyl chloride (46.0 mg, 0.24 mmol, 1.1 eq) was added. The resulting mixture was stirred at 25° C. for 64 h. The mixture was diluted with EtOAc (50 mL), washed with water (20 mL×2), dried over Na₂SO₄, filtered and concentrated. The residue was purified by flash silica gel chromatography (ISCO®; 12 g SepaFlash® Silica Flash Column, eluent of 0-20% ethyl acetate/petroleum ether gradient @ 20 mL/min) to give (3R,5R,8R,9S,10S,13S,14S,17S)-10,13-dimethyl-17-(3-methyloxetan-3-yl)-2,3,4,5,6,7,8,9,11,12,14,15,16,17-tetradecahydro-1H-cyclopenta[a]phenanthren-3-ol (32.0 mg, 42% yield) as a white solid. LCMS (ESI) m/z, C23H₃₈O₂: calculated 346.29, found [M+H]⁺: 347.29. ¹H NMR (400 MHz, CDCl₃) δ (ppm) 4.85 (d, J=8.0 Hz, 1H), 4.59 (d, J=8.0 Hz, 1H), 4.21 (d, J=4.0 Hz, 1H), 4.14 (d, J=4.0 Hz, 1H), 3.66-3.61 (m, 1H), 2.05-1.66 (m, 8H), 1.57-1.45 (m, 4H), 1.47-1.34 (m, 7H), 1.31-1.07 (m, 7H), 1.04-0.90 (m, 4H), 0.52 (s, 3H). ¹³C NMR (100 MHz, CDCl₃) δ (ppm) 83.59, 79.88, 71.79, 56.39, 55.35, 43.55, 42.00, 41.93, 40.31, 40.03, 36.38, 35.35, 35.28, 34.54, 30.48, 27.10, 26.36, 24.41, 24.30, 23.32, 20.56, 12.35.

### Example 7: (3R,5R,8R,9R,10S,13S,14S,17S)-3,13-dimethyl-17-(3-methyltetrahydrofuran-3-yl)-2,4,5,6,7,8,9,10,11,12,14,15,16,17-tetradecahydro-1H-cyclopenta[a]phenanthren-3-ol

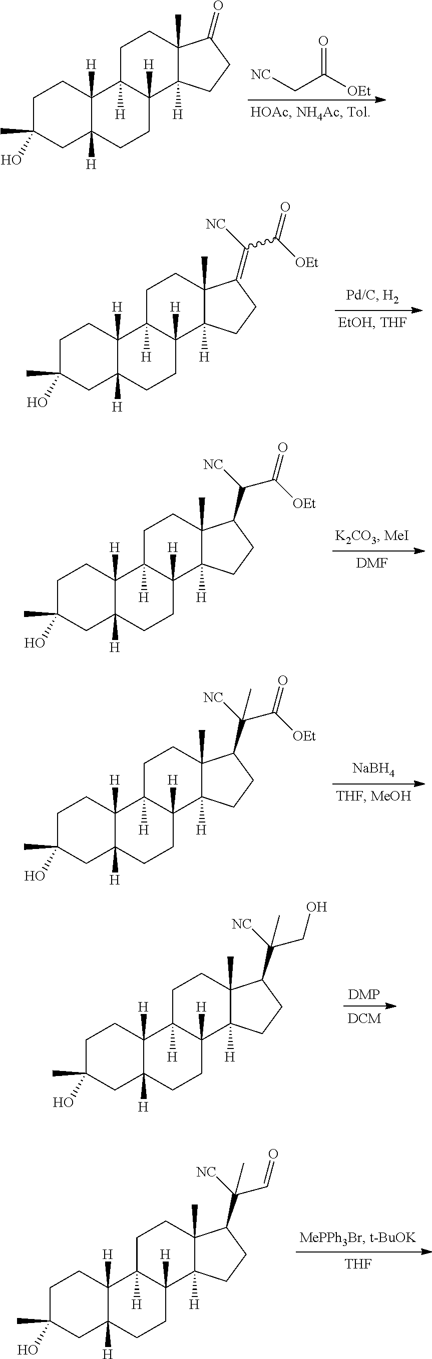

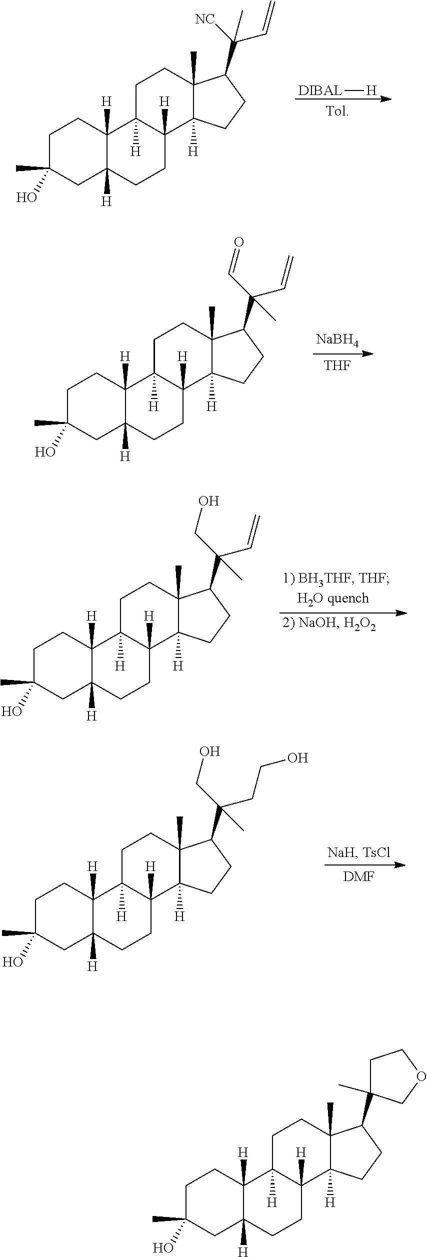

### Preparation of ethyl 2-cyano-2-((3R,5R,8R,9R,10S,13S,14S)-3-hydroxy-3,13-dimethylhexadecahydro-17H-cyclopenta[a]phenanthren-17-ylidene)acetate

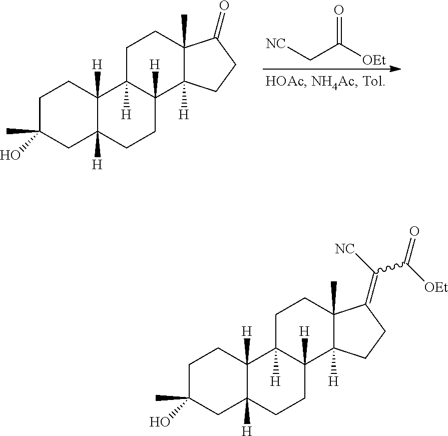

To a mixture of (3R,5R,8R,9R,10S,13S,14S)-3-hydroxy-3,13-dimethyl-1,2,4,5,6,7,8,9,10,11,12,14,15,16-tetradecahydrocyclopenta[a]phenanthren-17-one (7.00 g, 24.1 mmol, 1 eq) in toluene (200 mL) was added ethyl 2-cyanoacetate (13.6 g, 120 mmol, 5 eq), NH₄OAc (5.57 g, 72.3 mmol, 3 eq) and HOAc (26.2 g, 436 mmol, 25 mL, 18.1 eq). The resulting mixture was stirred at 135° C. for 16 h under a Dean-Stark water separator. The reaction mixture was then concentrated and diluted with EtOAc (400 mL), washed with saturated aqueous NaHCO₃ (200 mL×2) and brine (200 mL), the organic layer was dried over Na₂SO₄, filtered and concentrated. The residue was purified by flash silica gel chromatography (ISCO®; 220 g SepaFlash® Silica Flash Column, eluted with 0-10% ethyl acetate/dichloromethane gradient @ 80 mL/min) to give ethyl 2-cyano-2-((3R,5R,8R,9R,10S,13S,14S)-3-hydroxy-3,13-dimethylhexadecahydro-17H-cyclopenta[a]phenanthren-17-ylidene)acetate (7.00 g, 73.3% yield) as a white solid.

### Preparation of ethyl 2-cyano-2-[(3R,5R,8R,9R,10S,13S,14S,17R)-3-hydroxy-3,13-dimethyl-2,4,5,6,7,8,9,10,11,12,14,15,16,17-tetradecahydro-1H-cyclopenta[a]phenanthren-17-yl]acetate

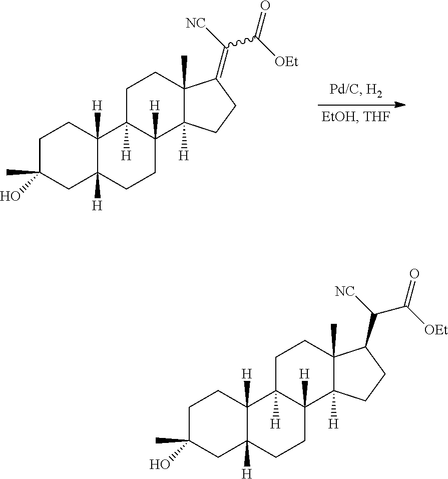

To a solution of ethyl 2-cyano-2-((3R,5R,8R,9R,10S,13S,14S)-3-hydroxy-3,13-dimethylhexadecahydro-17H-cyclopenta[a]phenanthren-17-ylidene)acetate (7.02 g, 18.2 mmol, 1 eq) in EtOH (25 mL) and THF (75 mL) was added Pd/C (800 mg, 10 wt % loading), and then the mixture was stirred under H₂ (15 psi) at 20° C. for 6 h. The resulting reaction mixture was filtered and the filtrate was concentrated to give ethyl 2-cyano-2-[(3R,5R,8R,9R,10S,13S,14S,17R)-3-hydroxy-3,13-dimethyl-2,4,5,6,7,8,9,10,11,12,14,15,16,17-tetradecahydro-1H-cyclopenta[a]phenanthren-17-yl]acetate (6.84 g, 96.8% yield) as white solid which was used directly for next step without further purification. ¹H NMR (400 MHz, CDCl₃) δ (ppm) 4.28-4.23 (m, 2H) 3.41-3.26 (m, 1H) 2.21-2.05 (m, 2H) 1.81-1.79 (m, 6H) 1.42-1.06 (m, 22H) 0.77-0.76 (d, J=4.0 Hz, 3H).

### Preparation of ethyl 2-cyano-2-[(3R,5R,8R,9R,10S,13S,14S,175)-3-hydroxy-3,13-dimethyl-2,4,5,6,7,8,9,10,11,12,14,15,16,17-tetradecahydro-1H-cyclopenta[a]phenanthren-17-yl]propanoate

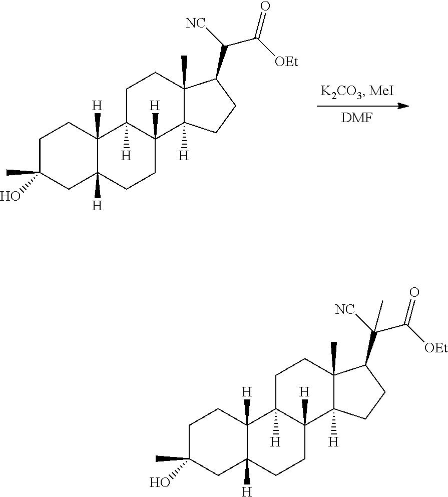

To a solution of ethyl 2-cyano-2-[(3R,5R,8R,9R,10S,13S,14S,17R)-3-hydroxy-3,13-dimethyl-2,4,5,6,7,8,9,10,11,12,14,15,16,17-tetradecahydro-1H-cyclopenta[a]phenanthren-17-yl]acetate (500 mg, 1.29 mmol, 1 eq) in DMF (5 mL) was added K₂CO₃ (356 mg, 2.58 mmol, 2 eq) and MeI (915 mg, 6.45 mmol, 5 eq). The resulting mixture was stirred at room temperature for 3 h, then diluted with EtOAc (15 mL), washed with water (10 mL×3), brine (10 mL). The organic layer was dried over Na₂SO₄, filtered and concentrated under reduced pressure. The residue was purified by flash silica gel chromatography (ISCO®; 4 g SepaFlash® Silica Flash Column, eluted with 0-30% ethyl acetate/petroleum ether gradient @ 20 mL/min) to give ethyl 2-cyano-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propanoate (450 mg, 86.9% yield) as a white solid.

### Preparation of 3-hydroxy-2-[(3R,5R,8R,9R,10S,13S,14S,175)-3-hydroxy-3,13-dimethyl-2,4,5,6,7,8,9,10,11,12,14,15,16,17-tetradecahydro-1H-cyclopenta[a]phenanthren-17-yl]-2-methyl-propanenitrile

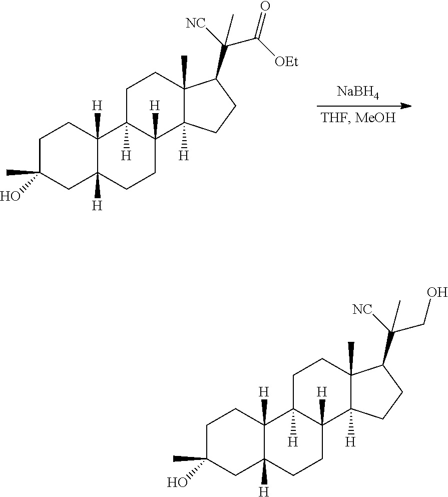

To a mixture of ethyl 2-cyano-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propanoate (430 mg, 1.07 mmol, 1 eq) and NaBH₄ (486 mg, 12.8 mmol, 12 eq) in THF (4 mL) was added MeOH (2 mL). The resulting mixture was stirred at 75° C. for 16 h. The reaction mixture was quenched with water (8 mL) and extracted with EtOAc (10 mL×2). The combined organic layer was dried over Na₂SO₄, filtered and concentrated. The residue was purified by flash silica gel chromatography (ISCO®; 4 g SepaFlash® Silica Flash Column, eluted with 0-80% ethyl acetate/petroleum ether gradient @ 20 mL/min) to give 3-hydroxy-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]-2-methyl-propanenitrile (360 mg, 93.5% yield) as white solid.

### Preparation of 2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]-2-methyl-3-oxo-propanenitrile

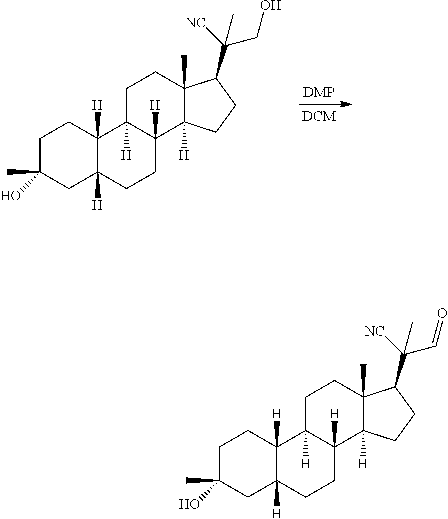

To a solution of 3-hydroxy-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]-2-methyl-propanenitrile (200 mg, 0.56 mmol, 1 eq) in DCM (3 mL) was added Dess-Martin periodinane (306 mg, 0.72 mmol, 1.3 eq). The resulting mixture was stirred at room temperature for 2 h. The reaction mixture was quenched by saturated Na₂SO₃ (3 mL) and then extracted with DCM (5 mL×2). The combined organic layers were washed with H₂O (10 mL), dried over Na₂SO₄, filtered and concentrated under reduced pressure. The residue was purified by flash silica gel chromatography (ISCO®; 4 g SepaFlash® Silica Flash Column, eluted with 0-50% ethyl acetate/petroleum ether gradient @ 20 mL/min) to give 2-[(3R,5R,8R,9R, 10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]-2-methyl-3-oxo-propanenitrile (180 mg, 90.5% yield) as a white solid.

### Preparation of 2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]-2-methyl-but-3-enenitrile

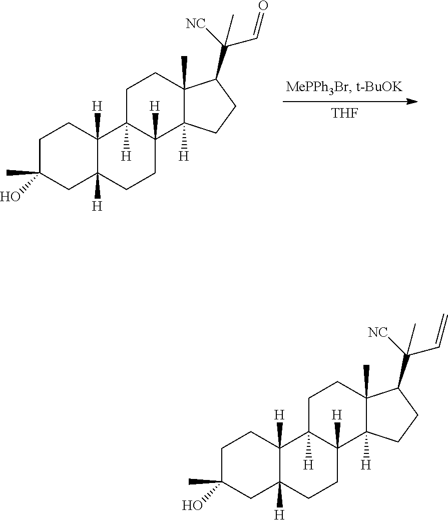

A mixture of t-BuOK (113 mg, 1.01 mmol, 2 eq) and methyl(triphenyl)phosphonium;bromide (360 mg, 1.01 mmol, 2 eq) in THF (5 mL) was stirred at 30° C. for 1 h before a mixture of 2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]-2-methyl-3-oxo-propanenitrile (180 mg, 0.50 mmol, 1 eq) in THF (3 mL) was added. The resulting mixture was stirred at 30° C. for another 2 h, then diluted with EtOAc (15 mL) and washed with water (5 mL×3). The organic layer was washed with brine (20 mL), dried over Na₂SO₄, filtered and concentrated. The residue was purified by flash silica gel chromatography (ISCO®; 4 g SepaFlash® Silica Flash Column, eluted with 0-25% ethyl acetate/petroleum ether gradient @ 20 mL/min) to give 2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren yl]-2-methyl-but-3-enenitrile (160 mg, 89.4% yield) as a white solid. ¹H NMR (400 MHz, CDCl₃) δ (ppm) 5.70-5.63 (m, 1H), 5.56-5.52 (d, J=17.2 Hz, 1H), 5.18-5.16 (d, J=10.0 Hz, 1H), 1.98-1.79 (m, 8H), 1.65-1.55 (m, 3H), 1.44-0.99 (m, 20H), 0.86 (s, 3H).

### Preparation of 2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]-2-methyl-but-3-enal

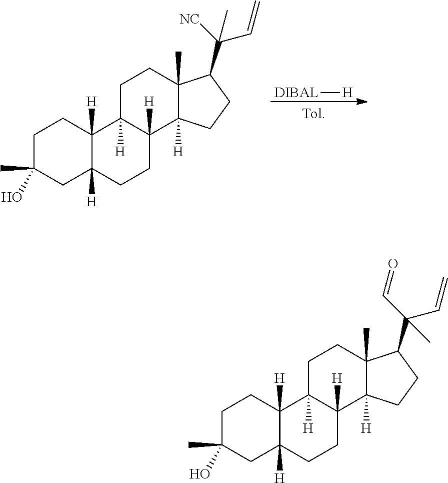

To a solution of 2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]-2-methyl-but-3-enenitrile (120 mg, 0.34 mmol, 1 eq) in toluene (3 mL) was added DIBAL-H (1.0 M in toluene, 1.01 mL, 3 eq) at 0° C. The resulting mixture was stirred at 0° C. for 3 h, and then quenched with saturated aqueous NH₄Cl (3 mL) and extracted with DCM (5 mL×3). The organic layers were combined, washed with brine (10 mL), dried over Na₂SO₄, filtered, and concentrated under reduced pressure. The residue was purified by flash silica gel chromatography (ISCO®; 4 g SepaFlash® Silica Flash Column, eluted with 0-20% ethyl acetate/petroleum ether gradient @ 20 mL/min) to give 2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]-2-methyl-but-3-enal (100 mg, 82.6% yield) as a white solid.

### Preparation of (3R,5R,8R,9R,10S,13S,14S,17S)-17-[1-(hydroxymethyl)-1-methyl-allyl]-3,13-dimethyl-2,4,5,6,7,8,9,10,11,12,14,15,16,17-tetradecahydro-1H-cyclopenta[a]phenanthren-3-ol

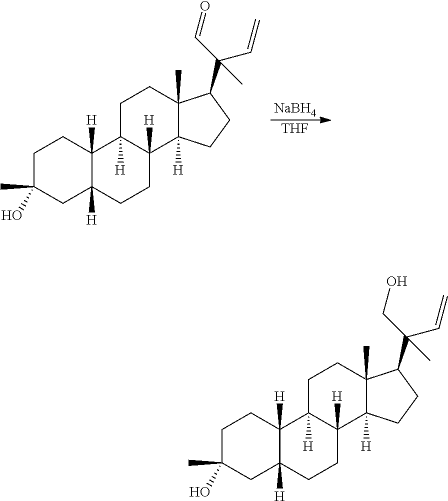

To a solution of 2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]-2-methyl-but-3-enal (110 mg, 0.31 mmol, 1 eq) in THF (2 mL) was added NaBH₄ (58.0 mg, 1.55 mmol, 5 eq). The resulting mixture was stirred at 20° C. for 2 h. The reaction mixture was then diluted with water (2 mL) and extracted with EtOAc (5 mL×2). The organic layers were combined, washed with brine, dried over Na₂SO₄, filtered and concentrated under reduced pressure. The residue was purified by flash silica gel chromatography (ISCO®; 4 g SepaFlash® Silica Flash Column, eluted with 0-40% ethyl acetate/petroleum ether gradient @ 20 mL/min) to give (3R,5R,8R,9R,10S,13S,14S,17S)-17-[1-(hydroxymethyl)-1-methyl-allyl]-3,13-dimethyl-2,4,5,6,7,8,9,10,11,12,14,15,16,17-tetradecahydro-1H-cyclopenta[a]phenanthren-3-ol (70 mg, 63.3% yield) as a white solid.

### Preparation of 2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]-2-methyl-butane-1,4-diol

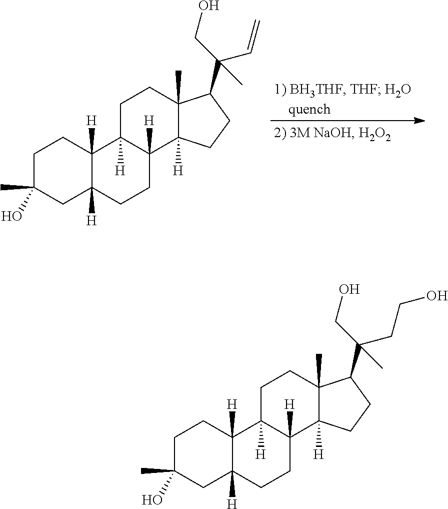

To a solution of (3R,5R,8R,9R,10S,13S,14S,17S)-17-[1-(hydroxymethyl)-1-methyl-allyl]-3,13-dimethyl-2,4,5,6,7,8,9,10,11,12,14,15,16,17-tetradecahydro-1H-cyclopenta[a]phenanthren-3-ol (40 mg, 0.11 mmol, 1 eq) in THF (3 mL) was added BH₃.THF (1.0 M in THF, 0.55 mL, 5 eq) at 0° C. The resulting mixture was stirred at 20° C. for 4 h, and then quenched by H₂O (0.5 mL). To this resulting mixture was sequentially added NaOH (3.0 M in H₂O, 0.4 mL, 11 eq) and H₂O₂ (472 mg, 4.16 mmol, 0.4 mL, 30% in H₂O, 38 eq), and the mixture was stirred at 20° C. for another 16 h. The resulting mixture was diluted with saturated Na₂SO₃ (2 mL) and extracted with EtOAc (8 mL×3). The organic layers were combined, washed with brine (10 mL), dried over Na₂SO₄, filtered, and concentrated under reduced pressure. The residue was purified by prep-TLC (petroleum ether/ethyl acetate=1/1) to give 2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]-2-methyl-butane-1,4-diol (20 mg, 47.6% yield) as a colorless oil. ¹H NMR (400 MHz, CDCl₃) δ (ppm) 3.73-3.66 (m, 3H), 3.51-3.49 (d, J=11.2 Hz, 1H), 2.73 (s, 1H), 1.96-0.94 (m, 33H), 0.79 (s, 3H).

### Preparation of (3R,5R,8R,9R,10S,13S,14S,17S)-3,13-dimethyl-17-(3-methyltetrahydrofuran-3-yl)-2,4,5,6,7,8,9,10,11,12,14,15,16,17-tetradecahydro-1H-cyclopenta[a]phenanthren-3-ol

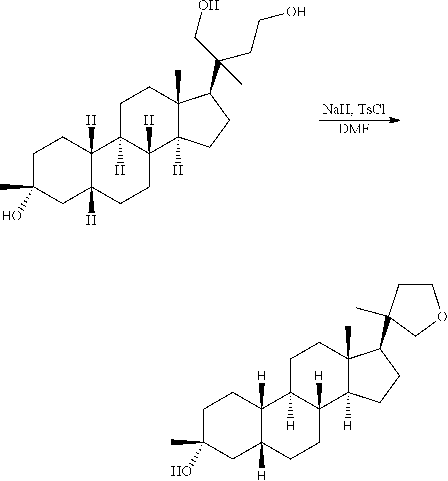

To a solution of 2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]-2-methyl-butane-1,4-diol (20 mg, 0.053 mmol, 1 eq) in DMF (1 mL) was added NaH (21.1 mg, 0.53 mmol, 60% in mineral oil, 10 eq). The mixture was stirred at 20° C. for 1 h before 4-methylbenzenesulfonyl chloride (15.1 mg, 0.079 mmol, 1.5 eq) was added and the mixture was stirred for another 2 h. The reaction mixture was then quenched by water (1 mL) and extracted with EtOAc (5 mL×2). The combined organic layers were washed with H₂O (3 mL), brine (3 mL), dried over Na₂SO₄, filtered, and concentrated under reduced pressure. The residue was purified by prep-TLC (petroleum ether/ethyl acetate=2/1) to give (3R,5R,8R,9R,10S,13S,14S,17S)-3,13-dimethyl-17-(3-methyltetrahydrofuran-3-yl)-2,4,5,6,7,8,9,10,11,12,14,15,16,17-tetradecahydro-1H-cyclopenta[a]phenanthren-3-ol (6.1 mg, 32.0% yield) as a white solid. LCMS (ESI) m/z, C₂₄H₄₀O₂: calculated 360.57, found [M-OH]⁺: 343.3. ¹H NMR (400 MHz, CDCl₃) δ (ppm) 3.89-3.79 (m, 2H) 3.52-3.49 (m, 2H) 1.86-1.79 (m, 5H) 1.64-1.61 (m, 5H) 1.42-1.07 (m, 23H) 0.74 (s, 3H). ¹³C NMR (100 MHz, CDCl₃) δ (ppm) 79.39, 72.05, 67.42, 57.80, 55.28, 45.54, 43.91, 41.27, 41.21, 40.36, 40.06, 38.20, 37.72, 34.76, 34.57, 31.46, 26.44, 25.97, 25.54, 25.43, 23.94, 23.87, 23.83, 14.27.

### Example 8: (3R,5S,8R,9S,10S,13S,14S,17S)-10,13-dimethyl-17-(2-methyltetrahydrofuran-2-yl)-2,3,4,5,6,7,8,9,11,12,14,15,16,17-tetradecahydro-1H-cyclopenta[a]phenanthren-3-ol

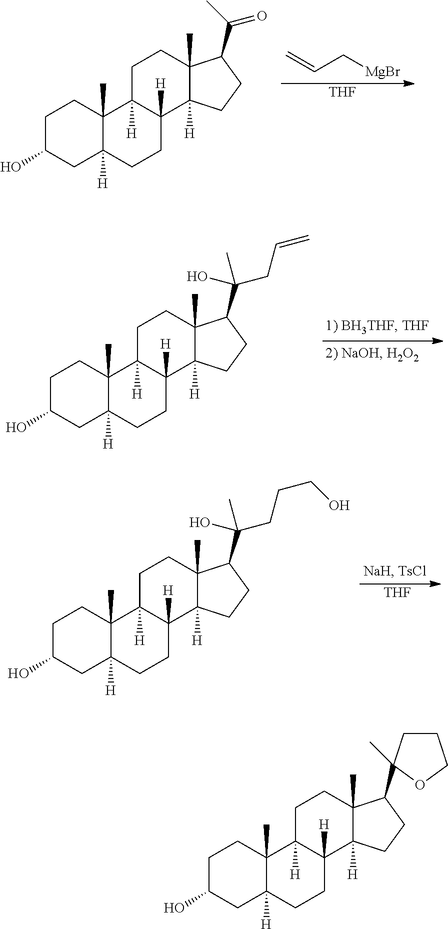

### Preparation of (3R,5S,8R,9S,10S,13S,14S,17S)-17-(1-hydroxy-1-methyl-but-3-enyl)-10,13-dimethyl-2,3,4,5,6,7,8,9,11,12,14,15,16,17-tetradecahydro-1H-cyclopenta[a]phenanthren-3-ol

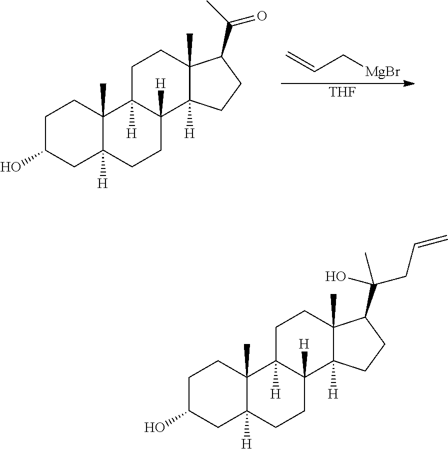

To a solution of 1-[(3R,5S,8R,9S,10S,13S,14S,17S)-3-hydroxy-10,13-dimethyl-2,3,4,5,6,7,8,9,11,12,14,15,16,17-tetradecahydro-1H-cyclopenta[a]phenanthren-17-yl]ethanone (1.00 g, 3.14 mmol, 1 eq) in THF (15 mL) was added allyl(bromo)magnesium (1.0 M in ether, 5.0 mL, 1.6 eq) dropwise at 0° C. The resulting mixture was warmed to room temperature and stirred for 20 h. The reaction mixture was then diluted with water (15 mL) and extracted with EtOAc (20 mL×2). The organic layers were combined, washed with brine (10 mL), dried over Na₂SO₄, filtered, and concentrated under reduced pressure. The residue was purified by flash silica gel chromatography (ISCO®; 12 g SepaFlash® Silica Flash Column, eluted with 0-10% ethyl acetate/petroleum ether gradient @ 20 mL/min) to give (3R,5S,8R,9S,10S,13S,14S,175)-17-(1-hydroxy-1-methyl-but-3-enyl)-10,13-dimethyl-2,3,4,5,6,7,8,9,11,12,14,15,16,17-tetradecahydro-1H-cyclopenta[a]phenanthren-3-ol (340 mg, 30.0% yield) as a white solid.

### Preparation of 4-[(3R,5S,8R,9S,10S,13S,14S,17S)-3-hydroxy-10,13-dimethyl-2,3,4,5,6,7,8,9,11,12,14,15,16,17-tetradecahydro-1H-cyclopenta[a]phenanthren-17-yl]pentane-1,4-diol

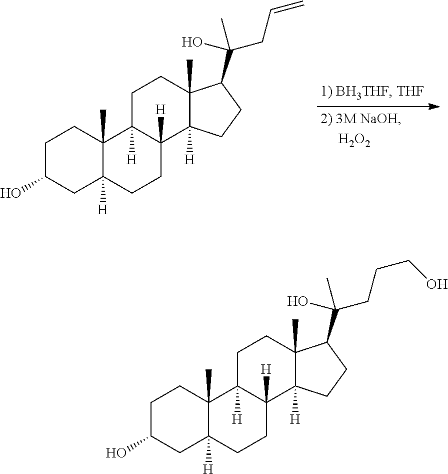

To a solution of (3R,5S,8R,9S,10S,13S,14S,17S)-17-(1-hydroxy-1-methyl-but-3-enyl)-10,13-dimethyl-2,3,4,5,6,7,8,9,11,12,14,15,16,17-tetradecahydro-1H-cyclopenta[a]phenanthren-3-ol (130 mg, 0.36 mmol, 1 eq) in THF (5 mL) was added BH₃.THF (1.0 M in THF, 1.08 mL, 3 eq) at 0° C. The resulting mixture was stirred at room temperature for 3 h before NaOH (3.0 M in water, 1.32 mL, 11 eq) and H₂O₂ (1.53 g, 13.5 mmol, 1.38 mL, 30% in H₂O, 37.5 eq) was added, and the mixture was stirred at room temperature for another 16 h. The reaction was quenched with addition of saturated aqueous Na₂SO₃ (5 mL) and extracted with EtOAc (15 mL×3). The combined organic layers were washed with water (100 mL), dried over Na₂SO₄, filtered, and concentrated under reduced pressure to give 4-[(3R,5S,8R,9S,10S,13S,14S,17S)-3-hydroxy-10,13-dimethyl-2,3,4,5,6,7,8,9,11,12,14,15,16,17-tetradecahydro-1H-cyclopenta[a]phenanthren-17-yl]pentane-1,4-diol (110 mg, crude) as a white solid which was used directly for next step without further purification.

### Preparation of (3R,5S,8R,9S,10S,13S,14S,17S)-10,13-dimethyl-17-(2-methyltetrahydrofuran-2-yl)-2,3,4,5,6,7,8,9,11,12,14,15,16,17-tetradecahydro-1H-cyclopenta[a]phenanthren-3-ol

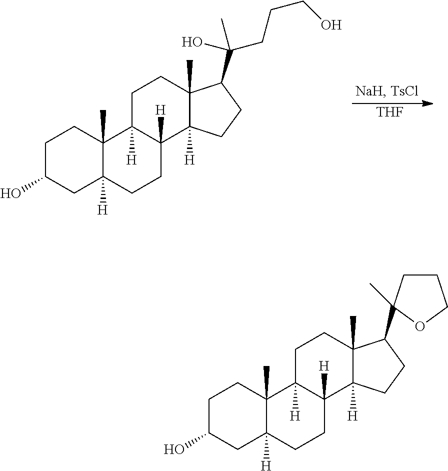

To a solution of 4-[(3R,5S,8R,9S,10S,13S,14S,17S)-3-hydroxy-10,13-dimethyl-2,3,4,5,6,7,8,9,11,12,14,15,16,17-tetradecahydro-1H-cyclopenta[a]phenanthren-17-yl]pentane-1,4-diol (100 mg, 0.26 mmol, 1 eq) in THF (5 mL) was added NaH (31.7 mg, 0.79 mmol, 60% in mineral oil, 3 eq). The mixture was stirred at 25° C. for 1 h before 4-methylbenzenesulfinyl chloride (59.3 mg, 0.31 mmol, 1.2 eq) was added. The resulting mixture was stirred for another 1 h at 25° C. before another portion of NaH (31.7 mg, 0.79 mmol, 60% in mineral oil, 3 eq) was added, and the mixture was stirred at 25° C. for additional 48 h. The reaction mixture was quenched by water (2 mL), adjusted to pH˜8 with aqueous 1.0 M HCl, and then extracted with EtOAc (10 mL×2). The organic layers were combined, washed with brine (15 mL), dried over Na₂SO₄, filtered, and concentrated. The resulting residue was purified by flash silica gel chromatography (ISCO®; 4 g SepaFlash® Silica Flash Column, eluted with 0-15% ethyl acetate/petroleum ether gradient @ 15 mL/min) to give (3R,5S,8R,9S,10S,13S,14S,17S)-10,13-dimethyl-17-(2-methyltetrahydrofuran-2-yl)-2,3,4,5,6,7,8,9,11,12,14,15,16,17-tetradecahydro-1H-cyclopenta[a]phenanthren-3-ol (11.2 mg, 11.4% yield) as a white solid. LCMS (ESI) m/z, C₂₄H₄₀O₂: calculated 360.57, found (M-OH)⁺: 343.3. ¹H NMR (400 MHz, CDCl₃) δ (ppm) 4.05 (s, 1H) 3.92-3.88 (m, 1H) 3.79-3.77 (m, 1H) 2.07-2.03 (m, 1H) 1.87-1.60 (m, 10H) 1.54-0.95 (m, 19H) 0.79-0.74 (m, 7H). ¹³C NMR (100 MHz, CDCl₃) δ (ppm) 84.97, 68.26, 66.64, 59.50, 56.69, 54.38, 42.93, 40.29, 39.16, 37.99, 36.09, 35.91, 35.05, 32.19, 31.93, 29.03, 28.58, 26.59, 25.17, 23.77, 22.98, 20.66, 13.45, 11.20.

### Example 9: (3R,5R,8R,9R,10S,13S,14S,17S)-17-[3-(cyclopropylmethyl)oxetan-3-yl]-3,13-dimethyl-2,4,5,6,7,8,9,10,11,12,14,15,16,17-tetradecahydro-1H-cyclopenta[a]phenanthren-3-ol

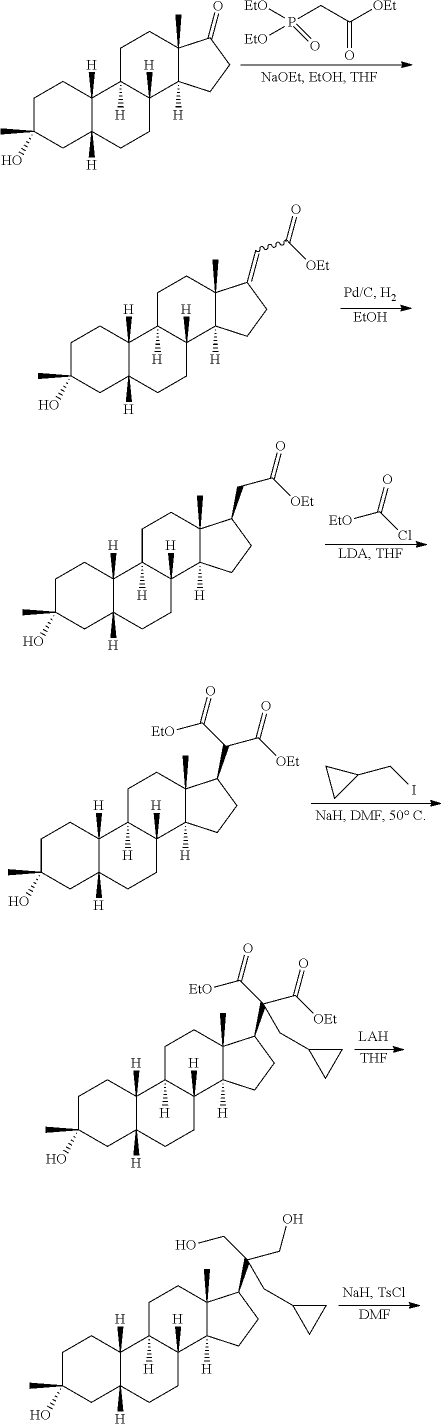

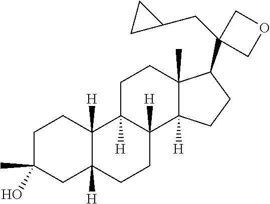

### Preparation of ethyl 2-((3R,5R,8R,9R,10S,13S,14S)-3-hydroxy-3,13-dimethylhexadecahydro-17H-cyclopenta[a]phenanthren-17-ylidene)acetate

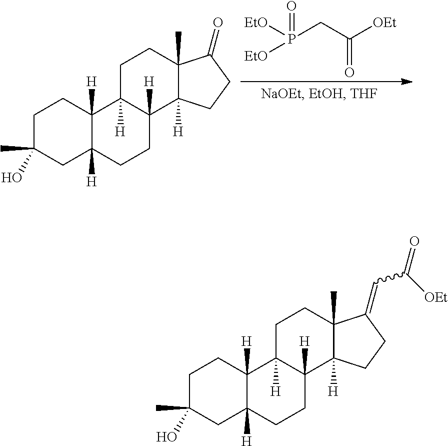

To a solution of (3R,5R,8R,9R,10S,13S,14S)-3-hydroxy-3,13-dimethyl-1,2,4,5,6,7,8,9,10,11,12,14,15,16-tetradecahydrocyclopenta[a]phenanthren-17-one (2.00 g, 6.89 mmol, 1 eq) in THF (20 mL) and ethanol (20 mL) was added sodium ethanolate (1.5 M in EtOH, 45.9 mL, 10 eq) and ethyl 2-diethoxyphosphorylacetate (15.4 g, 68.9 mmol, 13.7 mL, 10 eq). The resulting mixture was stirred at 85° C. for 16 h, and then was concentrated. The resulting residue was diluted with EtOAc (100 mL), washed with 1.0 M HCl in H₂O (50 mL), saturated aqueous NaHCO₃ (50 mL), brine (50 mL), dried over Na₂SO₄, filtered, and concentrated. The residue was purified by flash silica gel chromatography (ISCO®; 80 g SepaFlash® Silica Flash Column, eluent of 0-20% ethyl acetate/petroleum ether gradient @ 60 mL/min) to give ethyl 2-((3R,5R,8R,9R,10S,13S,145)-3-hydroxy-3,13-dimethylhexadecahydro-17H-cyclopenta[a]phenanthren-17-ylidene)acetate (2.40 g, 96.6% yield) as a white solid.

### Preparation of ethyl 2-[(3R,5R,8R,9R,10S,13R,14S,17R)-3-hydroxy-3,13-dimethyl-2,4,5,6,7,8,9,10,11,12,14,15,16,17-tetradecahydro-1H-cyclopenta[a]phenanthren-17-yl]acetate

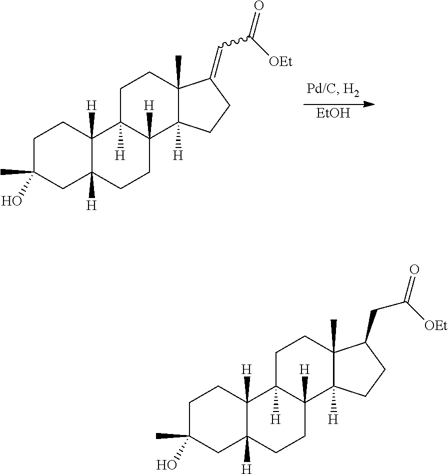

To a solution of ethyl 2-((3R,5R,8R,9R,10S,13S,14S)-3-hydroxy-3,13-dimethylhexadecahydro-17H-cyclopenta[a]phenanthren-17-ylidene)acetate (3.00 g, 8.32 mmol, 1 eq) in EtOH (40 mL) was added Pd/C (400 mg, 10 wt % loading) under N₂ atmosphere, and then the mixture was stirred under H₂ (15 psi) at room temperature for 18 h. The resulting reaction mixture was filtered and the filtrate was concentrated to give ethyl 2-[(3R,5R,8R,9R,10S,13R,14S,17R)-3-hydroxy-3,13-dimethyl-2,4,5,6,7,8,9,10,11,12,14,15,16,17-tetradecahydro-1H-cyclopenta[a]phenanthren-17-yl]acetate (3.00 g, 99.4% yield) as a colorless oil. ¹H NMR (400 MHz, CDCl₃) δ (ppm) 4.12 (q, J=6.8 Hz, 2H), 2.36 (dd, J=4.8, 14.4 Hz, 1H), 2.11 (dd, J=9.6, 14.4 Hz, 1H), 1.97-1.75 (m, 5H), 1.67-1.60 (m, 3H), 1.50-1.37 (m, 6H), 1.36-1.20 (m, 11H), 1.18-0.97 (m, 6H), 0.60 (s, 3H).

### Preparation of diethyl 2-[(3R,5R,8R,9R,10S,13S,14S,17R)-3-hydroxy-3,13-dimethyl-2,4,5,6,7,8,9,10,11,12,14,15,16,17-tetradecahydro-1H-cyclopenta[a]phenanthren-17-yl]propanedioate

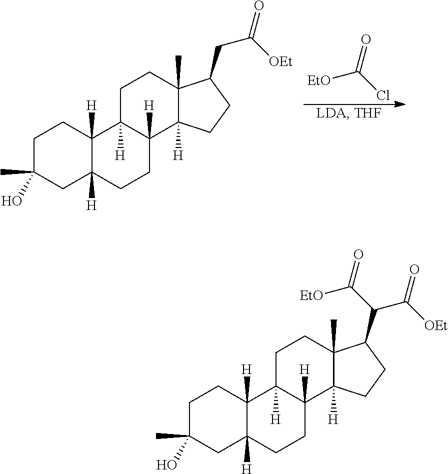

To a solution of DIPA (1.40 g, 13.8 mmol, 1.95 mL, 2.5 eq) in THF (40 mL) was added dropwise n-BuLi (2.5 M in hexanes, 5.52 mL, 2.5 eq) at 0° C. The mixture was stirred at 0° C. for 0.5 h before ethyl 2-[(3R,5R,8R,9R,10S,13R,14S,17R)-3-hydroxy-3,13-dimethyl-2,4,5,6,7,8,9,10,11,12,14,15,16,17-tetradecahydro-1H-cyclopenta[a]phenanthren-17-yl]acetate (2.00 g, 5.52 mmol, 1 eq) and N-[bis(dimethylamino)phosphoryl]-N-methyl-methanamine (1.0 M, 5.52 mL, 1 eq) were added dropwise at −78° C. The resulting mixture was stirred at −78° C. for additional 0.5 h before ethyl carbonochloridate (1.22 g, 11.3 mmol, 1.07 mL, 2.04 eq) was added dropwise at −78° C., and then the mixture was stirred at −78° C. for 4 h. The reaction mixture was quenched with aqueous saturated NH₄Cl (50 mL) and extracted with EtOAc (50 mL×2). The organic layers were combined, washed with brine (50 mL), dried over Na₂SO₄, filtered, and concentrated. The residue was purified by flash silica gel chromatography (ISCO®; 80 g SepaFlash® Silica Flash Column, eluent of 0-20% ethyl acetate/petroleum ether gradient @ 40 mL/min) to give diethyl 2-[(3R,5R,8R,9R,10S,13S,14S,17R)-3-hydroxy-3,13-dimethyl-2,4,5,6,7,8,9,10,11,12,14,15,16,17-tetradecahydro-1H-cyclopenta[a]phenanthren-17-yl]propanedioate (1.35 g, 59% yield) as a yellow solid. LCMS (ESI) m/z, C₂₆H₄₂O₅: calculated 434.30, found (M+Na)⁺: 457.2. ¹H NMR (400 MHz, CDCl₃) δ (ppm) 4.22-4.11 (m, 4H), 3.30 (d, J=11.6 Hz, 1H), 2.26-2.16 (m, 1H), 2.00-1.88 (m, 1H), 1.87-1.77 (m, 3H), 1.65-1.61 (m, 2H), 1.49-1.02 (m, 27H), 0.71 (s, 3H).

### Preparation of diethyl 2-(cyclopropylmethyl)-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propanedioate

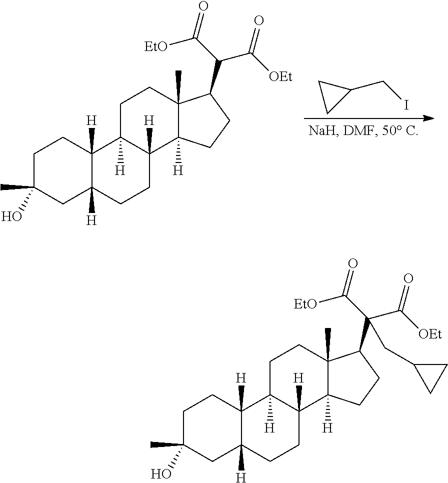

To a solution of diethyl 2-[(3R,5R,8R,9R,10S,13S,14S,17R)-3-hydroxy-3,13-dimethyl-2,4,5,6,7,8,9,10,11,12,14,15,16,17-tetradecahydro-1H-cyclopenta[a]phenanthren-17-yl]propanedioate (250 mg, 0.58 mmol, 1 eq) in DMF (5 mL) was added NaH (69 mg, 1.74 mmol, 60% in mineral oil, 3 eq) at 0° C. The resulting mixture was stirred at 0° C. for 0.5 h before iodomethylcyclopropane (209 mg, 1.15 mmol, 2 eq) was added, and the mixture was stirred at 50° C. for additional 16 h. The reaction mixture was then quenched with H₂O (5 mL) at 0° C. and extracted with EtOAc (5 mL×3). The organic layers were combined, washed with brine (15 mL), dried over Na₂SO₄, filtered, and concentrated. The residue was purified by flash silica gel chromatography (ISCO®; 4 g SepaFlash® Silica Flash Column, eluted with 0-25% ethyl acetate/petroleum ether gradient @ 20 mL/min) to give diethyl 2-(cyclopropylmethyl)-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propanedioate (170 mg, 60% yield) as a light yellow oil.

### Preparation of 2-(cyclopropylmethyl)-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propane-1,3-diol

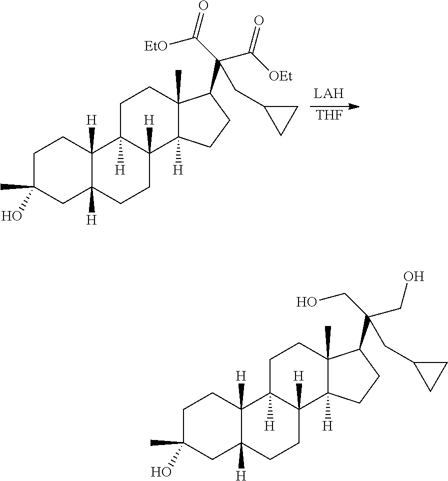

To a solution of diethyl 2-(cyclopropylmethyl)-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propanedioate (170 mg, 0.35 mmol, 1 eq) in THF (10 mL) was added LiAlH₄ (66 mg, 1.75 mmol, 5 eq) at 0° C. The resulting mixture was stirred at 20° C. for 16 h. The reaction mixture was quenched by aqueous NaOH (1.0 M, 3 mL) and extracted with EtOAc (5 mL×3). The organic layers were combined, washed with brine (15 mL), dried over Na₂SO₄, filtered, and concentrated. The residue was purified by flash silica gel chromatography (ISCO®; 4 g SepaFlash® Silica Flash Column, eluted with 25-75% ethyl acetate/petroleum ether gradient @ 20 mL/min) to give 2-(cyclopropylmethyl)-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propane-1,3-diol (120 mg, 85% yield) as a white solid.

### Preparation of (3R,5R,8R,9R,10S,13S,14S,17S)-17-[3-(cyclopropylmethyl)oxetan-3-yl]-3,13-dimethyl-2,4,5,6,7,8,9,10,11,12,14,15,16,17-tetradecahydro-1H-cyclopenta[a]phenanthren-3-ol

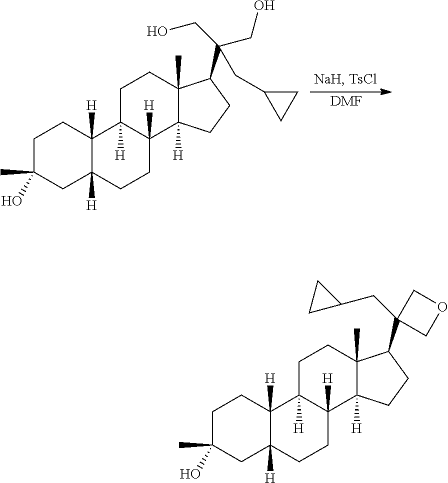

To a solution of 2-(cyclopropylmethyl)-2-[(3R,5R,8R,9R,10S,13S,14S,17S) hydroxy-3,13-dimethyl-2,4,5,6,7,8,9,10,11,12,14,15,16,17-tetradecahydro-1H-cyclopenta[a]phenanthren-17-yl]propane-1,3-diol (70 mg, 0.17 mmol, 1 eq) in DMF (3 mL) was added NaH (27.7 mg, 0.69 mmol, 60% in mineral oil, 4 eq). The resulting mixture was stirred at 20° C. for 0.5 h before TsCl (42.3 mg, 0.22 mmol, 1.3 eq) was added, and then the mixture was stirred at 20° C. for additional 16 h. The reaction mixture was quenched by H₂O (5 mL) at 0° C. and extracted with EtOAc (5 mL×3). The organic layers were combined, washed with brine (15 mL), dried over Na₂SO₄, filtered, and concentrated. The resulting residue was purified by flash silica gel chromatography (ISCO®; 4 g SepaFlash® Silica Flash Column, eluted with 0-20% ethyl acetate/petroleum ether gradient @ 20 mL/min) to give (3R,5R,8R,9R,10S,13S,14S,17S)-17-[3-(cyclopropylmethyl)oxetan-3-yl]-3,13-dimethyl-2,4,5,6,7,8,9,10,11,12,14,15,16,17-tetradecahydro-1H-cyclopenta[a]phenanthren-3-ol (19.7 mg, 29% yield) as a white solid. LCMS (ESI) m/z, C₂₆H₄₂O₂: calculated 386.32, found (M-OH)⁺: 369.2. ¹H NMR (400 MHz, CDCl₃) δ (ppm) 4.88 (d, J=6.02 Hz, 1H), 4.61-4.56 (m, 1H), 4.55-4.51 (m, 1H), 4.35 (d, J=6.5 Hz, 1H), 2.15-2.03 (m, 1H), 1.99-1.89 (m, 2H), 1.87-1.78 (m, 4H), 1.73-1.55 (m, 6H), 1.50-1.34 (m, 6H), 1.29-1.19 (m, 8H), 1.12-0.99 (m, 3H), 0.96-0.88 (m, 1H), 0.60-0.50 (m, 5H), 0.26-0.17 (m, 1H), 0.12-0.03 (m, 1H). ¹³C NMR (100 MHz, CDCl₃) δ (ppm) 75.9, 74.3, 68.0, 51.7, 48.6, 42.3, 39.7, 38.6, 37.2, 36.3, 36.1, 33.6, 30.7, 30.5, 27.4, 22.5, 22.0, 21.6, 21.4, 20.6, 20.2, 8.4, 2.4, 1.4.

### Example 10: (3R,5R,8R,9R,10S,13S,14S,17S)-17-(3-fluorooxetan-3-yl)-3,13-dimethyl-2,4,5,6,7,8,9,10,11,12,14,15,16,17-tetradecahydro-1H-cyclopenta[a]phenanthren-3-ol

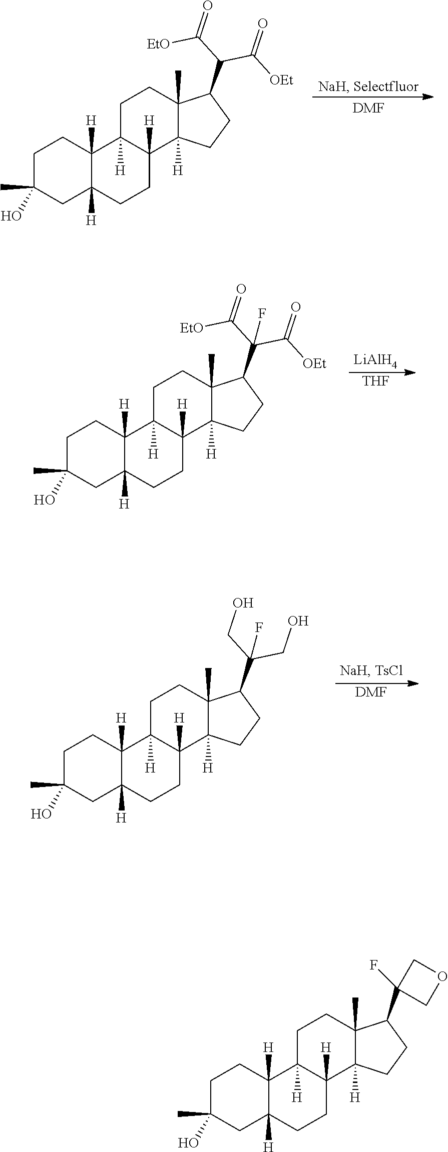

### Preparation of diethyl 2-fluoro-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propanedioate

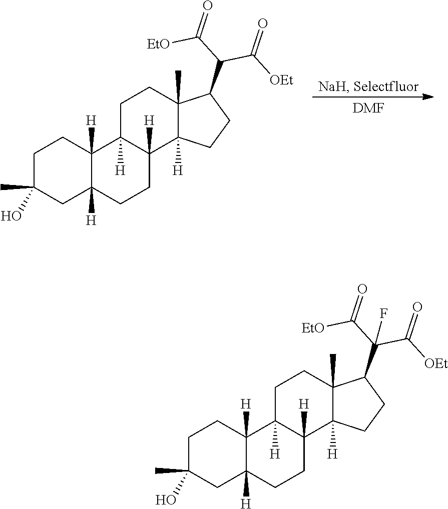

To a solution of diethyl 2-[(3R,5R,8R,9R,10S,13S,14S)-3-hydroxy-3,13-dimethyl-2,4,5,6,7,8,9,10,11,12,14,15,16,17-tetradecahydro-1H-cyclopenta[a]phenanthren-17-yl]propanedioate (500 mg, 1.15 mmol, 1 eq) in DMF (20 mL) was added NaH (230 mg, 5.75 mmol, 60% in mineral oil, 5 eq) at 0° C. The mixture was stirred at 0° C. for 0.5 h before 1-(chloromethyl)-4-fluoro-1,4-diazoniabicyclo[2.2.2]octane ditetrafluoroborate (1.22 g, 3.45 mmol, 3 eq) was added at 0° C. The resulting mixture was stirred at room temperature for 16 h. The reaction mixture was quenched by saturated aqueous NH₄Cl (20 mL) and extracted with EtOAc (15 mL×2). The organic layers were combined, washed with brine (15 mL), dried over Na₂SO₄, filtered, and concentrated. The residue was purified by flash silica gel chromatography (ISCO®; 12 g SepaFlash® Silica Flash Column, eluent of 0-10% ethyl acetate/dichloromethane gradient @ 20 mL/min) to give diethyl 2-fluoro-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propanedioate (470 mg, 90% yield) as a white solid. ¹H NMR (400 MHz, CDCl₃) δ (ppm) 4.38-4.17 (m, 4H), 2.56-2.38 (m, 1H), 1.87-1.75 (m, 5H), 1.66-1.55 (m, 7H), 1.44-1.37 (m, 4H), 1.34-1.25 (m, 12H), 1.20-1.12 (m, 3H), 1.10-1.03 (m, 2H), 0.81 (d, J=4.8 Hz, 3H). ¹⁹F NMR (376 MHz, CDCl₃) δ (ppm) −174.29.

### Preparation of 2-fluoro-2-[(3R,5R,8R,9R,10S,13S,14S,175)-3-hydroxy-3,13-dimethyl-2,4,5,6,7,8,9,10,11,12,14,15,16,17-tetradecahydro-1H-cyclopenta[a]phenanthren-17-yl]propane-1,3-diol

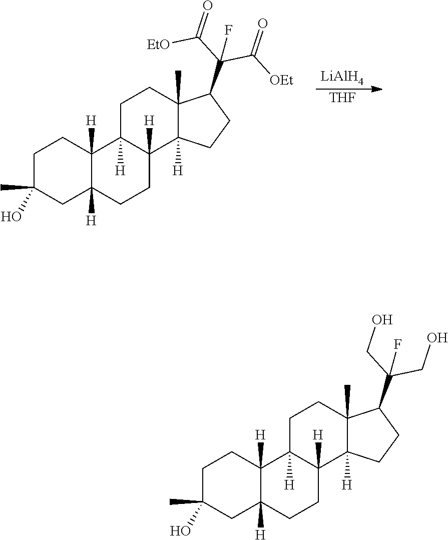

To a solution of LiAlH₄ (394 mg, 10.4 mmol, 10 eq) in THF (50 mL) was added diethyl 2-fluoro-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren yl]propanedioate (470 mg, 1.04 mmol, 1 eq). The mixture was stirred at room temperature for 4 h. The reaction mixture was quenched by H₂O (10 mL) and extracted with EtOAc (15 mL×2). The organic layers were combined, washed with brine (10 mL), dried over Na₂SO₄, filtered, and concentrated. The residue was purified by flash silica gel chromatography (ISCO®; 12 g SepaFlash® Silica Flash Column, eluent of 0-10% methanol/dichloromethane gradient @ 20 mL/min) to give 2-fluoro-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propane-1,3-diol (190 mg, 49% yield) as a white solid.

### Preparation of (3R,5R,8R,9R,10S,13S,14S,17S)-17-(3-fluorooxetan-3-yl)-3,13-dimethyl-2,4,5,6,7,8,9,10,11,12,14,15,16,17-tetradecahydro-1H-cyclopenta[a]phenanthren-3-ol

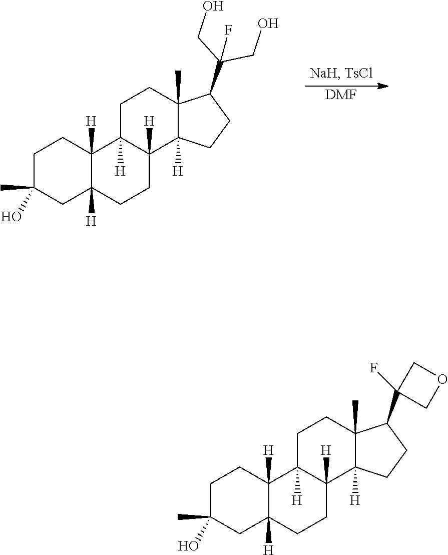

To a solution of 2-fluoro-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propane-1,3-diol (190 mg, 0.52 mmol, 1 eq) in DMF (8 mL) was added NaH (103 mg, 2.58 mmol, 60% in mineral oil, 5 eq) at 0° C. The mixture was stirred at 0° C. for 1 h before 4-methylbenzenesulfonyl chloride (118 mg, 0.62 mmol, 1.2 eq) was added at 0° C., and the resulting mixture was stirred at room temperature for another 15 h. The reaction mixture was quenched by saturated aqueous NH₄Cl (20 mL) and extracted with EtOAc (20 mL×2). The organic layers were combined, washed with brine (20 mL), dried over Na₂SO₄, filtered, and concentrated. The residue was purified by flash silica gel chromatography (ISCO®; 4 g SepaFlash® Silica Flash Column, eluent of 0-30% ethyl acetate/petroleum ether gradient @ 20 mL/min) to give (3R,5R,8R,9R,10S,13S,14S,17S)-17-(3-fluorooxetan-3-yl)-3,13-dimethyl-2,4,5,6,7,8,9,10,11,12,14,15,16,17-tetradecahydro-1H-cyclopenta[a]phenanthren-3-ol (65 mg, 36% yield) as a white solid. LCMS (ESI) m/z, C₂₂H₃₅FO₂: calculated 350.26, found [M-OH]⁺: 333.26. ¹H NMR (400 MHz, CDCl₃) δ (ppm) 4.89-4.60 (m, 4H), 2.00-1.79 (m, 7H), 1.74-1.63 (m, 2H), 1.48-1.36 (m, 5H), 1.33-0.98 (m, 14H), 0.65 (d, J=1.2 Hz, 3H). ¹⁹F NMR (376 MHz, CDCl₃) δ (ppm) −147.27. ¹³C NMR (100 MHz, CDCl₃) δ (ppm) 99.89, 97.85, 81.32, 81.07, 80.20, 79.94, 72.04, 54.88, 53.66, 43.58, 41.17, 40.35, 39.28, 37.73, 34.74, 31.39, 26.47, 26.06, 25.42, 24.25, 22.87, 12.87.

### Example 11: (3R,5R,8R,9R,10S,13S,14S,17S)-17-[3-(hydroxymethyl)oxetan-3-yl]-3,13-dimethyl-2,4,5,6,7,8,9,10,11,12,14,15,16,17-tetradecahydro-1H-cyclopenta[a]phenanthren-3-ol

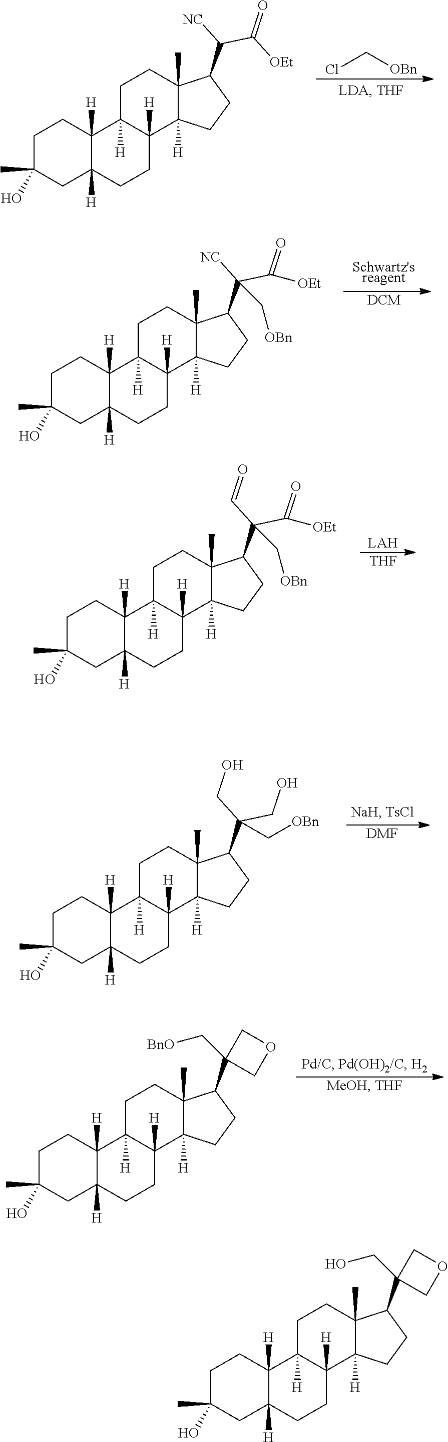

### Preparation of ethyl 3-benzyloxy-2-cyano-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propanoate

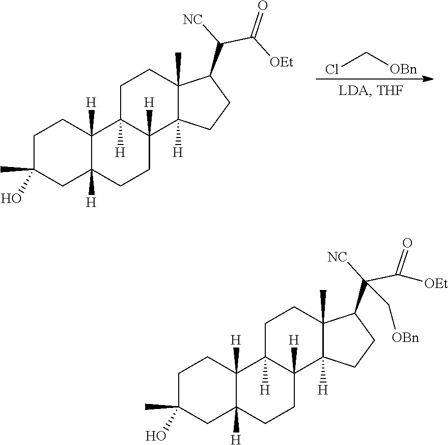

To a solution of ethyl 2-cyano-2-[(3R,5R,8R,9R,10S,13S,14S,17R)-3-hydroxy-3,13-dimethyl-2,4,5,6,7,8,9,10,11,12,14,15,16,17-tetradecahydro-1H-cyclopenta[a]phenanthren-17-yl]acetate (4.00 g, 10.3 mmol, 1 eq) in THF (50 mL) was added LDA (2.5 M in THF, 10.3 mL, 2.5 eq) at −78° C. under Na atmosphere. The mixture was stirred at −78° C. for 0.5 h before chloromethoxymethylbenzene (2.40 g, 15.4 mmol, 2.1 mL, 1.5 eq) was added at −78° C. The resulting mixture was warmed to 20° C. and stirred for another 16 h. The reaction mixture was quenched by saturated aqueous NH₄C1 (15 mL) and extracted with EtOAc (50 mL×2). The organic layers were combined, washed with brine (40 mL), dried over Na₂SO₄, filtered, and concentrated. The residue was purified by flash silica gel chromatography (ISCO®; 24 g SepaFlash® Silica Flash Column, eluted 0-20% ethyl acetate/petroleum ether gradient @ 20 mL/min) to give ethyl 3-benzyloxy-2-cyano-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren yl]propanoate (3.50 g, 66% yield) as colorless oil.

### Preparation of ethyl 2-(benzyloxymethyl)-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]-3-oxo-propanoate

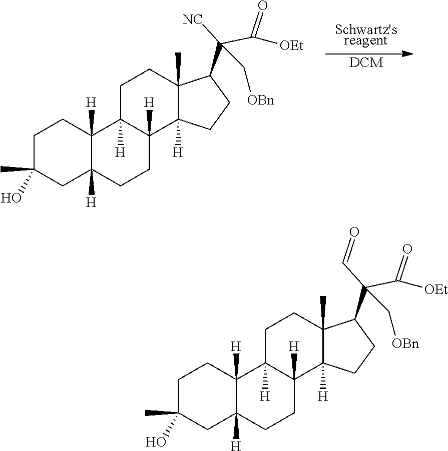

To a solution of ethyl 3-benzyloxy-2-cyano-2-(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propanoate (2.00 g, 3.94 mmol, 1 eq) in DCM (30 mL) was added bis(cyclopentadienyl)zirconium chloride hydride (4.26 g, 95% purity, 15.7 mmol, 4 eq). The resulting mixture was stirred under N₂ at 20° C. for 16 h. The reaction mixture was diluted with water (30 mL) and extracted with EtOAc (50 mL×3). The organic layers were combined, washed with brine (50 mL), dried over Na₂SO₄, filtered, and concentrated. The residue was purified by flash silica gel chromatography (ISCO®; 20 g SepaFlash® Silica Flash Column, eluted with 0-20% ethyl acetate/petroleum ether gradient @ 30 mL/min) to give ethyl 2-(benzyloxymethyl)-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]-3-oxo-propanoate (1.70 g, 84% yield) as a white solid. ¹H NMR (400 MHz, CDCl₃) δ (ppm) 10.44 (s, 1H), 7.34-7.22 (m, 5H), 4.51-4.48 (d, J=12.4 Hz, 1H), 4.37-4.34 (d, J=12.4 Hz, 1H), 4.30-4.21 (m, 2H), 4.00-3.98 (d, J=8.0 Hz, 1H), 3.65-3.63 (d, J=8.0 Hz, 1H), 2.04-1.77 (m, 8H), 1.43-0.96 (m, 22H), 0.63 (s, 3H).

### Preparation of 2-(benzyloxymethyl)-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propane-1,3-diol

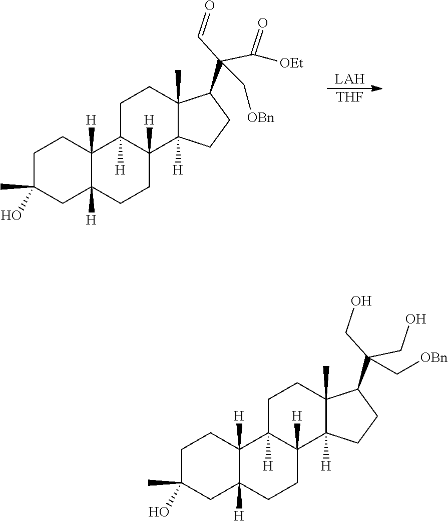

To a solution of ethyl 2-(benzyloxymethyl) [(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren yl]-3-oxo-propanoate (1.70 g, 3.33 mmol, 1 eq) in THF (40 mL) was added LiAlH₄ (379 mg, 9.99 mmol, 3 eq). The resulting mixture was stirred at 20° C. for 2 h and then quenched with 10% NaOH aqueous solution (8 mL) and extracted with EtOAc (15 mL×3). The organic layers were combined, washed with brine (10 mL), dried over Na₂SO₄, filtered, and concentrated. The resulting residue was purified by flash silica gel chromatography (ISCO®; 12 g SepaFlash® Silica Flash Column, eluted with 0-70% ethyl acetate/petroleum ether gradient @ 20 mL/min) to give 2-(benzyloxymethyl)-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propane-1,3-diol (1.30 g, 83% yield) as a colorless oil.

### Preparation of (3R,5R,8R,9R,10S,13S,14S,17S)-17-[3-(benzyloxymethyl)oxetan-3-yl]-3,13-dimethyl-2,4,5,6,7,8,9,10,11,12,14,15,16,17-tetradecahydro-1H-cyclopenta[a]phenanthren-3-ol

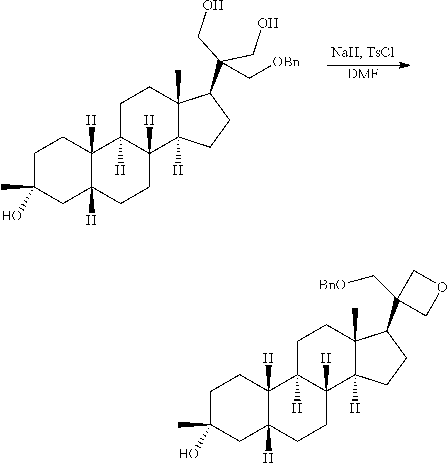

To a solution of 2-(benzyloxymethyl)-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propane-1,3-diol (1.20 g, 2.55 mmol, 1 eq) in DMF (6 mL) was added NaH (204 mg, 5.10 mmol, 60% in mineral oil, 2 eq). The resulting mixture was stirred at 20° C. for 1 h before 4-methylbenzenesulfonyl chloride (534 mg, 2.80 mmol, 1.1 eq) was added, and then the mixture was stirred at 20° C. for 2 h. The reaction mixture was quenched by water (10 mL) and extracted with EtOAc (15 mL×3). The organic layers were combined, washed with brine (10 mL), dried over Na₂SO₄, filtered, and concentrated. The residue was purified by flash silica gel chromatography (ISCO®; 12 g SepaFlash® Silica Flash Column, eluted with 0-40% ethyl acetate/petroleum ether gradient @ 20 mL/min) to give (3R,5R,8R,9R,10S,13S, 14S,17S)-17-[3-(benzyloxymethyl)oxetan-3-yl]-3,13-dimethyl-2,4,5,6,7,8,9,10,11,12,14,15,16,17-tetradecahydro-1H-cyclopenta[a]phenanthren-3-ol (650 mg, 56% yield) as white solid. ¹H NMR (400 MHz, CDCl₃) δ (ppm) 7.37-7.30 (m, 4H), 4.85-4.83 (d, J=6.4 Hz, 1H), 4.62-4.59 (d, J=12.0 Hz, 1H), 4.55-4.52 (m, 2H), 4.46-4.44 (d, J=5.6 Hz, 1H), 4.24-4.23 (d, J=6.4 Hz, 1H), 3.91-3.89 (d, J=8.8 Hz, 1H), 3.68-3.66 (d, J=8.8 Hz, 1H), 2.12-2.09 (m, 1H), 1.84-1.79 (m, 7H), 1.44-1.00 (m, 20H), 0.52 (s, 3H).

### Preparation of (3R,5R,8R,9R,10S,13S,14S,17S)-17-[3-(hydroxymethyl)oxetan-3-yl]-3,13-dimethyl-2,4,5,6,7,8,9,10,11,12,14,15,16,17-tetradecahydro-1H-cyclopenta[a]phenanthren-3-ol

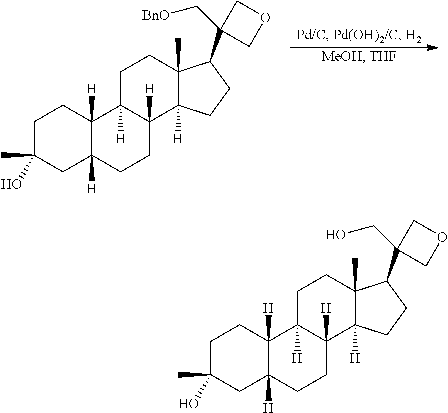

To a solution of (3R,5R,8R,9R,10S,13S,14S,17S)-17-[3-(benzyloxymethyl)oxetan-3-yl]-3,13-dimethyl-2,4,5,6,7,8,9,10,11,12,14,15,16,17-tetradecahydro-1H-cyclopenta[a]phenanthren-3-ol (650 mg, 1.44 mmol, 1 eq) in MeOH (2 mL) and THF (4 mL) was added Pd/C (50 mg, 10 wt % loading) and Pd(OH)₂/C (50 mg, 20 wt % loading). The mixture was stirred under H₂ (15 Psi) at 20° C. for 16 h. The resulting reaction mixture was filtered, and the filtrate was concentrated. The residue was purified by flash silica gel chromatography (ISCO®; 4 g SepaFlash® Silica Flash Column, eluted with 0-80% ethyl acetate/petroleum ether gradient @ 20 mL/min) to give (3R,5R,8R,9R,10S,13S,14S,17S)-17-[3-(hydroxymethyl)oxetan-3-yl]-3,13-dimethyl-2,4,5,6,7,8,9,10,11,12,14,15,16,17-tetradecahydro-1H-cyclopenta[a]phenanthren-3-ol (360 mg, 69% yield) as a white solid. LCMS (ESI) m/z, C₂₃H₃₈O₃: calculated 362.28, found [M-OH]⁺: 345.3. ¹H NMR (400 MHz, CDCl₃) δ (ppm) 4.87-4.86 (d, J=6.8 Hz, 1H), 4.56-4.55 (d, J=6.0 Hz, 1H), 4.47-4.46 (d, J=5.6 Hz, 1H), 4.25-4.24 (d, J=6.4 Hz, 1H), 4.11-4.10 (m, 1H), 3.86-3.85 (m, 1H), 2.14-1.68 (m, 10H), 1.40-1.03 (m, 17H), 0.54 (s, 3H). ¹³C NMR (100 MHz, CDCl₃) δ (ppm) 78.74, 74.85, 72.03, 67.07, 55.47, 49.50, 47.09, 43.25, 41.16, 41.13, 40.29, 39.51, 37.53, 34.65, 34.47, 31.37, 26.42, 25.93, 25.52, 25.36, 24.18, 24.09, 12.31.

### Example 12: 3-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]oxetane-3-carboxylic acid

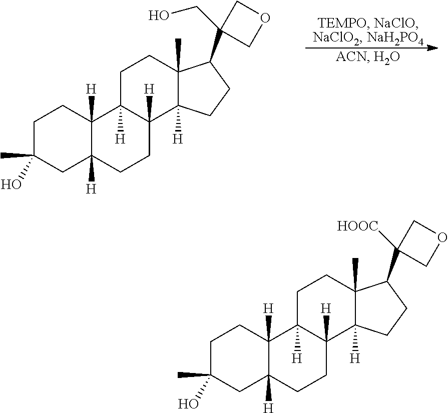

### Preparation of 3-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]oxetane-3-carboxylic acid

To a solution of (3R,5R,8R,9R,10S,13S,14S,17S)-17-[3-(hydroxymethyl)oxetan-3-yl]-3,13-dimethyl-2,4,5,6,7,8,9,10,11,12,14,15,16,17-tetradecahydro-1H-cyclopenta[a]phenanthren-3-ol (30 mg, 0.083 mmol, 1 eq) in MeCN (1.5 mL) and H₂O (1 mL) was added TEMPO (2.6 mg, 0.017 mmol, 0.2 eq), NaClO (123 mg, 0.17 mmol, 10% in H₂O, 2 eq), sodium chlorite (35 mg, 0.33 mmol, 85%, 4 eq) and sodium dihydrogen phosphate dihydrate (51 mg, 0.33 mmol, 4 eq). The resulting mixture was stirred at 50° C. for 16 h, and then was concentrated. The residue was purified by flash silica gel chromatography (ISCO®; 4 g SepaFlash® Silica Flash Column, eluted with 0-5% methanol/dichloromethane gradient @ 15 mL/min) to give 3-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]oxetane-3-carboxylic acid (5.7 mg, 18% yield) as a white solid. LCMS (ESI) m/z, C₂₃H₃₆O₄: calculated 376.26, found [M-OH]⁺: 359.3. ¹H NMR (400 MHz, CDCl₃) δ (ppm) 5.05-5.03 (d, J=6.8 Hz, 1H) 4.82-4.81 (d, J=6.4 Hz, 1H) 4.70-4.64 (m, 2H) 2.06 (s, 3H) 1.80-0.85 (m, 24H) 0.54 (s, 3H). ¹³C NMR (100 MHz, CDCl₃) δ (ppm) 177.97, 79.00, 74.15, 72.82, 55.05, 52.15, 50.01, 43.27, 41.20, 41.03, 40.29, 37.62, 37.51, 34.70, 34.26, 31.36, 29.71, 26.44, 25.97, 25.39, 23.55, 11.93.

### Example 13: 2-[3-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]oxetan-3-yl]acetonitrile

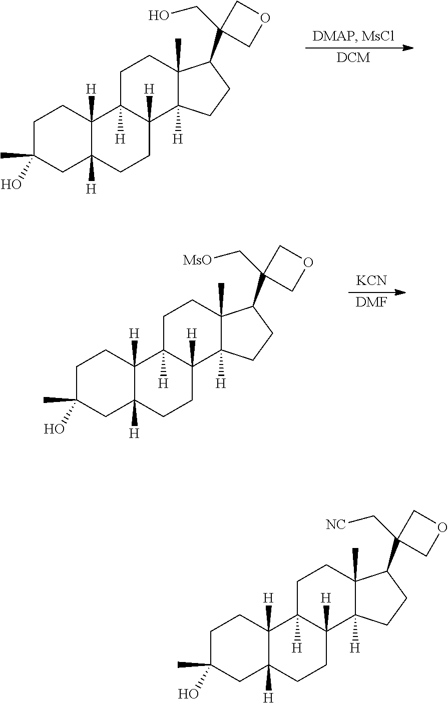

### Preparation of ethyl [3-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]oxetan-3-yl]methyl methanesulfonate

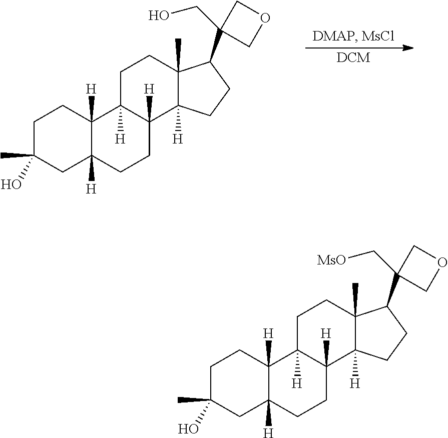

To a solution of (3R,5R,8R,9R,10S,13S,14S,17S)-17-[3-(hydroxymethyl)oxetan-3-yl]-3,13-dimethyl-2,4,5,6,7,8,9,10,11,12,14,15,16,17-tetradecahydro-1H-cyclopenta[a]phenanthren-3-ol (50 mg, 0.14 mmol, 1 eq) in DCM (2 mL) was added DMAP (50.5 mg, 0.41 mmol, 3 eq). The resulting mixture was stirred at 20° C. for 0.5 h before MsCl (18.9 mg, 0.16 mmol, 1.2 eq) was added, and then the mixture was stirred at 20° C. for another 16 h. The reaction mixture was quenched by water (2 mL) and extracted with EtOAc (5 mL×2). The organic layers were combined, washed with brine (5 mL), dried over Na₂SO₄, filtered, and concentrated. The residue was purified by flash silica gel chromatography (ISCO®; 4 g SepaFlash® Silica Flash Column, eluted with 0-35% ethyl acetate/petroleum ether gradient @ 20 mL/min) to give [3-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]oxetan-3-yl]methyl methanesulfonate (30 mg, 49% yield) as a white solid.

### Preparation of 2-[3-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]oxetan-3-yl]acetonitrile

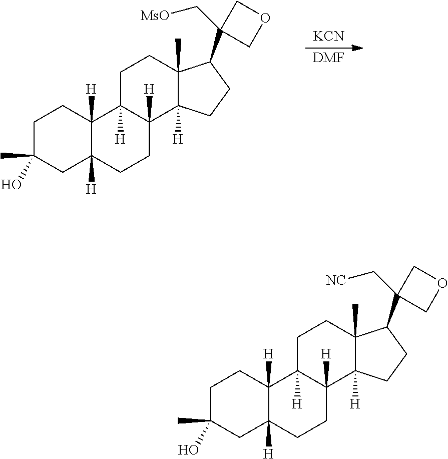

To a solution of [3-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]oxetan-3-yl]methyl methanesulfonate (30 mg, 0.068 mmol, 1 eq) in DMF (1 mL) was added KCN (8.86 mg, 0.14 mmol, 2 eq). The mixture was stirred under N₂ atmosphere at 80° C. for 16 h, then diluted with water (2 mL) and extracted with EtOAc (3 mL×3). The aqueous phase was detoxified by 10% NaClO aqueous solution and adapted to pH>11 by 1.0 M NaOH aqueous solution. The organic layers were combined, washed with brine (5 mL), dried over Na₂SO₄, filtered, and concentrated. The residue was purified by flash silica gel chromatography (ISCO®; 4 g SepaFlash® Silica Flash Column, eluted with 0-30% ethyl acetate/petroleum ether gradient @ 20 mL/min) to give 2-[3-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]oxetan-3-yl]acetonitrile (8.9 mg, 33.8% yield) as a white solid. LCMS (ESI) m/z, C₂₄H₃₇NO₂: calculated 371.28, found [M-OH]⁺: 354.28. ¹H NMR (400 MHz, CDCl₃) δ (ppm) 4.96-4.94 (d, J=6.8 Hz, 1H), 4.66-4.65 (d, J=6.4 Hz, 1H), 4.31-4.30 (d, J=6.4 Hz, 1H), 4.18-4.16 (d, J=6.8 Hz, 1H), 3.17-3.13 (d, J=16.8 Hz, 1H), 2.85-2.81 (d, J=16.8 Hz, 1H), 2.14-1.03 (m, 27H), 0.54 (s, 3H). ¹³C NMR (100 MHz, CDCl₃) δ (ppm) 117.75, 80.25, 76.48, 71.96, 55.25, 51.31, 44.15, 43.52, 41.21, 41.17, 40.30, 39.50, 37.46, 34.66, 34.60, 31.37, 27.59, 26.36, 25.89, 25.55, 25.40, 24.61, 24.01, 12.40.

### Example 14: (3R,5R,8R,9R,10S,13S,14S,17S)-17-[3-(fluoromethyl)oxetan-3-yl]-3,13-dimethyl-2,4,5,6,7,8,9,10,11,12,14,15,16,17-tetradecahydro-1H-cyclopenta[a]phenanthren-3-ol

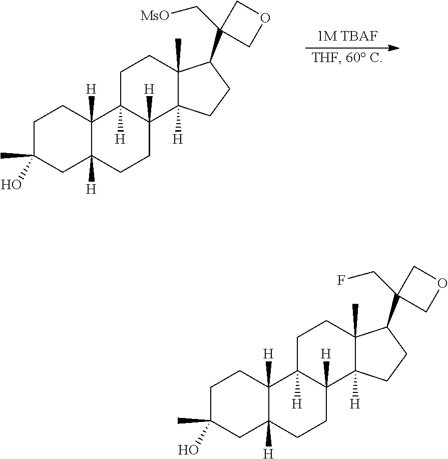

### Preparation of (3R,5R,8R,9R,10S,13S,14S,17S)-17-[3-(fluoromethyl)oxetan-3-yl]-3,13-dimethyl-2,4,5,6,7,8,9,10,11,12,14,15,16,17-tetradecahydro-1H-cyclopenta[a]phenanthren-3-ol

To a solution of [3-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]oxetan-3-yl]methyl methanesulfonate (30 mg, 0.068 mmol, 1 eq) in THF (2 mL) was added TBAF (1.0 M in THF, 0.34 mL, 5 eq) at 25° C. The resulting mixture was stirred at 60° C. for 16 h, then diluted with water (3 mL) and extracted with EtOAc (5 mL×2). The organic layers were combined, washed with brine (5 mL), dried over Na₂SO₄, filtered, and concentrated. The residue was purified by flash silica gel chromatography (ISCO®; 4 g SepaFlash® Silica Flash Column, eluted with 0-30% ethyl acetate/petroleum ether gradient @ 20 mL/min) to give (3R,5R,8R,9R,10S,13S,14S,17S)-17-[3-(fluoromethyl)oxetan-3-yl]-3,13-dimethyl-2,4,5,6,7,8,9,10,11,12,14,15,16,17-tetradecahydro-1H-cyclopenta[a]phenanthren-3-ol (8.8 mg, 35.5% yield) as white solid. LCMS (ESI) m/z, C₂₃H₃₇FO₂: calculated 364.28, found [M-OH]⁺: 347.0. ¹H NMR (400 MHz, CDCl₃) δ (ppm) 4.87-4.84 (m, 2H), 4.75-4.48 (m, 3H), 4.34-4.32 (d, J=6.8 Hz, 1H), 2.16-1.05 (m, 26H), 0.55 (s, 3H). ¹⁹F NMR (376 MHz, CDCl₃) δ (ppm) −224.86. ¹³C NMR (100 MHz, CDCl₃) δ (ppm) 87.31, 78.00, 73.72, 72.02, 55.42, 49.99, 49.96, 46.17, 43.26, 41.17, 41.14, 40.30, 39.17, 37.52, 34.66, 34.49, 31.38, 26.44, 25.93, 25.47, 24.02, 23.94, 12.42.

### Example 15: 1-[[3-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]oxetan-3-yl]methyl]pyrazole-4-carbonitrile

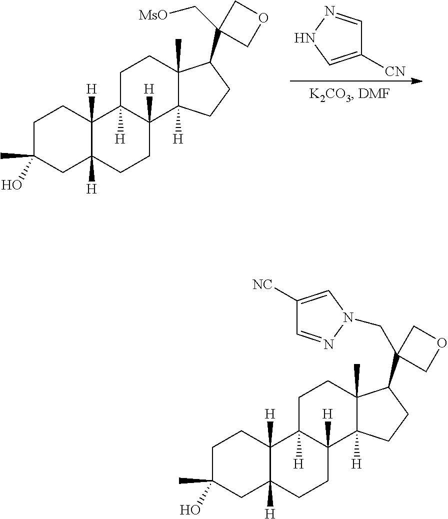

### Preparation of 1-[[3-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]oxetan-3-yl]methyl]pyrazole-4-carbonitrile

To a solution of [3-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]oxetan-3-yl]methyl methanesulfonate (10 mg, 0.023 mmol, 1 eq) and 1H-pyrazole-4-carbonitrile (2.11 mg, 0.023 mmol, 1 eq) in DMF (1 mL) was added K₂CO₃ (9.41 mg, 0.068 mmol, 3 eq). The resulting mixture was stirred at 50° C. for 16 h. and then was concentrated. The residue was purified by flash silica gel chromatography (ISCO®; 4 g SepaFlash® Silica Flash Column, eluted with 0-30% ethyl acetate/petroleum ether gradient @ 20 mL/min) to give 1-[[3-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]oxetan-3-yl]methyl]pyrazole-4-carbonitrile (3.7 mg, 37.2% yield) as a white solid. LCMS (ESI) m/z, C₂₇H₃₉N₃O₂: calculated 437.30, found (M+H)⁺: 438.3. ¹H NMR (400 MHz, CDCl₃) δ (ppm) 7.89 (s, 1H), 7.84 (s, 1H), 4.98-4.96 (d, J=6.8 Hz, 1H), 4.65-4.62 (m, 2H), 4.54-4.47 (m, 2H), 4.38 (d, J=14.0 Hz, 1H), 2.03-1.77 (m, 7H), 1.54-1.05 (m, 20H), 0.70 (s, 3H). ¹³C NMR (100 MHz, CDCl₃) δ (ppm) 142.35, 135.45, 113.33, 92.38, 78.16, 75.24, 71.98, 58.28, 55.43, 50.89, 46.66, 43.64, 41.13, 41.05, 40.24, 40.17, 37.43, 34.62, 34.57, 31.32, 26.40, 25.85, 25.47, 25.36, 24.33, 23.91, 12.90.

### Example 16 and Example 17: (3R,5R,8R,9R,10S,13S,14S,17S)-3,13-dimethyl-17-[3-(triazol-2-ylmethyl)oxetan-3-yl]-2,4,5,6,7,8,9,10,11,12,14,15,16,17-tetradecahydro-1H-cyclopenta[a]phenanthren-3-ol and (3R,5R,8R,9R,10S,13S,14S,17S)-3,13-dimethyl-17-[3-(triazol-1-ylmethyl)oxetan yl]-2,4,5,6,7,8,9,10,11,12,14,15,16,17-tetradecahydro-1H-cyclopenta[a]phenanthren-3-ol

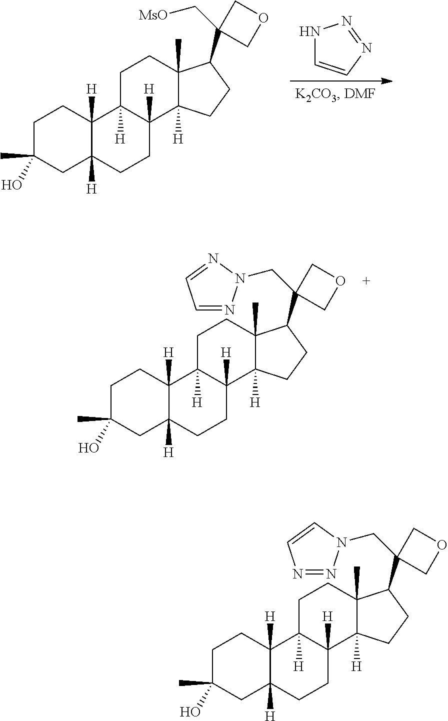

### Preparation of (3R,5R,8R,9R,10S,13S,14S,17S)-3,13-dimethyl-17-[3-(triazol-2-ylmethyl)oxetan-3-yl]-2,4,5,6,7,8,9,10,11,12,14,15,16,17-tetradecahydro-1H-cyclopenta[a]phenanthren-3-ol and (3R,5R,8R,9R,10S,13S,14S,175)-3,13-dimethyl-17-[3-(triazol-1-ylmethyl)oxetan-3-yl]-2,4,5,6,7,8,9,10,11,12,14,15,16,17-tetradecahydro-1H-cyclopenta[a]phenanthren-3-ol

To a solution of [3-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]oxetan-3-yl]methyl methanesulfonate (20 mg, 0.045 mmol, 1 eq) and 1,2,3-triazole (9.4 mg, 0.14 mmol, 3 eq) in DMF (1 mL) was added K₂CO₃ (18.8 mg, 0.14 mmol, 3 eq). The resulting mixture was stirred at 60° C. for 16 h, and then was concentrated. The residue was purified by flash silica gel chromatography (ISCO®; 4 g SepaFlash® Silica Flash Column, eluted with 0-60% ethyl acetate/petroleum ether gradient @ 20 mL/min) to give two products. (3R,5R,8R,9R,10S, 13S,14S,17S)-3,13-dimethyl-17-[3-(triazol-2-ylmethyl)oxetan-3-yl]-2,4,5,6,7,8,9,10,11,12,14,15,16,17-tetradecahydro-1H-cyclopenta[a]phenanthren-3-ol (4.0 mg, 14.2% yield) was obtained as a white solid. LCMS (ESI) m/z, C₂₅H₃₉N₃O₂: calculated 413.30, found (M+H)⁺: 414.3. ¹H NMR (400 MHz, CDCl₃) δ (ppm) 7.66 (s, 2H), 4.95-4.94 (d, J=6.8 Hz 1H), 4.87 (d, J=14.0 Hz, 1H), 4.68-4.61 (m, 4H), 1.96-1.77 (m, 7H), 1.42-1.02 (m, 20H), 0.70 (s, 3H). ¹³C NMR (100 MHz, CDCl₃) δ (ppm) 134.12, 78.35, 75.16, 72.02, 59.92, 55.47, 51.24, 46.48, 43.53, 41.15, 41.10, 40.27, 39.81, 37.47, 34.67, 34.52, 31.36, 26.46, 25.91, 25.47, 25.37, 24.25, 23.98, 12.77.

(3R,5R,8R,9R,10S,13S,14S,17S)-3,13-dimethyl-17-[3-(triazol-1-ylmethyl)oxetan-3-yl]-2,4,5,6,7,8,9,10,11,12,14,15,16,17-tetradecahydro-1H-cyclopenta[a]phenanthren-3-ol (5.6 mg, 23.8% yield) was obtained as a white solid. LCMS (ESI) m/z, C₂₅H₃₉N₃O₂: calculated 413.30, found (M+H)⁺: 414.3. ¹H NMR (400 MHz, CDCl₃) δ (ppm) 7.76 (s, 1H), 7.65 (s, 1H), 5.00-4.98 (d, J=6.8 Hz, 1H), 4.84 (d, J=14.0 Hz, 1H), 4.67-4.63 (m, 2H), 4.52-4.46 (m, 2H), 2.04-1.76 (m, 6H), 1.40-1.02 (m, 21H), 0.71 (s, 3H). ¹³C NMR (100 MHz, CDCl₃) δ (ppm) 133.60, 124.61, 78.15, 75.32, 71.96, 55.88, 55.39, 50.89, 46.55, 43.65, 41.14, 41.06, 40.24, 40.07, 37.41, 34.63, 34.51, 31.32, 26.38, 25.86, 25.47, 25.35, 24.37, 23.93, 12.88.

### Example 18: 3-((3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl)oxetane-3-carbonitrile

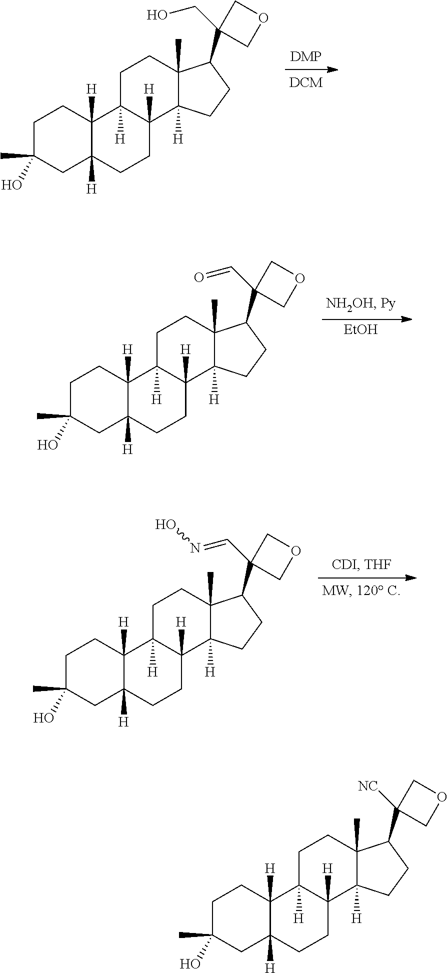

### Preparation of 3-((3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl)oxetane-3-carbaldehyde

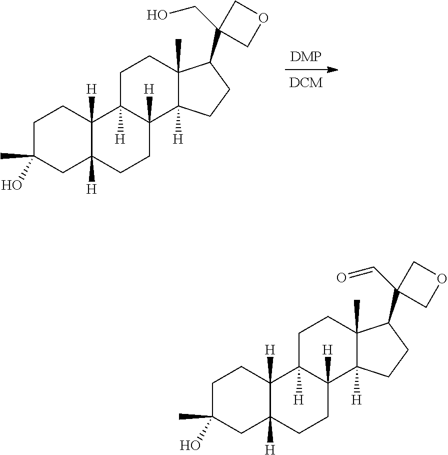

To a solution of (3R,5R,8R,9R,10S,13S,14S,17S)-17-[3-(hydroxymethyl)oxetan-3-yl]-3,13-dimethyl-2,4,5,6,7,8,9,10,11,12,14,15,16,17-tetradecahydro-1H-cyclopenta[a]phenanthren-3-ol (50 mg, 0.14 mmol, 1 eq) in DCM (3 mL) was added Dess-Martin periodinane (118 mg, 0.28 mmol, 2 eq). The resulting mixture was stirred at 25° C. for 2 h, and then the mixture was diluted with saturated NaHCO₃ (15 mL) and extracted with DCM (15 mL×3). The organic layers were combined, washed with brine (20 mL), dried over Na₂SO₄, filtered, and concentrated. The residue was purified by flash silica gel chromatography (ISCO®; 4 g SepaFlash® Silica Flash Column, eluent of 0-50% ethyl acetate/petroleum ether gradient @ 20 mL/min) to give 3-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]oxetane-3-carbaldehyde (30 mg, 60.3% yield) as a white solid.

### Preparation of 3-((3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl)oxetane-3-carbaldehyde oxime

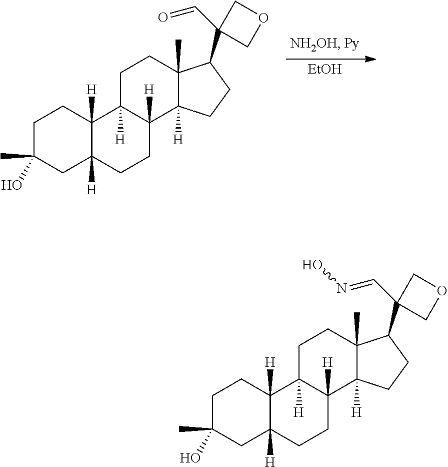

To a solution of 3-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]oxetane-3-carbaldehyde (18 mg, 0.050 mmol, 1 eq) in EtOH (1 mL) was added pyridine (39 mg, 0.50 mmol, 10 eq) and hydroxylamine HCl (6.9 mg, 0.10 mmol, 2 eq). The mixture was stirred at 25° C. for 4 h, then concentrated. The residue was diluted with EtOAc (50 mL), washed with water (25 mL) and brine (25 mL), dried over Na₂SO₄, filtered, and concentrated to give crude product 3-((3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl)oxetane-3-carbaldehyde oxime (25 mg, crude) as a yellow solid which was used directly for next step without further purification.

### Preparation of 3-((3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl)oxetane-3-carbonitrile

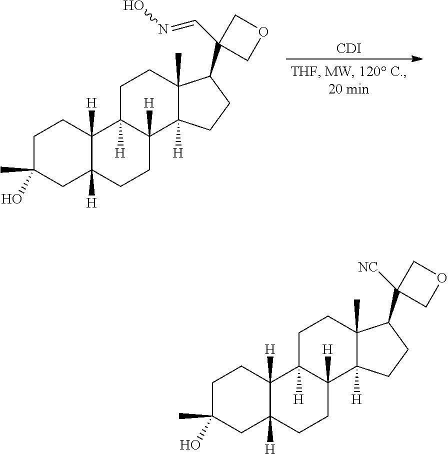

To a mixture of 3-((3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl)oxetane-3-carbaldehyde oxime (25 mg, 0.067 mmol, 1 eq) and CDI (43.2 mg, 0.27 mmol, 4 eq) in a microwave tube added THF (2 mL). The resulting mixture was microwave at 120° C. for 20 min, then concentrated. The crude product was combined with crude product from another batch and the combined crude product was purified by flash silica gel chromatography (ISCO®; 4 g SepaFlash® Silica Flash Column, eluent of 0-40% ethyl acetate/petroleum ether gradient @ 20 mL/min), the product was further purified by prep-TLC (SiO₂, petroleum ether/ethyl acetate=1/1) to give 3-[(3R,5R,8R,9R, 10S,13S,14S,17S)-3-hydroxy-3,13-dimethyl-2,4,5,6,7,8,9,10,11,12,14,15,16,17-tetradecahydro-1H-cyclopenta[a]phenanthren yl]oxetane-3-carbonitrile (4.8 mg, 20.2% yield) as a white solid. LCMS (ESI) m/z, C₂₃H₃₅NO₂: calculated 357.27, found [M-OH]⁺: 340.26. ¹H NMR (400 MHz, CDCl₃) δ (ppm) 4.90 (dd, J=8.0, 4.0 Hz, 2H), 4.72 (dd, J=12.0, 4.0 Hz, 2H), 2.10-2.05 (m, 1H), 1.95-1.87 (m, 3H), 1.82-1.74 (m, 4H), 1.71-1.62 (m, 4H), 1.47-1.39 (m, 5H), 1.36-1.26 (m, 7H), 1.20-1.01 (m, 4H), 0.76 (s, 3H). ¹³C NMR (100 MHz, CDCl₃) δ (ppm) 122.21, 78.94, 77.66, 72.03, 54.69, 53.97, 43.89, 41.10, 41.08, 40.23, 39.03, 38.56, 37.66, 34.66, 34.46, 31.29, 26.50, 25.95, 25.39, 25.25, 23.77, 23.72, 13.33.

### Example 19: (3R,5R,8R,9R,10S,13S,14S,17S)-17-[3-(difluoromethyl)oxetan-3-yl]-3,13-dimethyl-2,4,5,6,7,8,9,10,11,12,14,15,16,17-tetradecahydro-1H-cyclopenta[a]phenanthren-3-ol

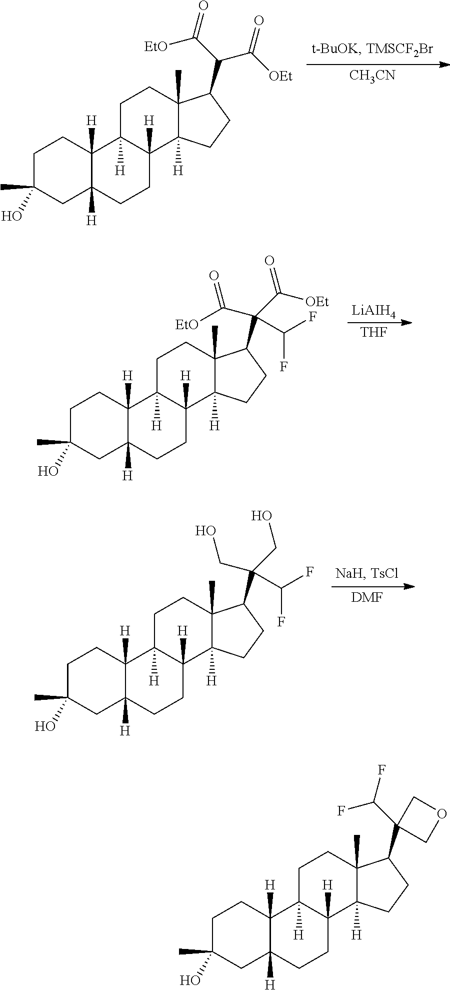

### Preparation of diethyl 2-(difluoromethyl)-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propanedioate

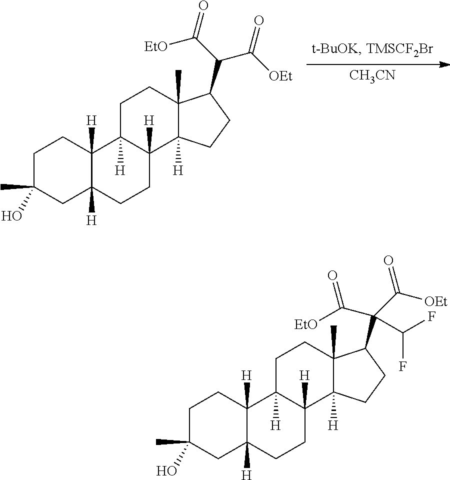

To a solution of diethyl 2-[(3R,5R,8R,9R,10S,13S,14S,17R)-3-hydroxy-3,13-dimethyl-2,4,5,6,7,8,9,10,11,12,14,15,16,17-tetradecahydro-1H-cyclopenta[a]phenanthren-17-yl]propanedioate (150 mg, 0.35 mmol, 1 eq) in CH₃CN (10 mL) was added t-BuOK (77.5 mg, 0.70 mmol, 2 eq) followed by (bromodifluoromethyl)trimethylsilane (140 mg, 0.70 mmol, 2 eq). The resulting mixture was stirred at 25° C. for additional 16 h, and then diluted with H₂O (5 mL) and extracted with EtOAc (5 mL×3). The organic layers were combined, washed with brine (15 mL), dried over Na₂SO₄, filtered, and concentrated. The resulting residue was purified by flash silica gel chromatography (ISCO®; 12 g SepaFlash® Silica Flash Column, eluted with 0-5% ethyl acetate/petroleum ether gradient @ 20 mL/min) to give diethyl 2-(difluoromethyl)-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren yl]propanedioate (110 mg, 65% yield) as a light yellow oil.

### Preparation of 2-(difluoromethyl)-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propane-1,3-diol

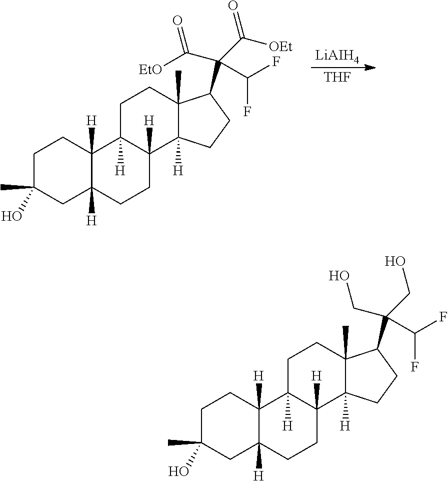

To a solution of LiAlH₄ (43.1 mg, 1.15 mmol, 5 eq) in THF (5 mL) was added diethyl 2-(difluoromethyl)-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propanedioate (110 mg, 0.23 mmol, 1 eq) in THF (3 mL) at 0° C. The resulting mixture was stirred at 25° C. for 3 h. The reaction mixture was then quenched by NaOH (1.0 M, 3 mL) and extracted with EtOAc (5 mL×3). The organic layers were combined, washed with brine (15 mL), dried over Na₂SO₄, filtered, and concentrated. The residue was purified by prep-TLC (SiO₂, petroleum ether/ethyl acetate=1/1) to give 2-(difluoromethyl)-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propane-1,3-diol (20 mg, 27% yield) as a white solid. ¹H NMR (400 MHz, CDCl₃) δ (ppm) 6.18 (t, J=56.0 Hz, 1H), 4.03-3.94 (m, 4H), 2.11-2.05 (m, 2H), 1.98-1.77 (m, 8H), 1.51-1.36 (m, 8H), 1.31-1.27 (m, 6H), 1.12-1.00 (m, 6H), 0.80 (s, 3H).

### Preparation of (3R,5R,8R,9R,10S,13S,14S,17S)-17-[3-(difluoromethyl)oxetan-3-yl]-3,13-dimethyl-2,4,5,6,7,8,9,10,11,12,14,15,16,17-tetradecahydro-1H-cyclopenta[a]phenanthren-3-ol

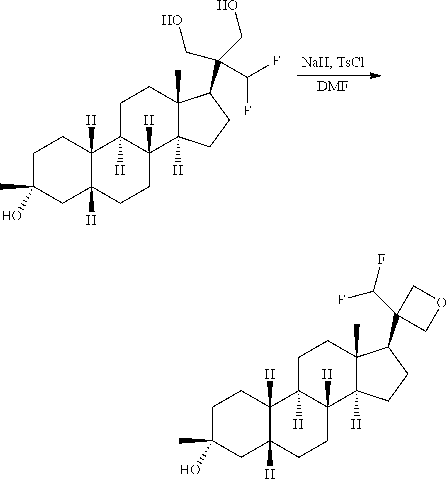

To a solution of 2-(difluoromethyl)-2-[(3R,5R,8R,9R,10S,13S,14S,17S) hydroxy-3,13-dimethyl-2,4,5,6,7,8,9,10,11,12,14,15,16,17-tetradecahydro-1H-cyclopenta[a]phenanthren-17-yl]propane-1,3-diol (20 mg, 0.050 mmol, 1 eq) in DMF (5 mL) were added NaH (7.99 mg, 0.20 mmol, 60% in mineral oil, 4 eq) and TsCl (14 mg, 0.075 mmol, 1.5 eq) at 0° C. The resulting mixture was then stirred at 25° C. for 3 h. The reaction mixture was cooled to 0° C. and added H₂O (5 mL), then extracted with EtOAc (5 mL×3). The organic layers were combined, washed with brine (10 mL), dried over Na₂SO₄, filtered, and concentrated. The resulting residue was purified by prep-TLC (SiO₂, petroleum ether/ethyl acetate=3/1) to give (3R,5R,8R,9R,10S,13S,14S,17S)-17-[3-(difluoromethyl)oxetan-3-yl]-3,13-dimethyl-2,4,5,6,7,8,9,10,11,12,14,15,16,17-tetradecahydro-1H-cyclopenta[a]phenanthren-3-ol (10.4 mg, 54% yield) as a white solid. LCMS (ESI) m/z, C₂₃H₃₆F₂O₂: calculated 382.27, found (M-OH)⁺: 365.2. ¹H NMR (400 MHz, CDCl₃) δ (ppm) 6.06 (t, J=56.0 Hz, 1H), 4.81 (d, J=6.8 Hz, 1H), 4.73 (d, J=6.4 Hz, 1H), 4.54 (d, J=7.2 Hz, 1H), 4.48 (d, J=6.0 Hz, 1H), 2.22-2.10 (m, 1H), 2.08-1.97 (m, 1H), 1.87-1.75 (m, 5H), 1.70-1.59 (m, 4H), 1.52-1.35 (m, 6H), 1.30-1.22 (m, 8H), 1.15-1.00 (m, 3H), 0.60 (s, 3H). ¹⁹F NMR (376 MHz, CDCl₃) δ (ppm) −127.99, −128.14, −128.61, −128.94, −129.28, −129.75, −129.90. ¹³C NMR (100 MHz, CDCl₃) δ (ppm) 116.36, 74.10, 72.02, 55.48, 49.00, 43.22, 41.13, 40.31, 39.25, 37.51, 34.64, 34.52, 31.38, 26.45, 25.92, 25.47, 25.38, 23.96, 12.84.

### Example 20: (3R,5R,8R,9R,10S,13S,14S,17S)-3,13-dimethyl-17-(3-(2,2,2-trifluoroethyl)oxetan-3-yl)-2,4,5,6,7,8,9,10,11,12,14,15,16,17-tetradecahydro-1H-cyclopenta[a]phenanthren-3-ol

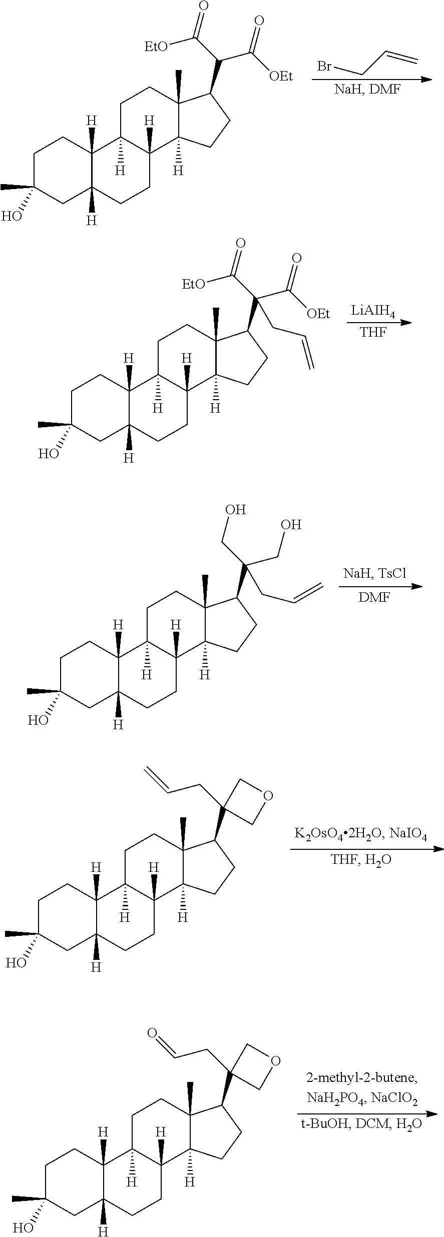

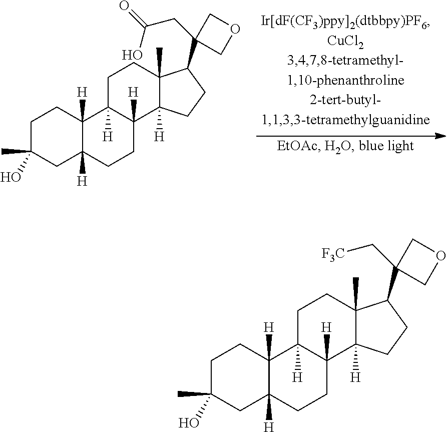

### Preparation of diethyl 2-allyl-2-((3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl)malonate

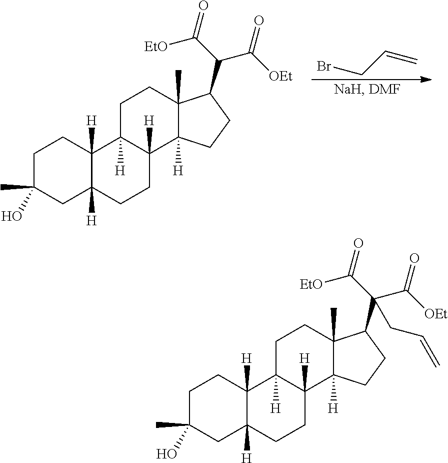

To a solution of diethyl 2-[(3R,5R,8R,9R,10S,13S,14S,17R)-3-hydroxy-3,13-dimethyl-2,4,5,6,7,8,9,10,11,12,14,15,16,17-tetradecahydro-1H-cyclopenta[a]phenanthren-17-yl]propanedioate (1.20 g, 2.76 mmol, 1 eq) in DMF (15 mL) was added NaH (552 mg, 13.8 mmol, 60% in mineral oil, 5 eq) at 0° C. The resulting mixture was stirred at 0° C. for 1 h before 3-bromoprop-1-ene (501 mg, 4.14 mmol, 1.5 eq) was added at 0° C., then the mixture was stirred at 20° C. for another 16 h. The reaction mixture was quenched by saturated aqueous NH₄Cl (30 mL) and extracted with EtOAc (30 mL×2). The organic layers were combined, washed with brine (10 mL), dried over Na₂SO₄, filtered, and concentrated. The residue was purified by flash silica gel chromatography (ISCO®; 12 g SepaFlash® Silica Flash Column, eluent of 0-20% ethyl acetate/petroleum ether gradient @ 30 mL/min) to give diethyl 2-allyl-2-((3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl)malonate (1.10 g, 83.9% yield) as a white solid. ¹H NMR (400 MHz, CDCl₃) δ (ppm) 5.90-5.73 (m, 1H), 5.09-4.95 (m, 2H), 4.23-4.04 (m, 4H), 2.99-2.88 (m, 1H), 2.63-2.52 (m, 1H), 2.28-2.08 (m, 2H), 1.94-1.76 (m, 5H), 1.67-1.34 (m, 13H), 1.28-1.23 (m, 8H), 1.21-0.90 (m, 6H), 0.67 (s, 3H).

### Preparation of 2-allyl-2-43R,5R,8R,9R,10S,13S,14S,175)-3-hydroxy-3,13-dimethyl-2,4,5,6,7,8,9,10,11,12,14,15,16,17-tetradecahydro-1H-cyclopenta[a]phenanthren-17-yl)propane-1,3-diol

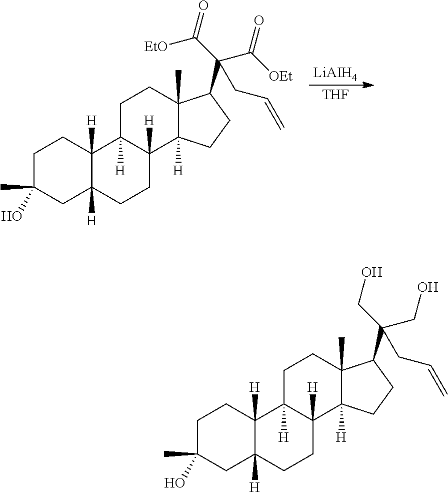

To a solution of LiAlH₄ (880 mg, 23.2 mmol, 10 eq) in THF (120 mL) was added diethyl 2-allyl-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren yl]propanedioate (1.10 g, 2.32 mmol, 1 eq) at 0° C. The mixture was stirred at 20° C. for 16 h. The reaction mixture was then quenched with NaOH aqueous solution (1.0 M, 4 mL) and H₂O (3 mL), MgSO₄ was added to the resulting mixture, the mixture was stirred at 20° C. for 0.5 h. The mixture was filtered, and the filtrate was concentrated. The residue was purified by flash silica gel chromatography (ISCO®; 12 g SepaFlash® Silica Flash Column, eluent of 0-75% ethyl acetate/petroleum ether gradient @ 25 mL/min) to give 2-allyl-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propane-1,3-diol (680 mg, 75.1% yield) as a white solid.

### Preparation of (3R,5R,8R,9R,10S,13S,14S,17S)-17-(3-allyloxetan-3-yl)-3,13-dimethyl-2,4,5,6,7,8,9,10,11,12,14,15,16,17-tetradecahydro-1H-cyclopenta[a]phenanthren-3-ol

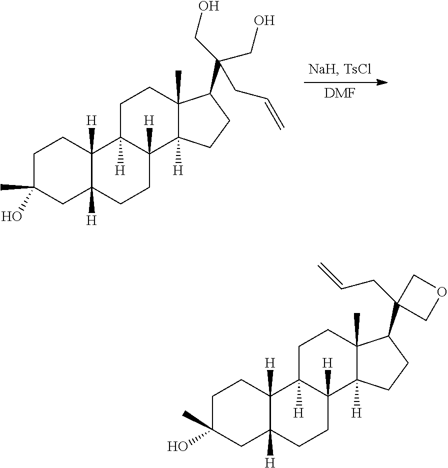

To a solution of 2-allyl-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propane-1,3-diol (680 mg, 1.74 mmol, 1 eq) in DMF (12 mL) was added NaH (278 mg, 6.96 mmol, 60% in mineral oil, 4 eq) at 0° C. The resulting mixture was stirred at 0° C. for 0.5 h before 4-methylbenzenesulfonyl chloride (398 mg, 2.09 mmol, 1.2 eq) was added at 0° C. The mixture was stirred at 25° C. for another 16 h and then added saturated aqueous NH₄Cl (20 mL) and extracted with EtOAc (30 mL×2). The organic layers were combined, washed with brine (20 mL), dried over Na₂SO₄, filtered, and concentrated. The residue was purified by flash silica gel chromatography (ISCO®; 24 g SepaFlash® Silica Flash Column, eluent of 0-30% ethyl acetate/petroleum ether gradient @ 25 mL/min) to give (3R,5R,8R,9R, 10S,13S,14S,17S)-17-(3-allyloxetan-3-yl)-3,13-dimethyl-2,4,5,6,7,8,9,10,11,12,14,15,16,17-tetradecahydro-1H-cyclopenta[a]phenanthren-3-ol (470 mg, 1.26 mmol, 72.5% yield) as a white solid. ¹H NMR (400 MHz, CDCl₃) δ (ppm) 6.03-5.86 (m, 1H), 5.24-5.12 (m, 2H), 4.90 (d, J=6.4 Hz, 1H), 4.49 (d, J=5.6 Hz, 1H), 4.31 (d, J=5.6 Hz, 1H), 4.23 (d, J=6.0 Hz, 1H), 2.76-2.63 (m, 1H), 2.57-2.46 (m, 1H), 2.14-2.01 (m, 1H), 1.93-1.57 (m, 10H), 1.47-1.02 (m, 17H), 0.57 (s, 3H).

### Preparation of 2-(3-((3R,5R,8R,9R,10S,13S,14S,17S)-3-hydroxy-3,13-dimethylhexadecahydro-1H-cyclopenta[a]phenanthren-17-yl)oxetan-3-yl)acetaldehyde

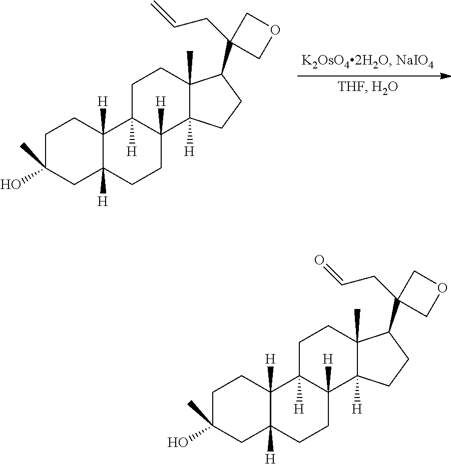

To a solution of (3R,5R,8R,9R,10S,13S,14S,17S)-17-(3-allyloxetan-3-yl)-3,13-dimethyl-2,4,5,6,7,8,9,10,11,12,14,15,16,17-tetradecahydro-1H-cyclopenta[a]phenanthren-3-ol (470 mg, 1.26 mmol, 1 eq) in THF (10 mL) and H₂O (10 mL) were added potassium osmate dihydrate (46.5 mg, 0.13 mmol, 0.1 eq) and sodium periodate (809 mg, 3.78 mmol, 0.21 mL, 3 eq). The mixture was stirred at 20° C. for 4 h and was then quenched with saturated Na₂S₂O₃ (10 mL) and extracted with EtOAc (10 mL×2). The organic layers were combined, washed with brine (10 mL), dried over Na₂SO₄, filtered, and concentrated. The residue was purified by flash silica gel chromatography (ISCO®; 12 g SepaFlash® Silica Flash Column, eluent of 0-50% ethyl acetate/petroleum ether gradient @ 25 mL/min) to give 2-[3-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]oxetan-3-yl]acetaldehyde (410 mg, 86.7% yield) as a white solid. ¹H NMR (400 MHz, CDCl₃) δ (ppm) 9.90 (s, 1H), 4.97 (d, J=6.4 Hz, 1H), 4.71 (d, J=6.4 Hz, 1H), 4.40 (d, J=6.4 Hz, 1H), 4.25 (d, J=6.4 Hz, 1H), 3.18-2.98 (m, 2H), 2.18-2.05 (m, 1H), 2.02-1.89 (m, 1H), 1.75-1.55 (m, 6H), 1.50-0.99 (m, 20H), 0.55 (s, 3H).

### Preparation of 2-(3-((3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl)oxetan-3-yl)acetic acid

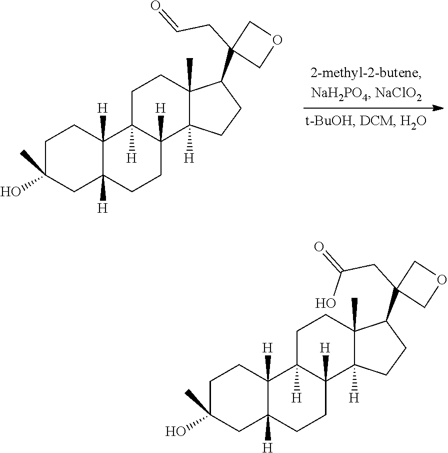

To a solution of 2-[3-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]oxetan-3-yl]acetaldehyde (71 mg, 0.19 mmol, 1 eq) in t-BuOH (2 mL), DCM (2 mL) and H₂O (1 mL) were added 2-methyl-2-butene (52.4 mg, 0.75 mmol, 4 eq), NaClO₂ (67.6 mg, 0.75 mmol, 4 eq) and NaH₂PO₄ (49.3 mg, 0.41 mmol, 2.2 eq). The resulting mixture was stirred at 25° C. for 16 h. The reaction mixture was diluted with water (15 mL) and extracted with EtOAc (15 mL×3). The organic layers were combined, washed with brine (20 mL), dried over Na₂SO₄, filtered, and concentrated. The crude product was combined with another batch and triturated with petroleum ether/EOAc (4 mL, 1/1) at 25° C. for 10 min. The mixture was filtered and the cake was dried to give 2-[3-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]oxetan-3-yl]acetic acid (51 mg, 68.5% yield) as a white solid.

### Preparation of (3R,5R,8R,9R,10S,13S,14S,175)-3,13-dimethyl-17-(3-(2,2,2-trifluoroethyl)oxetan-3-yl)-2,4,5,6,7,8,9,10,11,12,14,15,16,17-tetradecahydro-1H-cyclopenta[a]phenanthren-3-ol

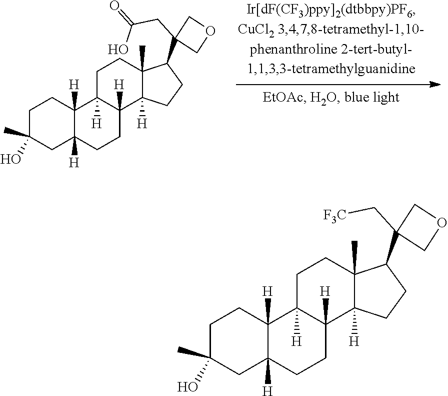

To a solution of 2-[3-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]oxetan-3-yl]acetic acid (35 mg, 0.090 mmol, 1 eq) in EtOAc (8 mL) were added 3,3-dimethyl-1-(trifluoromethyl)-1λ³,2-benziodoxole (44.4 mg, 0.13 mmol, 1.5 eq), 2-tert-butyl-1,1,3,3-tetramethylguanidine (7.8 mg, 0.045 mmol, 9.0 uL, 0.5 eq), Ir[dF(CF₃)ppy]₂(dtbbpy)PF₆ (10 mg), H₂O (48 mg, 2.69 mmol, 30 eq), 3,4,7,8-tetramethyl-1,10-phenanthroline (6.4 mg, 0.027 mmol, 0.3 eq) and CuCl₂ (2.4 mg, 0.018 mmol, 0.2 eq). The reaction was stirred and irradiated using 40 W blue LED lamps at 25° C. for 16 h. The mixture was filtered, and the filtrate was concentrated. The crude product was combined with another batch and the combined crude product was purified by prep-TLC (SiO₂, DCM/EtOAc=3/1). The product was further purified by prep-HPLC (column: Boston Prime C18 150 mm*30 mm*5 um; mobile phase: [water (0.225% FA)-ACN]; B %: 55%-85%, 9 min) to give (3R,5R,8R,9R,10S,13S,14S,17S)-3,13-dimethyl-17-[3-(2,2,2-trifluoroethyl)oxetan-3-yl]-2,4,5,6,7,8,9,10,11,12,14,15,16,17-tetradecahydro-1H-cyclopenta[a]phenanthren-3-ol (2.5 mg, 6.7% yield) as a white solid. ¹H NMR (400 MHz, CDCl₃) δ (ppm) 4.95 (d, J=8.0 Hz, 1H), 4.64 (d, J=8.0 Hz, 1H), 4.55 (d, J=8.0 Hz, 1H), 4.35 (d, J=8.0 Hz, 1H), 2.87-2.74 (m, 1H), 2.68-2.56 (m, 1H), 2.08-1.92 (m, 2H), 1.85-1.79 (m, 5H), 1.68-1.64 (m, 5H), 1.50-1.41 (m, 5H), 1.32-1.22 (m, 8H), 1.14-1.01 (m, 3H), 0.65 (s, 3H). ¹⁹F NMR (376 MHz, CDCl₃) δ (ppm) −58.68. ¹³C NMR (100 MHz, CDCl₃) δ (ppm) 125.27, 78.98, 77.93, 72.03, 55.51, 51.49, 43.87, 42.97, 41.19, 41.14, 40.79, 40.31, 39.90, 37.50, 34.70, 34.55, 31.40, 26.42, 25.94, 25.56, 25.39, 24.42, 23.97, 12.98.

### Example 21: (3R,5R,8R,9R,10S,13S,14S,17S)-17-(3-cyclopropyloxetan-3-yl)-3,13-dimethyl-2,4,5,6,7,8,9,10,11,12,14,15,16,17-tetradecahydro-1H-cyclopenta[a]phenanthren-3-ol

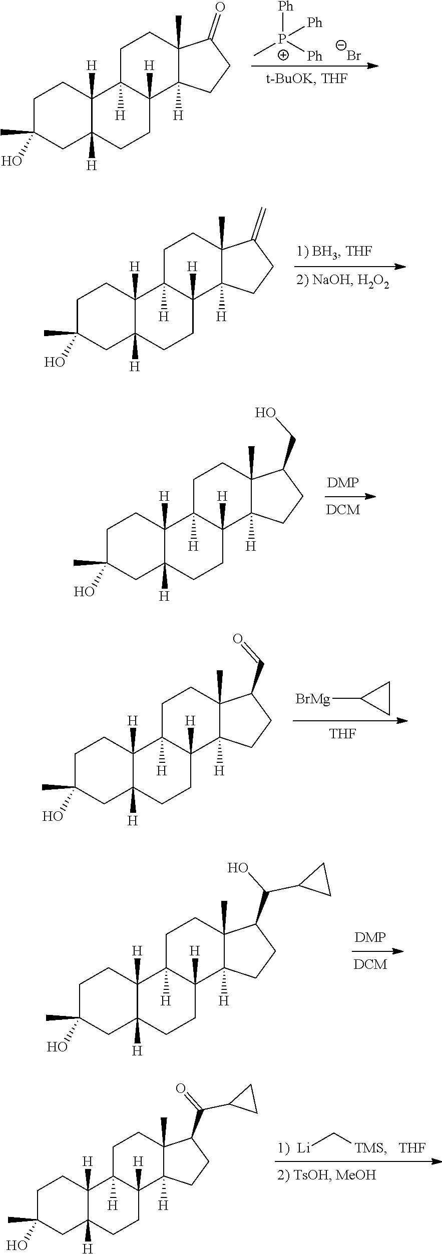

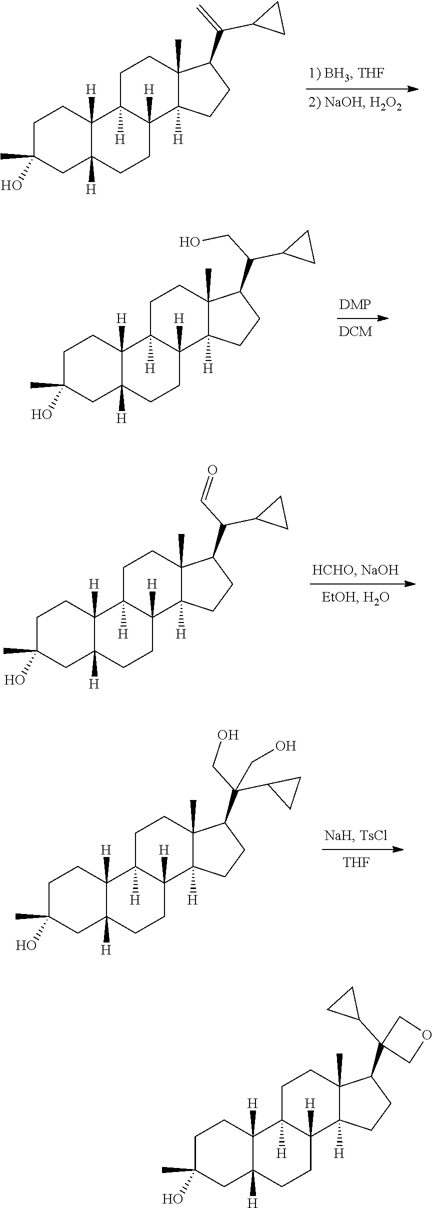

### Preparation of (3R,5R,8R,9R,10S,13S,14S)-3,13-dimethyl-17-methylene-1,2,4,5,6,7,8,9,10,11,12,14,15,16-tetradecahydrocyclopenta[a]phenanthren-3-ol

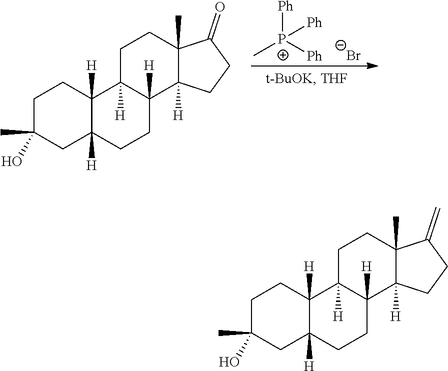

To a solution of methyltriphenylphosphonium bromide (3.69 g, 10.33 mmol, 3 eq) in THF (15 mL) was added potassium tert-butoxide (1.16 g, 10.33 mmol, 3 eq). The mixture was stirred at 60° C. for 1 h and then was added (3R,5R,8R,9R,10S,13S,14S) hydroxy-3,13-dimethyl-1,2,4,5,6,7,8,9,10,11,12,14,15,16-tetradecahydrocyclopenta[a]phenanthren-17-one (1.00 g, 3.44 mmol, 1 eq). The resulting mixture was stirred at 60° C. for another 16 h, and then diluted with EtOAc (50 mL), washed with brine (30 mL), dried over anhydrous Na₂SO₄, filtered and concentrated. The residue was purified by flash silica gel chromatography (ISCO®; 12 g SepaFlash® Silica Flash Column, eluent of 0-15% ethyl acetate/petroleum ether gradient @ 25 mL/min) to give (3R,5R,8R,9R,10S,13S,145)-3,13-dimethyl-17-methylene-1,2,4,5,6,7,8,9,10,11,12,14,15,16-tetradecahydrocyclopenta[a]phenanthren-3-ol (900 mg, 91% yield) as a white solid. ¹H NMR (400 MHz, CDCl₃) δ (ppm) 4.63-4.61 (m, 2H), 2.58-2.41 (m, 1H), 2.31-2.16 (m, 1H), 1.94-1.78 (m, 4H), 1.74-1.61 (m, 3H), 1.50-1.39 (m, 5H), 1.37-1.19 (m, 9H), 1.17-1.05 (m, 4H), 0.79 (s, 3H).

### Preparation of (3R,5R,8R,9R,10S,13S,14S,17S)-17-(hydroxymethyl)-3,13-dimethyl-2,4,5,6,7,8,9,10,11,12,14,15,16,17-tetradecahydro-1H-cyclopenta[a]phenanthren-3-ol

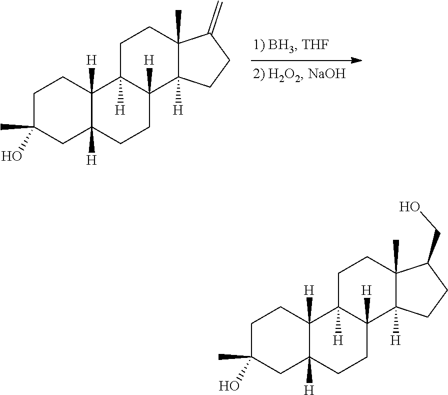

To a solution of (3R,5R,8R,9R,10S,13S,14S)-3,13-dimethyl-17-methylene-1,2,4,5,6,7,8,9,10,11,12,14,15,16-tetradecahydrocyclopenta[a]phenanthren-3-ol (900 mg, 3.12 mmol, 1 eq) in THF (20 mL) was added BH₃.THF (1.0 M in THF, 9.4 mL, 3 eq). The resulting mixture was stirred at 25° C. for 2 h before NaOH aqueous solution (2.8 M, 9.4 mL, 8.4 eq) was added slowly at 0° C., followed by adding H₂O₂ (11.0 g, 97.0 mmol, 9.4 mL, 37% in H₂O, 31 eq). The mixture was stirred at 25° C. for another 16 h and extracted with EtOAc (100 mL×2). The combined organic layer was washed with aqueous Na₂S₂O₃ (10%, 100 mL) and brine (100 mL), dried over anhydrous Na₂SO₄, filtered and concentrated. The residue was purified by flash silica gel chromatography (ISCO®; 12 g SepaFlash® Silica Flash Column, eluent of 0-40% ethyl acetate/petroleum ether gradient @ 30 mL/min) to give (3R,5R,8R,9R,10S,13S,14S,17S)-17-(hydroxymethyl)-3,13-dimethyl-2,4,5,6,7,8,9,10,11,12,14,15,16,17-tetradecahydro-1H-cyclopenta[a]phenanthren-3-ol (900 mg, 94% yield) as a white solid. ¹H NMR (400 MHz, CDCl₃) δ (ppm) 3.77-3.67 (m, 1H), 3.61-3.50 (m, 1H), 1.87-1.78 (m, 5H), 1.67-1.61 (m, 5H), 1.51-1.43 (m, 3H), 1.34-1.24 (m, 10H), 1.16-1.01 (m, 6H), 0.66 (s, 3H).

### Preparation of (3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthrene-17-carbaldehyde

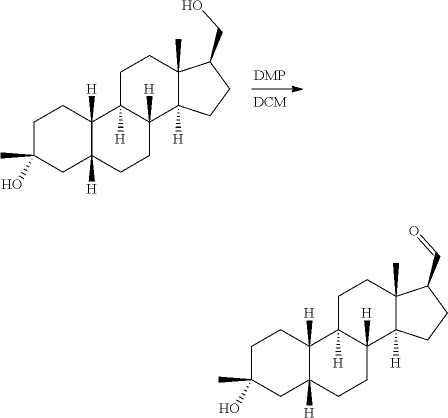

To a solution of (3R,5R,8R,9R,10S,13S,14S,17S)-17-(hydroxymethyl)-3,13-dimethyl-2,4,5,6,7,8,9,10,11,12,14,15,16,17-tetradecahydro-1H-cyclopenta[a]phenanthren-3-ol (900 mg, 2.94 mmol, 1 eq) in DCM (40 mL) was added (1,1-diacetoxy-3-oxo-1λ⁵,2-benziodoxol-1-yl) acetate (2.49 g, 5.87 mmol, 2 eq). The mixture was stirred at 25° C. for 3 h. The reaction mixture was then quenched with saturated aqueous NaHCO₃ (25 mL) and Na₂S₂O₃ (25 mL), and extracted with DCM (30 mL×2). The organic layers were combined, washed with brine (50 mL), dried over anhydrous Na₂SO₄, filtered and concentrated. The residue was purified by flash silica gel chromatography (ISCO®; 25 g SepaFlash® Silica Flash Column, eluent of 0-30% ethyl acetate/petroleum ether gradient @ 30 mL/min) to give (3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthrene-17-carbaldehyde (480 mg, 54% yield) as a colorless oil. ¹H NMR (400 MHz, CDCl₃) δ (ppm) 9.77 (d, J=2.0 Hz, 1H), 2.36-2.26 (m, 1H), 2.14-2.08 (m, 1H), 2.02-1.96 (m, 1H), 1.90-1.68 (m, 10H), 1.53-1.44 (m, 3H), 1.30-1.20 (m, 9H), 1.13-1.05 (m, 3H), 0.75 (s, 3H).

### Preparation of (3R,5R,8R,9R,10S,13S,14S,17S)-17-[cyclopropyl(hydroxy)methyl]-3,13-dimethyl-2,4,5,6,7,8,9,10,11,12,14,15,16,17-tetradecahydro-1H-cyclopenta[a]phenanthren-3-ol

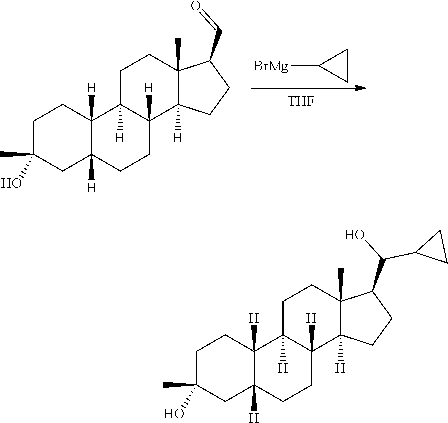

To a solution of (3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthrene-17-carbaldehyde (480 mg, 1.58 mmol, 1 eq) in THF (10 mL) was added bromo(cyclopropyl)magnesium (1.0 M in THF, 7.8 mL, 5 eq) at −78° C. The resulting mixture was stirred at 25° C. for 16 h, then quenched with saturated aqueous NH₄Cl (50 mL) and extracted with EtOAc (50 mL×2). The combined organic layers were washed with brine (50 mL), dried over anhydrous Na₂SO₄, filtered, and concentrated. The residue was purified by flash silica gel chromatography (ISCO®; 4 g SepaFlash® Silica Flash Column, eluent of 0-20% ethyl acetate/petroleum ether gradient @ 20 mL/min) to give (3R,5R,8R,9R,10S,13S,14S,17S)-17-[cyclopropyl(hydroxy)methyl]-3,13-dimethyl-2,4,5,6,7,8,9,10,11,12,14,15,16,17-tetradecahydro-1H-cyclopenta[a]phenanthren-3-ol (450 mg, 82% yield) as a white solid.

### Preparation of cyclopropyl-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]methanone

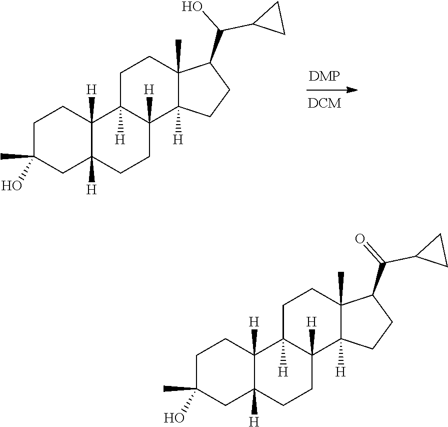

To a solution of (3R,5R,8R,9R,10S,13S,14S,17S)-17-[cyclopropyl(hydroxy)methyl]-3,13-dimethyl-2,4,5,6,7,8,9,10,11,12,14,15,16,17-tetradecahydro-1H-cyclopenta[a]phenanthren-3-ol (450 mg, 1.30 mmol, 1 eq) in DCM (25 mL) was added (1,1-diacetoxy-3-oxo-1λ⁵,2-benziodoxol-1-yl) acetate (1.10 g, 2.60 mmol, 2 eq). The resulting mixture was stirred at 25° C. for 3 h and then quenched with saturated aqueous NaHCO₃ (20 mL) and extracted with DCM (20 mL×2). The combined organic layers were washed with brine (20 mL), dried over Na₂SO₄, filtered, and concentrated. The residue was purified by flash silica gel chromatography (ISCO®; 12 g SepaFlash® Silica Flash Column, eluted with 0-20% ethyl acetate/petroleum ether gradient @ 20 mL/min) to give cyclopropyl-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]methanone (270 mg, 60% yield) as a white solid. LCMS (ESI) m/z, C₂₃H₃₆O₂: calculated 344.27, found (M+H)⁺: 345.1. ¹H NMR (400 Hz, CDCl₃) δ (ppm) 2.78 (t, J=8.8 Hz, 1H), 2.29-2.17 (m, 1H), 2.15-2.08 (m, 1H), 1.94-1.77 (m, 4H), 1.69-1.57 (m, 9H), 1.57-1.48 (m, 2H), 1.36-1.23 (m, 8H), 1.12-1.05 (m, 3H), 1.03-0.78 (m, 4H), 0.59 (s, 3H). ¹³C NMR (100 Hz, CDCl₃) δ (ppm) 211.30, 72.11, 64.58, 55.71, 44.80, 41.77, 41.14, 40.30, 39.22, 37.68, 34.73, 34.48, 31.37, 26.49, 26.08, 25.73, 25.44, 24.31, 22.20, 21.37, 13.74, 11.14, 10.70.

### Preparation of (3R,5R,8R,9R,10S,13S,14S,17S)-17-(1-cyclopropylvinyl)-3,13-dimethyl-2,4,5,6,7,8,9,10,11,12,14,15,16,17-tetradecahydro-1H-cyclopenta[a]phenanthren-3-ol

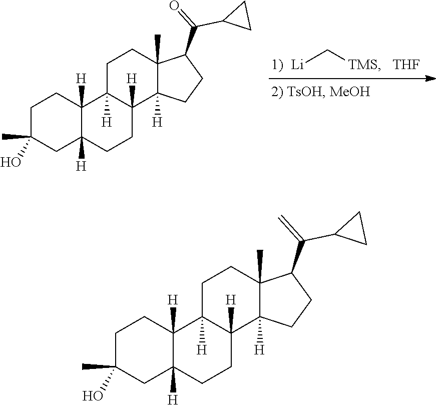

To a solution of cyclopropyl-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]methanone (140 mg, 0.41 mmol, 1 eq) in THF (10 mL) was added trimethylsilylmethyllithium (0.56 M in pentane, 7.3 mL, 10 eq) at −40° C. The mixture was warm to 25° C. and stirred at 25° C. for 16 h. The resulting mixture was then concentrated, and the residue was diluted with MeOH (5 mL). The resulting mixture was added 4-methylbenzenesulfonic acid (706 mg, 4.10 mmol, 10 eq), then was stirred at 25° C. for 1 h. The reaction mixture was diluted with saturated aqueous NaHCO₃ (10 mL) and extracted with EtOAc (10 mL×2). The combined organic layers were washed with brine (10 mL), dried over anhydrous Na₂SO₄, filtered and concentrated. The residue was purified by flash silica gel chromatography (ISCO®; 4 g SepaFlash® Silica Flash Column, eluent of 0-10% ethyl acetate/petroleum ether gradient @ 20 mL/min) to give (3R,5R,8R,9R,10S,13S,14S,17S)-17-(1-cyclopropylvinyl)-3,13-dimethyl-2,4,5,6,7,8,9,10,11,12,14,15,16,17-tetradecahydro-1H-cyclopenta[a]phenanthren-3-ol (135 mg, 96% yield) as a colorless oil. LCMS (ESI) m/z, C₂₄H₃₈O: calculated 342.29, found [M-OH]⁺: 325.2. ¹H NMR (400 MHz, CDCl₃) δ (ppm) 4.63-4.61 (m, 2H), 2.28-2.17 (m, 1H), 1.98-1.92 (m, 1H), 1.91-1.79 (m, 4H), 1.74-1.64 (m, 4H), 1.48-1.41 (m, 4H), 1.32-1.18 (m, 11H), 1.11-0.99 (m, 3H), 0.76-0.28 (m, 8H).

### Preparation of (3R,5R,8R,9R,10S,13S,14S,17S)-17-(1-cyclopropyl-2-hydroxy-ethyl)-3,13-dimethyl-2,4,5,6,7,8,9,10,11,12,14,15,16,17-tetradecahydro-1H-cyclopenta[a]phenanthren-3-ol

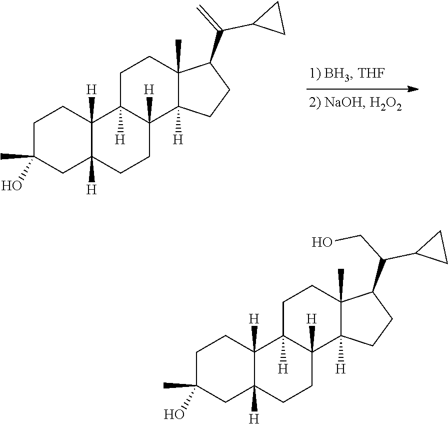

To a solution of (3R,5R,8R,9R,10S,13S,14S,17S)-17-(1-cyclopropylvinyl)-3,13-dimethyl-2,4,5,6,7,8,9,10,11,12,14,15,16,17-tetradecahydro-1H-cyclopenta[a]phenanthren-3-ol (135 mg, 0.40 mmol, 1 eq) in THF (15 mL) was added BH₃.THF (1.0 M in THF, 4.0 mL, 10 eq). The mixture was stirred at 25° C. for 2 h before NaOH aqueous solution (2.8 M, 4.0 mL, 28 eq) was added dropwise at 0° C., followed by H₂O₂ (4.65 g, 41.0 mmol, 4.0 mL, 30% in H₂O, 104 eq). The mixture was stirred at 25° C. for additional 16 h and extracted with EtOAc (20 mL×2). The combined organic layers were washed with aqueous Na₂S₂O₃ (10%, 20 mL), brine (20 mL), dried over anhydrous Na₂SO₄, filtered, and concentrated. The residue was purified by flash silica gel chromatography (ISCO®; 4 g SepaFlash® Silica Flash Column, eluted with 0-50% ethyl acetate/petroleum ether gradient @ 20 mL/min) to give (3R,5R,8R,9R,10S,13S,14S,17S)-17-(1-cyclopropylvinyl)-3,13-dimethyl-2,4,5,6,7,8,9,10,11,12,14,15,16,17-tetradecahydro-1H-cyclopenta[a]phenanthren-3-ol (140 mg, 97% yield) as a white solid.

### Preparation of 2-cyclopropyl-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]acetaldehyde

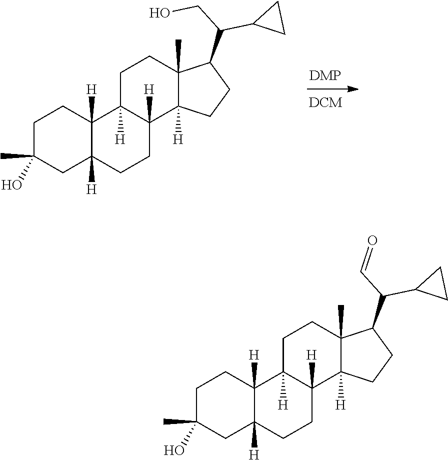

To a solution of (3R,5R,8R,9R,10S,13S,14S,17S)-17-(1-cyclopropyl-2-hydroxy-ethyl)-3,13-dimethyl-2,4,5,6,7,8,9,10,11,12,14,15,16,17-tetradecahydro-1H-cyclopenta[a]phenanthren-3-ol (140 mg, 0.39 mmol, 1 eq) in DCM (10 mL) was added (1,1-diacetoxy-3-oxo-1λ⁵,2-benziodoxol-1-yl) acetate (330 mg, 0.78 mmol, 0.24 mL, 2 eq). The mixture was stirred at 25° C. for 3 h, and then quenched with saturated NaHCO₃ (10 mL) and extracted with EtOAc (20 mL×2). The organic layers were combined, washed with brine (10 mL), dried over Na₂SO₄, filtered, and concentrated. The residue was purified by flash silica gel chromatography (ISCO®; 4 g SepaFlash® Silica Flash Column, eluent of 0-20% ethyl acetate/petroleum ether gradient @ 20 mL/min) to give 2-cyclopropyl-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]acetaldehyde (70 mg, 50% yield) as a colorless oil.

### Preparation of 2-cyclopropyl-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propane-1,3-diol

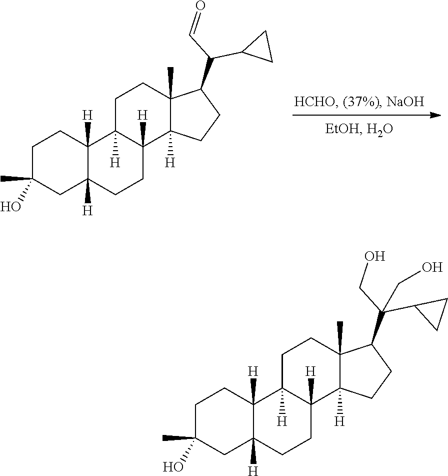

To a solution of 2-cyclopropyl-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]acetaldehyde (40 mg, 0.11 mmol, 1 eq) in EtOH (2 mL) and H₂O (1.5 mL) was added NaOH aqueous solution (1.0 M, 0.56 mL, 5 eq) and HCHO (1.09 g, 13.4 mmol, 1.33 mL, 37% in H₂O, 120 eq). The resulting mixture was stirred at 25° C. for 16 h. The reaction mixture was quenched by saturated aqueous NH₄C1 (10 mL) and extracted with EtOAc (10 mL×2). The organic layers were combined, washed with brine (10 mL), dried over Na₂SO₄, filtered, and concentrated. The residue was purified by flash silica gel chromatography (ISCO®; 4 g SepaFlash® Silica Flash Column, eluent of 0-10% methanol/dichloromethane gradient @ 20 mL/min) to give 2-cyclopropyl-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propane-1,3-diol (15 mg, 23% yield, 66% purity) as a colorless oil. LCMS (ESI) m/z, C₂₅H₄₂O₃: calculated 390.31, found [M-OH]⁺: 373.3. ¹H NMR (400 MHz, CDCl₃) δ (ppm) 3.81-3.75 (m, 1H), 3.57-3.48 (m, 3H), 2.02-1.90 (m, 3H), 1.89-1.70 (m, 7H), 1.69-1.57 (m, 4H), 1.53-1.43 (m, 2H), 1.34-1.22 (m, 8H), 1.21-0.97 (m, 6H), 0.89 (s, 3H), 0.86-0.78 (m, 1H), 0.49-0.27 (m, 4H).

### Preparation of (3R,5R,8R,9R,10S,13S,14S,17S)-17-(3-cyclopropyloxetan-3-yl)-3,13-dimethyl-2,4,5,6,7,8,9,10,11,12,14,15,16,17-tetradecahydro-1H-cyclopenta[a]phenanthren-3-ol

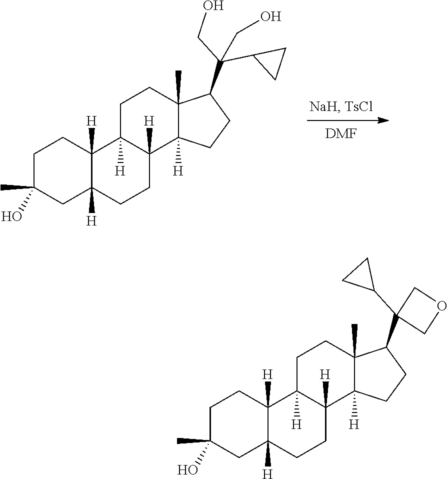

To a solution of 2-cyclopropyl-2-[(3R,5R,8R,9R,10S,13S,14S,17S) hydroxy-3,13-dimethyl-2,4,5,6,7,8,9,10,11,12,14,15,16,17-tetradecahydro-1H-cyclopenta[a]phenanthren-17-yl]propane-1,3-diol (10 mg, 0.026 mmol, 1 eq) in DMF (2 mL) was added NaH (5.2 mg, 0.13 mmol, 60% in mineral oil, 5 eq) at 0° C. The mixture was stirred at 0° C. for 0.5 h before 4-methylbenzenesulfonyl chloride (7.4 mg, 0.040 mmol, 1.5 eq) was added at 0° C. The mixture was stirred at 25° C. for 16 h, then quenched with saturated aqueous NH₄Cl (10 mL) and extracted with EtOAc (10 mL×2). The organic layers were combined, washed with brine (10 mL), dried over Na₂SO₄, filtered, and concentrated. The crude product was combined with another batch and purified by prep-TLC (SiO₂, petroleum ether/ethyl acetate=3/1) to give (3R,5R,8R,9R,10S,13S,14S,17S)-17-(3-cyclopropyloxetan-3-yl)-3,13-dimethyl-2,4,5,6,7,8,9,10,11,12,14,15,16,17-tetradecahydro-1H-cyclopenta[a]phenanthren-3-ol (5.2 mg, 19% yield). LCMS (ESI) m/z, C₂₅H₄₀O₂: calculated 372.30, found [M-OH]⁺: 355.2. ¹H NMR (400 MHz, CDCl₃) δ (ppm) 4.71 (d, J=6.0 Hz, 1H), 4.43 (d, J=6.0 Hz, 1H), 4.10-4.05 (m, 2H), 2.13-2.04 (m, 2H), 2.00-1.90 (m, 1H), 1.88-1.79 (m, 3H), 1.76-1.65 (m, 2H), 1.47-1.35 (m, 6H), 1.30-1.22 (m, 9H), 1.15-0.96 (m, 5H), 0.74-0.41 (m, 8H). ¹³C NMR (100 MHz, CDCl₃) δ (ppm) 78.99, 75.26, 72.08, 56.17, 55.54, 44.86, 43.53, 41.26, 41.19, 40.36, 39.60, 37.65, 34.74, 34.52, 31.43, 26.46, 26.00, 25.59, 25.41, 24.47, 24.08, 17.26, 12.74, 3.74, 1.63.

### Example 22: (3R,5R,8R,9R,10S,13S,14S,17S)-17-(3-ethynyloxetan-3-yl)-3,13-dimethyl-2,4,5,6,7,8,9,10,11,12,14,15,16,17-tetradecahydro-1H-cyclopenta[a]phenanthren-3-ol

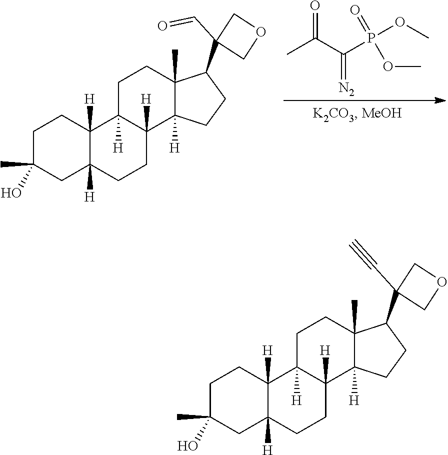

### Preparation of (3R,5R,8R,9R,10S,13S,14S,17S)-17-(3-ethynyloxetan-3-yl)-3,13-dimethyl-2,4,5,6,7,8,9,10,11,12,14,15,16,17-tetradecahydro-1H-cyclopenta[a]phenanthren-3-ol

To a solution of 3-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]oxetane-3-carbaldehyde (190 mg, 0.53 mmol, 1 eq) and K₂CO₃ (145 mg, 1.05 mmol, 2 eq) in MeOH (6 mL) was added 1-diazo-l-dimethoxyphosphoryl-propan-2-one (111 mg, 0.58 mmol, 1.1 eq) at 0° C. The resulting mixture was stirred at 20° C. for 3 h, then filtered and the filtrate was concentrated under reduced pressure. The residue was purified by flash silica gel chromatography (ISCO®; 24 g SepaFlash® Silica Flash Column, eluted with 0-30% ethyl acetate/petroleum ether gradient @ 20 mL/min) to give (3R,5R,8R,9R,10S,13S,14S,17S)-17-(3-ethynyloxetan-3-yl)-3,13-dimethyl-2,4,5,6,7,8,9,10,11,12,14,15,16,17-tetradecahydro-1H-cyclopenta[a]phenanthren-3-ol (120 mg, 63.5% yield) as a white solid. LCMS (ESI) m/z, C₂₄H₃₆O₂: calculated 356.27, found [M-OH]⁺: 339.3. ¹H NMR (400 MHz, CDCl₃) δ (ppm) 4.74-4.70 (m, 4H), 2.46 (s, 1H), 2.04-1.07 (m, 27H), 0.71 (s, 3H). ¹³C NMR (100 MHz, CDCl₃) δ (ppm) 87.53, 81.95, 81.39, 73.63, 72.04, 55.10, 55.00, 44.03, 41.19, 40.35, 39.59, 39.28, 37.74, 34.77, 34.53, 31.41, 26.46, 26.05, 25.43, 23.84, 23.69, 13.35.

### Example 23: (3R,5R,8R,9R,10S,13S,14S,17R)-17-(oxetan-3-yl)-3,13-dimethyl-2,4,5,6,7,8,9,10,11,12,14,15,16,17-tetradecahydro-1H-cyclopenta[a]phenanthren-3-ol

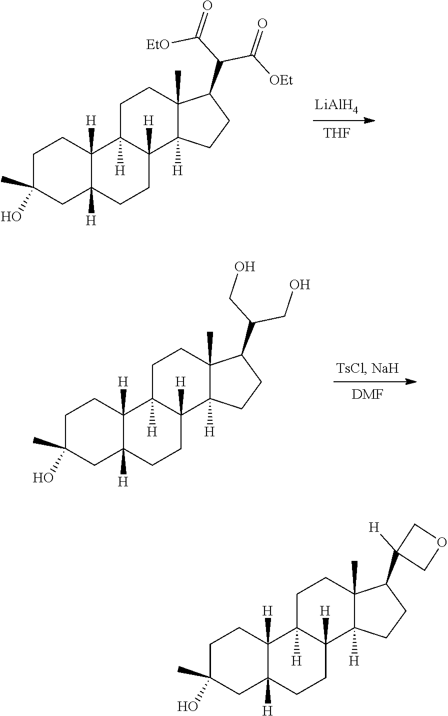

(3R,5R,8R,9R,10S,13S,14S,17R)-17-(oxetan-3-yl)-3,13-dimethyl-2,4,5,6,7,8,9,10,11,12,14,15,16,17-tetradecahydro-1H-cyclopenta[a]phenanthren-3-ol (12 mg, white solid) was prepared as described herein for the synthesis of (3R,5R,8R,9R,10S,13S,14S,17S)-17-[3-(cyclopropylmethyl)oxetan-3-yl]-3,13-dimethyl-2,4,5,6,7,8,9,10,11,12,14,15,16,17-tetradecahydro-1H-cyclopenta[a]phenanthren-3-ol, substituting diethyl 2-(cyclopropylmethyl)-2-[(3R,5R,8R,9R,10S,13S,14S,17S)-3-hydroxy-3,13-dimethyl-2,4,5,6,7,8,9,10,11,12,14,15,16,17-tetradecahydro-1H-cyclopenta[a]phenanthren-17-yl]propanedioate for diethyl 2-[(3R,5R,8R,9R,10S,13S,14S,17R)-3-hydroxy-3,13-dimethyl-2,4,5,6,7,8,9,10,11,12,14,15,16,17-tetradecahydro-1H-cyclopenta[a]phenanthren-17-yl]propanedioate. LCMS (ESI) m/z, C₂₂H₃₆O₂: calculated 332.27, found [M-OH]⁺: 315.3. ¹H NMR (400 MHz, CDCl₃) δ (ppm) 4.72-4.69 (m, 2H), 4.57-4.49 (m, 2H), 3.14-3.04 (m, 1H) 1.82-1.63 (m, 7H), 1.49-1.03 (m, 21H), 0.55 (s, 3H). ¹³C NMR (100 MHz, CDCl₃) δ (ppm) 77.24, 72.03, 54.55, 54.41, 42.83, 41.59, 41.17, 40.36, 38.76, 37.86, 36.69, 34.79, 34.52, 31.40, 26.43, 26.14, 25.47, 25.35, 25.18,

### Assay Methods

Compounds provided herein can be evaluated in various assays. A patch clamp electrophysiology assay is described here.

Cellular electrophysiology was used to measure the pharmacology properties of the GABA receptor modulators. The GABA_{A} channels are expressed in stable cell lines described herein. The parental cell line used to express the human GABA_{A} channels is the human embryonic kidney (HEK293) cell line expressing the Tetracycline repressor protein to support inducible expression of the target proteins.

For the GABA_{A} α4β3δ cell line, the α4 and β3 subunits are under control of tetracycline inducible expression system, while the δ subunit is constitutively expressed. For the GABA_{A} α1β2γ2 cell line, the α1 and β2 subunits are under control of tetracycline inducible expression system, whist the γ2 subunit is constitutively expressed.

Assessment of the compounds against GABA_{A} α1β2γ2 and GABA_{A} α4β3δ was performed using the SyncroPatch automated platform in positive allosteric modulator (PAM) mode. Six concentrations of 20 nM, 62 nM, 185 nM, 555 nM, 1667 nM and 5000 nM were tested across the plate, with a single concentration of compound per well. A minimum of 3 cells were obtained per each concentration of a compound.

Automated patch clamp-recordings were performed using the SyncroPatch 384PE. The voltage protocol generation and data collection were performed with PatchController384 V1.6.6 and Data Controller V1.6.0. A steady state voltage pulse at −80 mV was applied during the assay.

The stacked addition protocol was used, in which γ-aminobutyric acid (GABA) was rapidly applied and then washed off from the cell. To test for positive allosteric modulator activity (PAM), first the agonist GABA EC₂₀ (EC₂₀: 10 μM for GABA_{A} α4β3δ channel and 14 μM GABA for the GABA_{A} α1β2γ2 channel) was applied twice (with wash steps in-between) as a control and to show activation reproducibility, followed by a 1-2 minutes pre-incubation of the tested compound and then re-application of GABA EC₂₀ in the presence of a testing compound. Finally, following a further wash step, maximum GABA (10 mM) was applied.

An Allopregnanolone concentration response was tested as the control PAM on the compound plates. The fold increase was generated using the following equation, (I_{comp}/I_{control})−1, where I_{comp} is the current amplitude in the presence of the compound and I_{control} is the current amplitude in the presence of GABA EC₂₀ alone. This builds an EC₅₀ concentration-response curve, where 0 represents no PAM activity and >0 represents PAM activity.

A maximum % Emax for each compound is generated using the following equation, (I_{MaxComp}/I_{AveMaxAllo})*100, where I_{Maxcomp} is the individual maximum fold increase in current for each compound and I_{AveMaxAllo} is the average maximum fold increase generated in the presence of Allopregnanolone. The resultant % Eₘₐₓ values are then averaged. The compound and Allopregnanolone values were obtained from separate cells which were tested in the same compound run. Two GABA_{A} subtypes were tested against each of the compounds as well as the positive control, Allopregnanolone.

The EC₅₀ values and % Eₘₐₓ (relative to Allopregnanolone) generated are summarized in Table 2.

TABLE 2
EC₅₀ (μM) and Eₘₐₓ (relative to Allopregnanolone) of
compounds tested against GABAA receptors in PAM mode.

| | GABAA α1β2γ2 | | GABAA α4β3δ | |
|---|---|---|---|---|
| | PAM | | PAM | |
| | EC₅₀ | % Eₘₐₓ | EC₅₀ | % Eₘₐₓ |
| Example | (μM) | (to Allo) | (μM) | (to Allo) |
| allopregnanolone | A | +++ | A | +++ |
| 1 | B | + | D | + |
| 2 | C | +++ | A | +++ |
| 3 | C | + | C | + |
| 5 | B | ++ | D | + |
| 6 | A | +++ | C | +++ |
| 7 | B | +++ | B | +++ |
| 9 | A | ++ | D | +++ |
| 10 | A | +++ | A | +++ |
| 13 | C | +++ | C | + |
| 14 | C | +++ | B | ++ |
| 15 | A | +++ | D | +++ |
| 18 | A | +++ | A | ++ |
| 19 | A | +++ | A | +++ |
| 20 | A | +++ | A | +++ |
| 21 | B | +++ | D | +++ |
| 22 | A | +++ | D | ++ |
| 23 | A | ++ | A | ++ |

EC₅₀ (nM) range: A: EC₅₀ < 500; B: 500 < EC₅₀ < 1000; C: 1000 < EC₅₀ < 1500; D: 1500 < EC₅₀
% Eₘₐₓ range (to ALLO): +++: Eₘₐₓ > 90%; ++: 90% > Eₘₐₓ > 50%; +: Eₘₐₓ < 50%

What is claimed is:

1. A neuroactive steroid (NAS) according to formula (1): 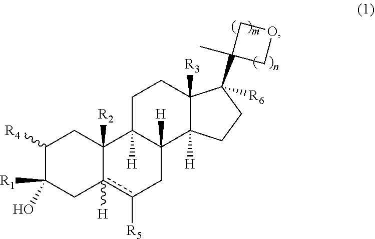
one or more isomers thereof, a deuterium labeled variant thereof, or a combination thereof,
wherein:
R₁ is H, D, substituted or unsubstituted C1-C10 alkyl, C1-C5 deuterated alkyl; substituted or unsubstituted C2-C10 alkenyl, substituted or unsubstituted C2-C10 alkynyl, substituted or unsubstituted C3-C10 cycloalkyl, substituted or unsubstituted C3-C10 cycloalkenyl, substituted or unsubstituted C3-C10 heterocycloalkyl, substituted or unsubstituted C3-C10 heterocycloalkenyl, substituted or unsubstituted aryl, or substituted or unsubstituted heteroaryl;
R₂, R₄ and R₅ each is independently H, halogen, —CN, substituted or unsubstituted C1-C10 alkyl, substituted or unsubstituted C2-C10 alkenyl, substituted or unsubstituted C2-C10 alkynyl, substituted or unsubstituted C3-C10 cycloalkyl, substituted or unsubstituted C3-C10 cycloalkenyl, substituted or unsubstituted C3-C10 heterocycloalkyl, substituted or unsubstituted C3-C10 heterocycloalkenyl, substituted or unsubstituted aryl, substituted or unsubstituted heteroaryl, or a combination thereof;
R₃ is H, D, halogen, —CN, substituted or unsubstituted C1-C10 alkyl, —CD₃, substituted or unsubstituted C2-C10 alkenyl, substituted or unsubstituted C2-C10 alkynyl, substituted or unsubstituted C3-C10 cycloalkyl, substituted or unsubstituted C3-C10 cycloalkenyl, substituted or unsubstituted C3-C10 heterocycloalkyl, substituted or unsubstituted C3-C10 heterocycloalkenyl, substituted or unsubstituted aryl, substituted or unsubstituted heteroaryl, or a combination thereof;
R₆ is H or D; and
m and n each is independently 0, 1, 2 or 3, with the proviso that at least one of m and n is not 0.

2. The neuroactive steroid of claim 1, wherein at least one of said R₁, R₂, R₃, R₄ and R₅ is a C1-C10 haloalkyl, wherein said halogen is one or more Cl, F, Br or I.

3. The neuroactive steroid of claim 1 or 2, wherein at least one of said R₁, R₂, R₃, R₄ and R₅ is a substituted or unsubstituted C3-C10 cycloalkyl, substituted or unsubstituted C3-C10 cycloalkenyl, substituted or unsubstituted C3-C10 heterocycloalkyl, substituted or unsubstituted C3-C10 heterocycloalkenyl, substituted or unsubstituted aryl, or substituted or unsubstituted heteroaryl.

4. The neuroactive steroid of any one of claims 1-3, wherein R₁ is H or C1-C5 alkyl.

5. The neuroactive steroid of any one of claims 1-4, wherein R₁ is H.

6. The neuroactive steroid of any one of claims 1-4, wherein R₁ is methyl or ethyl.

7. The neuroactive steroid of any one of claims 1-4, wherein R₁ is methyl.

8. The neuroactive steroid of any one of claims 1-7, wherein R₂ is H or C1-C5 alkyl.

9. The neuroactive steroid of any one of claims 1-7, wherein R₂ is H.

10. The neuroactive steroid of any one of claims 1-7, wherein R₂ is methyl or ethyl.

11. The neuroactive steroid of any one of claims 1-7, wherein R₂ is methyl.

12. The neuroactive steroid of any one of claims 1-11, wherein R₃ is H, -D, —CH₃, —CD₃, —CN, substituted or unsubstituted cyclopropyl, substituted or unsubstituted C1-C10 haloalkyl, substituted or unsubstituted C3-C10 heterocycloalkyl, substituted or unsubstituted C3-C10 heterocycloalkenyl, substituted or unsubstituted aryl, substituted or unsubstituted heteroaryl, —X, 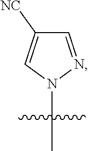
wherein X is selected from the group consisting of Cl, F, Br and I.

13. The neuroactive steroid of any one of claims 1-12, wherein R₃ is selected from the group consisting of H, -D, F, —CH₃, —CD₃, —CH₂-cyclopropyl, —CH₂OH, —COOH, —CH₂CN, —CH₂F, —CHF₂, —CH₂CF₃, —C≡CH, -cyclopropyl, —CN,

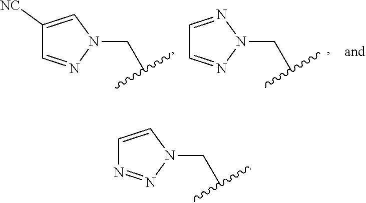 [SMILES: *Cn1cc(C#N)cn1 ; *Cn1nccn1 ; *Cn1ccnn1]

14. The neuroactive steroid of any one of claims 1-13, wherein R₃ is H, D, F, —CH₃, —CD₃, and —CN.

15. The neuroactive steroid of any one of claims 1-14, wherein R₄ is H or substituted or unsubstituted C1-C5 alkyl.

16. The neuroactive steroid of any one of claims 1-14, wherein R₄ is H.

17. The neuroactive steroid of any one of claims 1-16, wherein R₅ is H or substituted or unsubstituted C1-C5 alkyl.

18. The neuroactive steroid of any one of claims 1-17, wherein R₅ is H.

19. The neuroactive steroid of any one of claims 1-18, wherein said neuroactive steroid is:

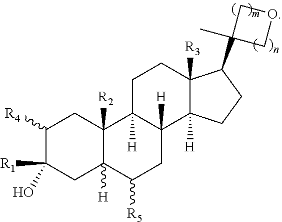

20. The neuroactive steroid of any one of claims 1-18, wherein said neuroactive steroid is:

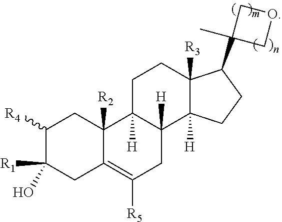

21. The neuroactive steroid of claim 1, wherein said neuroactive steroid is: 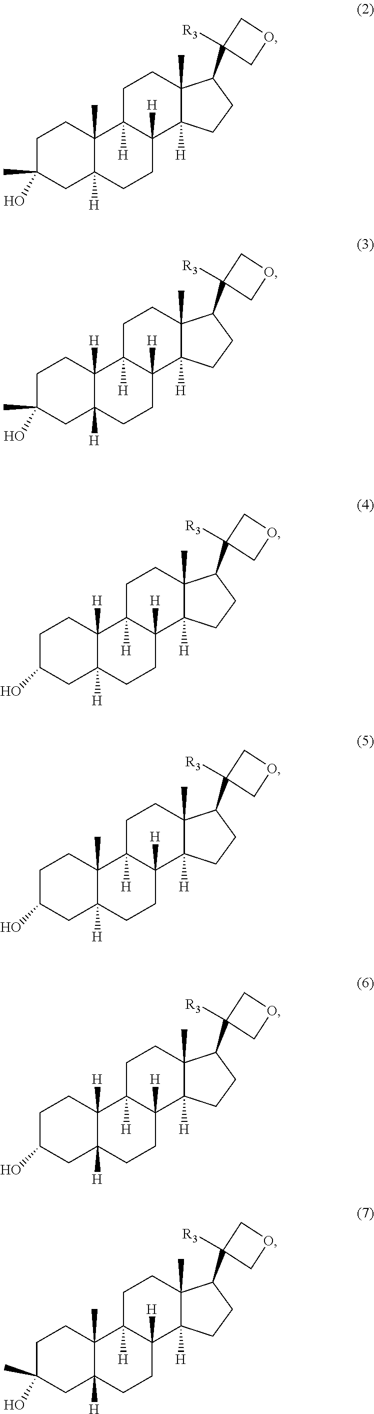
one or more isomers thereof, or a combination thereof.

22. The neuroactive steroid of claim 21, wherein said R₃ is H, -D, —CH₃, —CD₃, —CN, substituted or unsubstituted cyclopropanyl, substituted or unsubstituted C1-C10 haloalkyl, substituted or unsubstituted C3-C10 heterocycloalkyl, substituted or unsubstituted C3-C10 heterocycloalkenyl, substituted or unsubstituted aryl or substituted or unsubstituted heteroaryl, —X, or 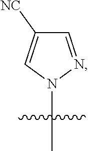 [SMILES: *n1cc(C#N)cn1]
wherein said X is Cl, F, Br or I.

23. The neuroactive steroid of claim 22, wherein said C1-C10 haloalkyl is —CXH₂, —CX₂H, —CX₃, —CH₂CXH₂, —CH₂CX₂H, or —CH₂CX₃, and wherein X is Cl, F, Br, I.

24. The neuroactive steroid of claim 23, wherein said C1-C10 haloalkyl is —CFH₂, —CF₂H, —CF₃, —CH₂CFH₂, —CH₂CF₂H or —CH₂CF₃.

25. The neuroactive steroid of claim 21, wherein said R₃ is:

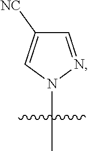 [SMILES: *n1cc(C#N)cn1]

26. The neuroactive steroid of claim 21, wherein said R₃ is H, D, F, —CH₃, —CD₃, and —CN.

27. A pharmaceutical composition comprising a neuroactive steroid (NAS) any one of claims 1-26; and a pharmaceutically acceptable excipient.

28. A method for treating a disease in a subject in need thereof, said method comprising administering said subject an effective dosage of the pharmaceutical composition of claim 27.

29. The method of claim 28, wherein said pharmaceutical composition is administered to said subject via intramuscular (IM) injection, subcutaneous (SC) injection, intravenous (IV) injection, oral administration, topical application, implant application or a combination thereof.

30. The method of any one of claim 28 or 29, wherein said disease comprises sleep disorders, insomnia, mood disorders, depression, dysthymic disorder, mild depression, bipolar disorder, anxiety disorders, generalized anxiety disorder (GAD), social anxiety disorder, stress, post-traumatic stress disorder (PTSD), compulsive disorders, obsessive compulsive disorder (OCD), schizophrenia spectrum disorders, schizophrenia, schizoaffective disorder, convulsive disorders, epilepsy, status epilepticus (SE), seizures, disorders of memory and/or cognition, attention disorders, attention deficit hyperactivity disorder (ADHD), dementia, Alzheimer's type dementia, Lewis body type dementia, vascular type dementia, movement disorders, Huntington's disease, Parkinson's disease, personality disorders, anti-social personality disorder, obsessive compulsive personality disorder, autism spectrum disorders (ASD), autism, monogenetic causes of autism, synaptophathy's, Rett syndrome, Fragile X syndrome, Angelman syndrome, neuropathic pain, injury related pain syndromes, acute pain, chronic pain, traumatic brain injury (TBI), vascular diseases, stroke, ischemia, vascular malformations, substance abuse disorders and/or withdrawal syndromes, addition to opiates, addition to cocaine, addition to alcohol, tinnitus, or a combination thereof.

31. The method of any one of claims 28-30, wherein said disease comprises CDD, MDD, PPD, essential tremor, PTSD, SE, ESE, Fragile X syndrome, Parkinson's Disease, or treatment resistant depression.

32. Use of the neuroactive steroid of any one of claims 1-26 for manufacturing a medicament for treating a disease, wherein said disease comprises sleep disorders, insomnia, mood disorders, depression, dysthymic disorder, mild depression, bipolar disorder, anxiety disorders, generalized anxiety disorder (GAD), social anxiety disorder, stress, post-traumatic stress disorder (PTSD), compulsive disorders, obsessive compulsive disorder (OCD), schizophrenia spectrum disorders, schizophrenia, schizoaffective disorder, convulsive disorders, epilepsy, status epilepticus (SE), seizures, disorders of memory and/or cognition, attention disorders, attention deficit hyperactivity disorder (ADHD), dementia, Alzheimer's type dementia, Lewis body type dementia, vascular type dementia, movement disorders, Huntington's disease, Parkinson's disease, personality disorders, anti-social personality disorder, obsessive compulsive personality disorder, autism spectrum disorders (ASD), autism, monogenetic causes of autism, synaptophathy's, Rett syndrome, Fragile X syndrome, Angelman syndrome, neuropathic pain, injury related pain syndromes, acute pain, chronic pain, traumatic brain injury (TBI), vascular diseases, stroke, ischemia, vascular malformations, substance abuse disorders and/or withdrawal syndromes, addition to opiates, addition to cocaine, addition to alcohol, tinnitus, or a combination thereof.

33. The use of claim 32, wherein said disease comprises CDD, MDD, PPD, essential tremor, PTSD, SE, ESE, Fragile X syndrome, Parkinson's Disease, or treatment resistant depression.
